(19) United States
(12) Patent Application Publication
(10) Pub. No.: US 2025/0154147 A1
(43) Pub. Date: May 15, 2025

(54) NOVEL AMINE-SUBSTITUTED PHTHALAZINES AND DERIVATIVES AS SOS1 INHIBITORS

(71) Applicant: Jazz Pharmaceuticals Ireland Limited, Dublin (IE)

(72) Inventors: Clifford David JONES, Macclesfield (GB); Gayle DOUGLAS, Macclesfield (GB); Camille GIGNOUX, Macclesfield (GB); Robin Charles HUMPHREYS, Dillon, CO (US)

(21) Appl. No.: 18/727,926

(22) PCT Filed: Jan. 13, 2023
(86) PCT No.: PCT/EP2023/050753

§ 371 Date: Jul. 10, 2024

(30) Foreign Application Priority Data
Jan. 14, 2022 (GB) 2200463.4

(51) Int. Cl. C07D 471/04; A61K 31/502

(52) U.S. Cl. CPC C07D 471/04; A61K 31/502

(57) ABSTRACT

The present disclosure provides compounds of formula (Ia) that are inhibitors of SOS1, pharmaceutical compositions thereof, and methods of treating oncological diseases using the compounds and compositions disclosed herein.

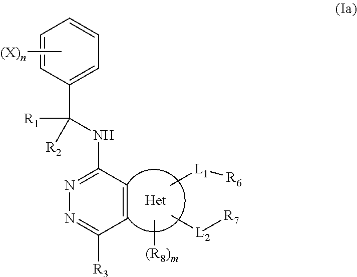

### CROSS-REFERENCE TO RELATED APPLICATIONS

This application claims the benefit of and priority to Application No. GB2200463.4, filed Jan. 14, 2022, which is incorporated herein by reference in its entirety.

### BACKGROUND

RAS proteins are a family of GTPases including KRAS (Kirsten rat sarcoma virus), NRAS (Neuroblastoma RAS viral oncogene homolog), HRAS (Harvey Rat sarcoma virus) and their respective mutants, that in cells exist in either GTP-bound or GDP-bound states. RAS proteins are critical signal transduction regulators that regulate cell proliferation, differentiation, migration and survival in different cell types. They play an important role in human cancer, with RAS oncogenic mutations identified in 20-30% of all human tumours, and for example are recognised as tumorigenic drivers in lung, colorectal and pancreatic cancers (Malumbres et al., 2001 Nature Reviews Cancer, 322-331; Pylayeva-Gupta et al., 2011 Nature Reviews Cancer, 761-774).

Acting as molecular switches, RAS proteins cycle between an active (GTP-bound) and an inactive (GDP-bound) state. Both GTPase activating proteins (GAPs) and guanine nucleotide exchange factors (GEFs) tightly regulate the activity status of RAS proteins. GAPs, such as NF1, deactivate RAS-GTP by stimulating the intrinsic GTPase catalytic activity of RAS proteins, leading to the hydrolysis and release of the gamma-phosphate of the bound GTP, resulting in inactive GDP-bound RAS protein. Binding of GEFs, such as SOS (Son of Sevenless) activate RAS proteins by stimulating the release of GDP thereby enabling the subsequent binding of the more abundant GTP, resulting in active GTP-bound RAS protein. Activated RAS proteins can signal through several downstream effector pathways, such as the RAF-MEK-ERK or Pi3K-Akt pathways. Cancer-associated mutations in RAS proteins suppress their ability to hydrolyse bound-GTP, even in presence of GAPs, leading to increased levels of active GTP-bound mutated RAS proteins (McCormick et al., 2015 Expert Opin. Ther. Targets, 19(4), 451-454). This in turn results in persistent activation of effector pathways downstream of RAS proteins.

The most widely studied RAS-GEF is the protein SOS, for which 2 human isoforms are known (SOS1 and SOS2). SOS1 and SOS2 both share 70% sequence similarity, with around 80% in the catalytic domain, but are both involved in different protein-protein interaction with RAS. Most studies suggest a dominant functional role of SOS1 over SOS2 in various physiological and pathological contexts (Baltanas et al., 2020 BBA Reviews on Cancer). SOS1 is a large multidomain protein of 1333 amino acids, consisting of 2 tandem N-terminal histone domains (HD) followed by a Dbl homology domain (DH), a Pleckstrin domain (PH), a helical linker (HL), a RAS exchange domain (REM), a CDC25 domain and a C-terminal proline rich domain (PR). The REM and CDC25 domains form the catalytic site involved in the nucleotide exchange activity on GDP-bound RAS (Kim et al., 1998 Oncogene 2597-2607). SOS1 also has an allosteric site, located between the CDC25 and the REM domains, that binds GTP-bound RAS proteins resulting in a further increase in the catalytic GEF function of SOS1 (Freedman et al., 2006 Proc. Natl. Acad. Sci. USA 16692-16697).

SOS1 has been shown to play an essential role in mutant KRAS activation and oncogenic signaling (Jeng et al., 2012 Nat. Commun., 3:1168). Oncogenic mutant KRAS activates wild-type (WT) RAS proteins through allosteric stimulation of SOS1 and this SOS1-mediated cross-activation of WT-RAS proteins contributes to cancer cell proliferation. Published data also indicates that SOS1 is involved in the activation of RAS protein signaling in cancer through mechanisms other than RAS mutations. The adaptor protein Grb2 associates with SOS1 via the binding of the Grb2 SH3 domains to the PR region of SOS1, and the complex becomes recruited to phosphorylated receptor tyrosine kinases (RTKs), for example EGFR or ALK through binding of the SH2 domains of Grb2 (Pierre et al., 2011 Biochem. Pharmacol., 82(9) 1049-1056). The SOS1-Grb2 complex also interacts with the oncoprotein Bcr-Abl, which is found in chronic myelogenous leukaemia (Kardinal et a., 2001 Blood, 98(6) 1773-1781). Other activated cell surface receptors like T-cell receptor, B-cell receptor and monocyte colony-stimulating factor receptor can recruit SOS1 to the plasma membrane, resulting in RAS-family protein activation (Salojin et al., 2000 J. Biol. Chem., 275(8) 5966-5975). SOS1 mutations in cancer are rare but can be present in many sporadic tumours including lung adenocarcinoma, urothelial bladder cancer and cutaneous melanoma. Furthermore, SOSi mutations are also found in RASopathies such as Noonan syndrome and hereditary gingival fibromatosis (Baltanas et al., 2020 BBA Reviews on Cancer). In addition, SOS1 acts as GEF for the GTPase RAC, a member of the Rho subfamily of small GTPases, which is involved in angiogenesis and metastasis (Bid et al., 2013 Mol. Cancer Ther., 12(10) 1925-1934), although this is through SOS1 protein domains (PH-DH domains) distinct from those involved in RAS protein activation (REM-CDC25 domains).

The homolog SOS2 also acts as a GEF for RAS and RAC proteins (Pierre et al., 2011 Biochem. Pharmacol., 82(9) 1049-1056). Studies have showed that SOS2 is completely dispensable for mouse development, since SOS2 knockout mice survive to adulthood and were found to be viable and fertile, whereas SOS1 germline-null animals die during mid-gestation (Esteban et al., 2000 Mol. Cell. Biol., 20(17) 6410-6413; Qian et al., 2000 EMBO J., 19(4) 642-654). The systemic conditional knockout of SOS1 in adult mice demonstrated that SOS1 loss in adults is viable, whereas the equivalent SOS1/2 double knockout adult mice die precociously. This suggests functional redundancy in adults between SOS1 and SOS2 for lymphopoiesis, homeostasis and survival (Baltanas et al., 2013 Mol. Cell. Biol., 2013 33(22) 4562-4578). Selective inhibition of SOS1 functions over SOS2 may therefore represent a safe and viable approach for targeting RAS-driven tumors and pathologies.

Due to its role in the RAS protein mediated signaling pathways, SOS1 is an attractive target for cancer therapy. Recently, small molecules which selectively bind SOS1 and prevent its protein-protein interaction with RAS proteins have been reported. These compounds attenuate or eliminate the downstream effector events of RAS-mediated pathways e.g., ERK phosphorylation (Hillig et al., 2019 Proc. Natl. Acad. Sci. USA, 116(7) 2551-2560; Hofmann et al., 2020 Cancer Discovery, 142-157). In addition, several patent applications related to SOS1 inhibitors are published: WO2004003152, WO2016077793, WO2018115380, WO2018172250, WO2019122129, WO2019201848, WO2020180768, WO2020180770, WO2021092115, WO2021105960, WO2021124429, WO2021130731, WO2021173524.

### SUMMARY

In one aspect, the present disclosure provides a compound of Formula (I):

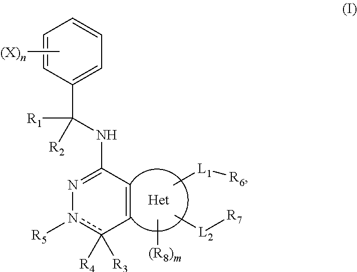

or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen;
R₃ and R₄ are independently absent, hydrogen, alkyl, —(C═O)—OR_{A}, —(C═O)—N(R_{A})₂, cycloalkyl, heterocyclyl, aryl or heteroaryl, or R₃ and R₄ together form a carbonyl (i.e., an oxo group), wherein R_{A} is selected from the group consisting of hydrogen and alkyl;
R₅ is absent, hydrogen, alkyl, or cycloalkyl;

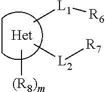

a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
X is independently selected from the group consisting of C₁₋₅alkyl, F, CF₃, CHF₂, CH₂F, and —NH₂;
m is 1 or 2;
n is an integer from 1-5; and
[symbol] is a single or double bond,
provided that

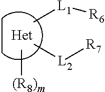

is not

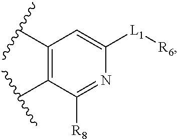

and
when [symbol] is a double bond, R₄ and R₅ are absent.

In some embodiments, the compound of Formula (I) is not one or more of:

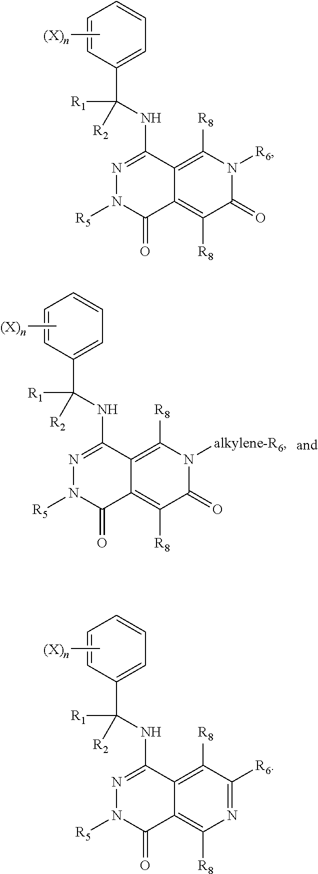

In another aspect, the present disclosure provides a compound of Formula (Ia):

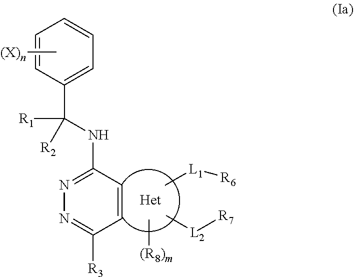

or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen; and
R₃ is absent, hydrogen, alkyl, —(C═O)—OR_{A}, —(C═O)—N(R_{A})₂, cycloalkyl, heterocyclyl, aryl or heteroaryl, wherein R_{A} is selected from the group consisting of hydrogen and alkyl

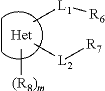

is a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
X is independently selected from the group consisting of C₁₋₅alkyl, F, CF₃, CHF₂, CH₂F, and —NH₂;
m is 1 or 2; and
n is an integer from 1-5;
provided that

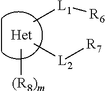

is not

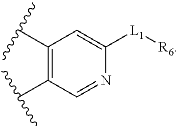

In some embodiments, the compound of Formula (I) or Formula (Ia) is not:

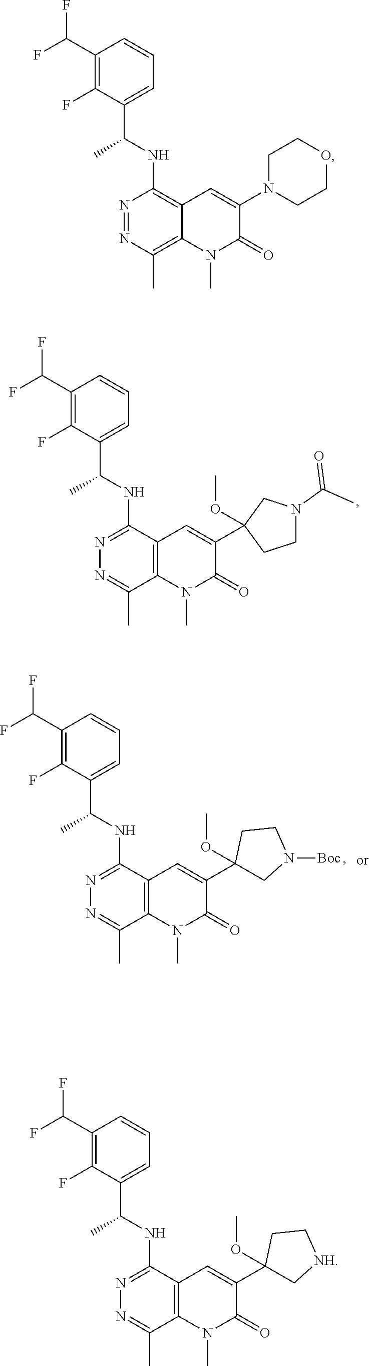 [SMILES: Cc1nnc(N[C@H](C)c2cccc(C(F)F)c2F)c2cc(N3CCOCC3)c(=O)n(C)c12 ; COC1(c2cc3c(N[C@H](C)c4cccc(C(F)F)c4F)nnc(C)c3n(C)c2=O)CCN(C(C)=O)C1 ; COC1(c2cc3c(N[C@H](C)c4cccc(C(F)F)c4F)nnc(C)c3n(C)c2=O)CCN(C(=O)OC(C)(C)C)C1 ; "," ; COC1(c2cc3c(N[C@H](C)c4cccc(C(F)F)c4F)nnc(C)c3n(C)c2=O)CCNC1]

In some embodiments,

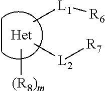

is selected from the group consisting of:

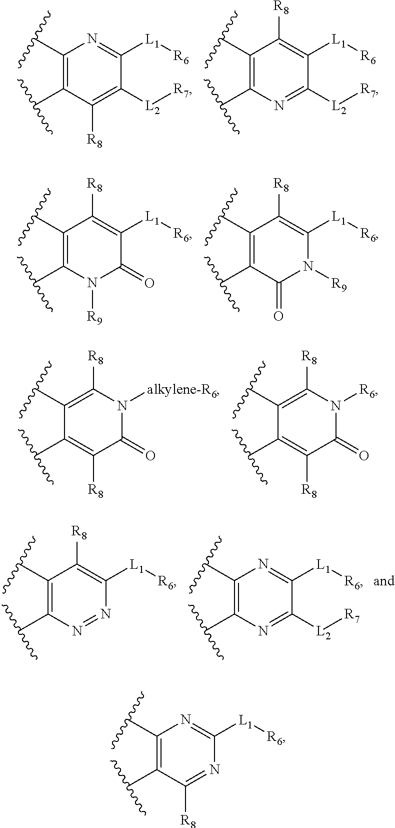

wherein R₈ is each independently H, halogen, alkyl, alkoxy, or —CH₂—O-alkyl and R₉ is H, alkyl, cycloalkyl, heterocyclyl, alkylene-cycloalkyl, or alkylene-heterocyclyl.

In some embodiments, the linking group is selected from the group consisting of —O—, alkylene, alkylene-O—, alkylene-N(R_{B})—, —O-alkylene, —N(R_{B})-alkylene, —O—, and —N(R_{B})—, wherein R_{B} is hydrogen, alkyl, and alkylenecycloalkyl.

In some embodiments, L₁ and L₂ are each independently a linking group selected from the group consisting of alkylene, —O-alkylene, —N(R_{B})-alkylene, —O—, and —N(R_{B})—, wherein R_{B} is hydrogen, alkyl, or alkylenecycloalkyl. In some embodiments, L₁ and L₂ are each independently absent or a linking group selected from the group consisting of alkylene, —O-alkylene, —N(R_{B})-alkylene, —O—, and —N(R_{B})—, wherein R_{B} is hydrogen, alkyl, or alkylenecycloalkyl. In some embodiments, L₁ is a linking group selected from the group consisting of alkylene, —O-alkylene, —N(R_{B})-alkylene, and —O—, and —N(R_{B})—, wherein R_{B} is hydrogen, alkyl, or alkylenecycloalkyl and L₂ is absent or —O—. In some embodiments, L₁ is alkylene or —O— and L₂ is absent or —O—. In some embodiments, L₁ and L₂ are each independently is —O—. In some embodiments, L₁ is a linking group selected from the group consisting of alkylene, —O-alkylene, —N(R_{B})-alkylene, —O—, and —N(R_{B})—, wherein R_{B} is hydrogen, alkyl, or alkylenecycloalkyl and L₂ is absent. In some embodiments, L₁ is —O— and L₂ is absent. In some embodiments, L₂ is a linking group selected from the group consisting of alkylene, —O-alkylene, —N(R_{B})-alkylene, —O—, and —N(R_{B})—, wherein R_{B} is hydrogen, alkyl, or alkylenecycloalkyl and L₁ is absent. In some embodiments, L₂ is —O— and Li is absent.

In some embodiments, each X is independently —CF₂CH₃, —CF₂CH₂OH, —CF₂C(CH₃)₂OH, —CHF₂, —CF₃, F, or —NH₂.

In some embodiments,

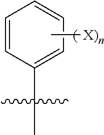

is selected from the group consisting of:

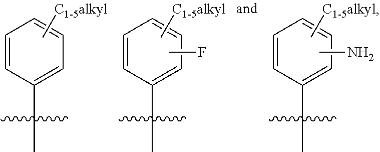

wherein the C₁₋₅alkyl is a C₁₋₅haloalkyl. In some embodiments, the C₁₋₅alkyl is a C₁₋₅fluoroalkyl. In some embodiments, the C₁₋₅alkyl is selected from the group consisting of —CF₂CF₃, —CF₂CH₃, —CF₂CH₂OH, —CF₂C(CH₃)₂OH, —CHF₂, —CF₃, and —CH₂F.

In some embodiments,

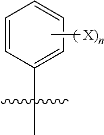

is selected from the group consisting of:

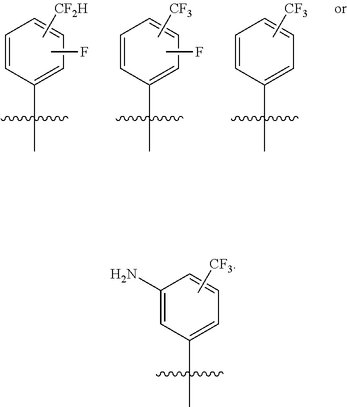

In some embodiments, n is 1 or 2. In some embodiments, n is 1. In some embodiments, n is 2.

In some embodiments, [symbol] is a double bond. In some embodiments, [symbol] is a single bond.

In some embodiments, R₁ is alkyl and R₂ is H. In some embodiments, R₁ is C₁₋₅alkyl and R₂ is H. In some embodiments, R₁ is methyl and R₂ is H. In some embodiments, R₂ is alkyl and R₁ is H. In some embodiments, R₂ is C₁₋₅alkyl and R₁ is H. In some embodiments, R₂ is methyl and R₁ is H.

In some embodiments, R₃ and R₄ are each independently selected from the group consisting of H, methyl, ethyl, isopropyl, n-propyl, —CH₂OH, —CH₂OCH₃, —CH₂N(CH₃)₂, —CH(OH)(CH₃)₂ and —CH₂(OH)CH₃. In some embodiments, R₃ is selected from the group consisting of H, methyl, ethyl, isopropyl, n-propyl, —CH₂OH, —CH₂OCH₃, —CH₂N(CH₃)₂, —CH(OH)(CH₃)₂ and —CH₂(OH)CH₃ and R₄ is H. In some embodiments, R₃ is selected from the group consisting of H, methyl, ethyl, isopropyl, n-propyl, —CH₂OH, —CH₂OCH₃, —CH₂N(CH₃)₂, —CH(OH)(CH₃)₂ and —CH₂(OH)CH₃ and R₄ is absent (i.e., when [symbol] is a double bond). In some embodiments, R₃ is H or C₁₋₅alkyl and R₄ is absent (i.e., when [symbol] is a double bond). In some embodiments, R₃ is H or methyl and R₄ is absent (i.e., when [symbol] is a double bond).

In some embodiments, [symbol] is a single bond and R₅ is alkyl. In some embodiments, [symbol] is a single bond and R₅ is C₁₋₅alkyl. In some embodiments, [symbol] is a single bond and R₅ is methyl. In some embodiments, when [symbol] is a single bond, R₃ and R₄ taken together form a carbonyl (i.e., an oxo group).

In some embodiments, R₆ is alkyl, cycloalkyl, or heterocyclyl. In some embodiments, R₆ is heterocyclyl. In some embodiments, the heterocyclyl is a 5- or 6-membered heterocyclyl having 1 or 2 heteroatoms selected from N, O, or S. In some embodiments, heterocyclyl is a morpholinyl, piperazinyl, piperidinyl, pyrrolidinyl, azetidinyl, tetrahydropyranyl, or tetrahydrofuranyl. In some embodiments, heterocyclyl is a piperazinyl, piperidinyl, azetidinyl, tetrahydropyranyl, or tetrahydrofuranyl. In some embodiments, the heterocyclyl is 3-tetrahydrofuranyl. In some embodiments, R₆ is cycloalkyl. In some embodiments, the cycloalkyl is a C₃₋₆cycloalkyl. In some embodiments, the cycloalkyl is cyclopentyl. In some embodiments, R₆ is alkyl. In some embodiments, R₆ is C₁₋₅alkyl. In some embodiments, the alkyl is methyl. In some embodiments, R₆ is cyclopentyl or 3-tetrahydrofuranyl.

In some embodiments, R₇ is halogen, alkyl, cycloalkyl, heterocyclyl, or heteroaryl. In some embodiments, R₇ is alkyl, cycloalkyl, heterocyclyl, or heteroaryl. In some embodiments, R₇ is heterocyclyl. In some embodiments, the heterocyclyl is 3-tetrahydrofuranyl. In some embodiments, R₇ is cycloalkyl. In some embodiments, the cycloalkyl is a C₃₋₆cycloalkyl. In some embodiments, the cycloalkyl is cyclopentyl. In some embodiments, R₇ is alkyl. In some embodiments, the alkyl is a C₁₋₅alkyl. In some embodiments, the alkyl is methyl. In some embodiments, R₇ is cyclopentyl or 3-tetrahydrofuranyl. In some embodiments, R₇ is H, halogen, C₁₋₅alkyl, C₃₋₆cycloalkyl, C₄₋₆heterocyclyl, or 5-6-membered heteroaryl. In some embodiments the C₄₋₆heterocyclyl is morpholinyl, piperazinyl, piperidinyl, pyrrolidinyl, or azetidinyl. In some embodiments, the 5- or 6-membered heteroaryl is pyrazolyl, pyridinyl, pyrimidinyl, imidazolyl, oxazolyl, or thiazolyl.

In some embodiments, R₆ is alkyl, cycloalkyl, heterocyclyl, aryl or heteroaryl and R₇ is H, halogen, or alkyl. In some embodiments, R₆ is alkyl, cycloalkyl, or heterocyclyl, and R₇ is H, halogen, or alkyl. In some embodiments, R₆ is H or alkyl and R₇ is alkyl, cycloalkyl, heterocyclyl, aryl or heteroaryl.

In some embodiments, R₈ is H, halogen, C₁₋₅alkyl, C₁₋₅alkoxy, or —CH₂—O—C₁₋₅alkyl. In some embodiments, the C₁₋₅alkyl is methyl. In some embodiments, the halogen is F or Cl. In some embodiments, the C₁₋₅alkoxy is methoxy.

In some embodiments, R₉ is H or methyl. In some embodiments, R₉ is H. In some embodiments, R₉ is methyl.

In some embodiments, the compound of the present disclosure is selected from the group consisting of:

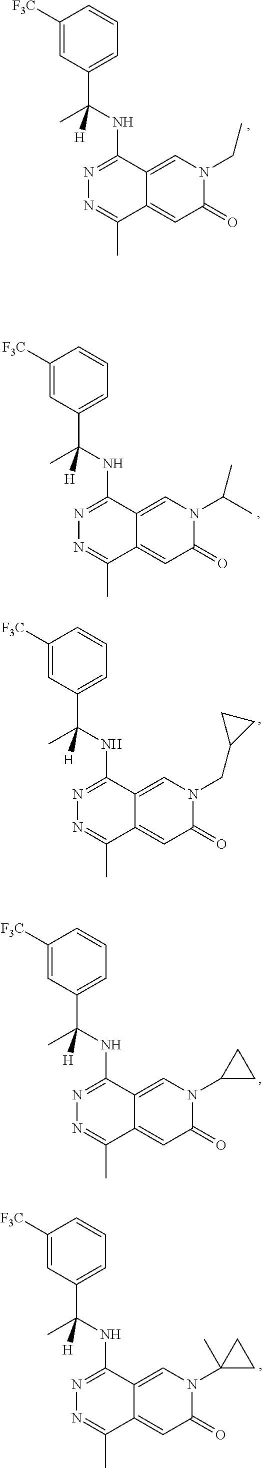

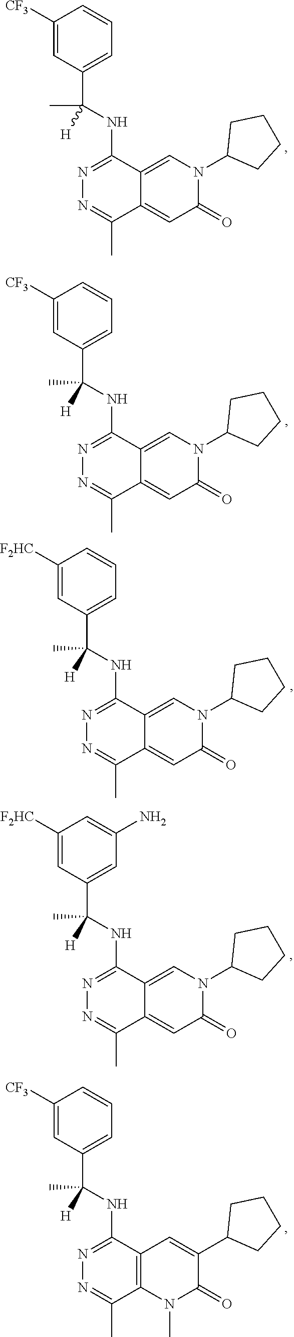

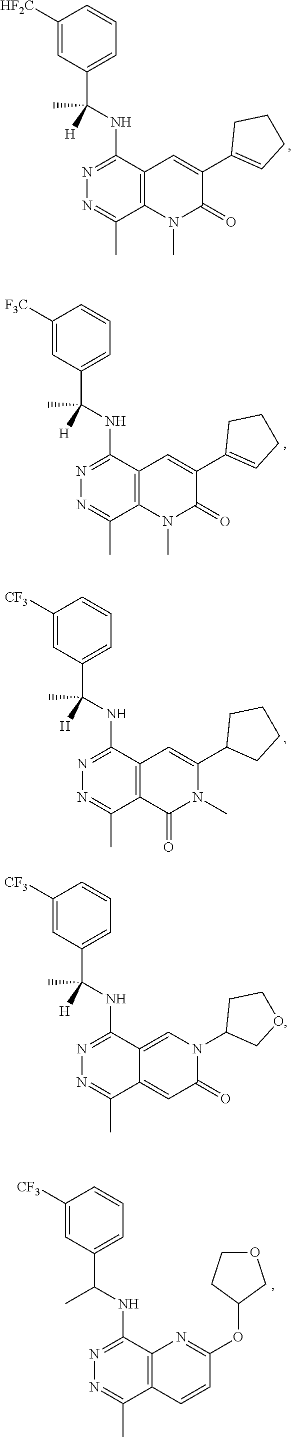

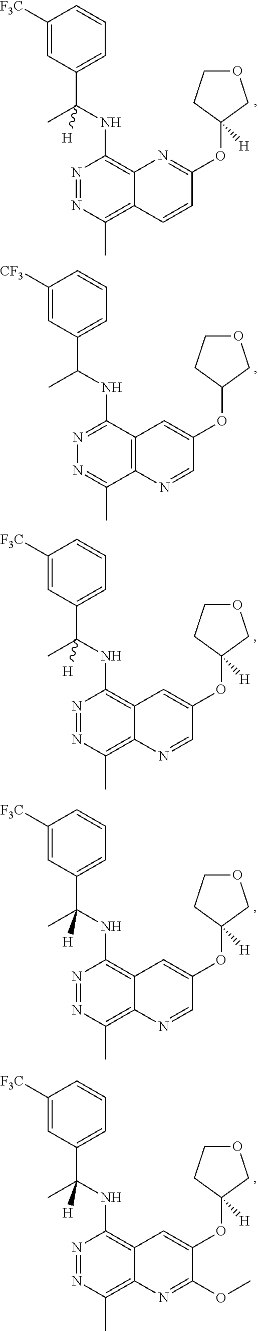

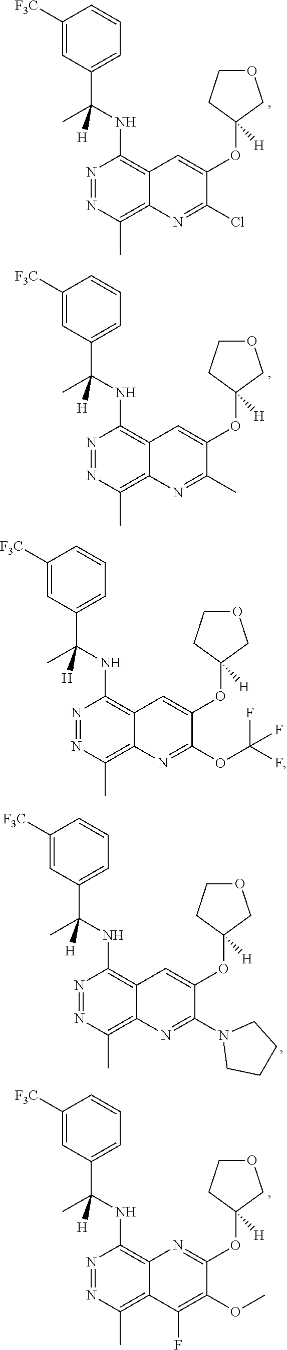

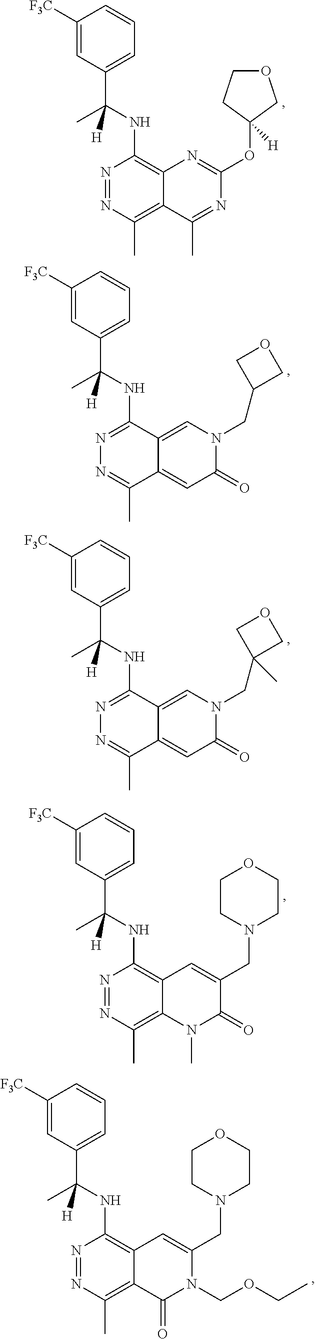

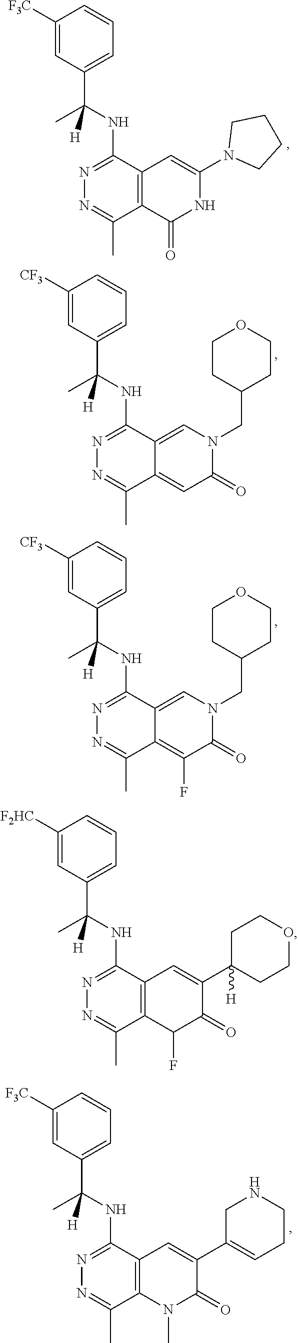

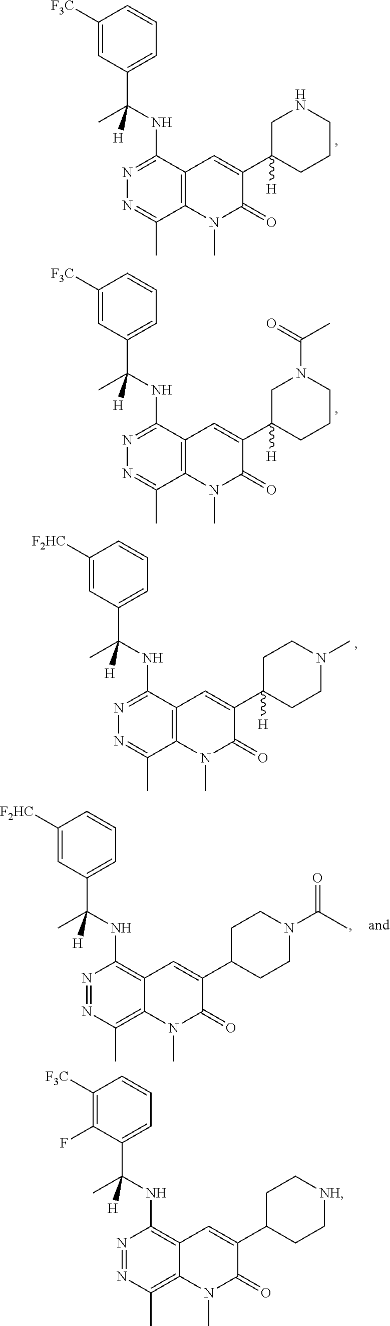

In some embodiments, the present disclosure provides a pharmaceutical composition comprising a compound disclosed herein or pharmaceutically acceptable salt thereof, and a pharmaceutically acceptable excipient.

In some embodiments, the present disclosure provides a method of treating and/or preventing cancer comprising administering to a subject a therapeutically effective amount of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Je), or Formula (If)), a pharmaceutically acceptable salt thereof, or a pharmaceutical composition thereof.

In some embodiments, the present disclosure provides a method of treating and/or preventing a disease by inhibiting the interaction of SOS1 and a RAS-family protein or RAC1, the method comprising administering to a subject a therapeutically effective amount of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)), a pharmaceutically acceptable salt thereof, or a pharmaceutical composition thereof.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity; for example, “a SOS1 inhibitor” refers to one or more SOS1 inhibitors or at least one SOS1 inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alkynyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R_{b} is an alkylene, alkenylene, or alkynylene group as defined above and R_{d} is a carbocyclyl radical as defined above. Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated or unsaturated 3- to 20-membered ring which consists of two to nineteen carbon atoms and from one to six heteroatoms selected from the group consisting of nitrogen, oxygen and sulfur, and which is attached to the rest of the molecule by a single bond. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl group. In addition, each of the foregoing substituents can also be optionally substituted with one or more of the above substituents.

As used herein, the symbol

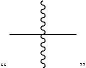

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

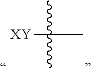

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

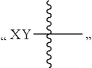

infers that when R³ is “XY”, the point of attachment bond is the same bond as the bond by which R³ is depicted as being bonded to CH₃.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient, refers to improving at least one symptom of the patient's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient in need thereof.

### COMPOUNDS OF THE DISCLOSURE

In some embodiments, the present disclosure provides a compound of Formula (I):

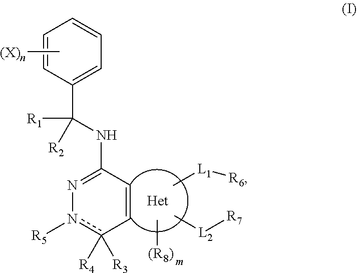

or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen;
R₃ and R₄ are independently absent, hydrogen, alkyl, —(C═O)—OR_{A}, —(C═O)—N(R_{A})₂, cycloalkyl, heterocyclyl, aryl or heteroaryl, or R₃ and R₄ together form a carbonyl, wherein R_{A} is selected from the group consisting of hydrogen and alkyl;
R₅ is absent, hydrogen, alkyl, or cycloalkyl;

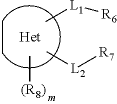

is a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
X is independently selected from the group consisting of C₁₋₅alkyl, F, CF₃, CHF₂, CH₂F, and —NH₂;
[symbol] is a single or double bond;
m is 1 or 2; and
n is an integer from 1-5;
provided that

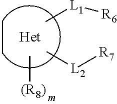

is not

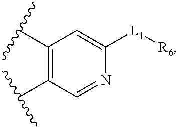

and
when [symbol] is a double bond, R₄ and R₅ are absent.

In some embodiments, the present disclosure provides a compound of Formula (I):

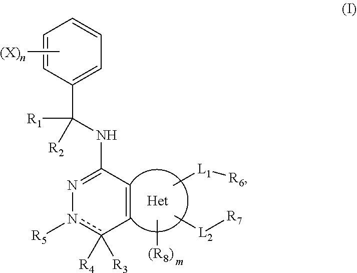

or a pharmaceutically acceptable salt thereof,
wherein:
X is independently selected from the group consisting of C₁₋₅alkyl, F, CF₃, CHF₂, CH₂F, and —NH₂;
R₁ and R₂ are independently hydrogen, alkyl, or R₁ and R₂ together with the atom to which they are attached form a cycloalkyl or heterocyclyl, wherein at least one of R₁ and R₂ is not hydrogen;
R₃ and R₄ are independently absent, hydrogen, alkyl, —(C═O)—OR_{A}, —(C═O)—N(R_{A})₂, cycloalkyl, heterocyclyl, aryl or heteroaryl, or R₃ and R₄ together form a carbonyl, wherein R_{A} is selected from the group consisting of hydrogen and alkyl;
R₅ is absent, hydrogen, alkyl, or cycloalkyl; and

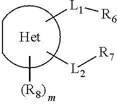

is a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
[symbol] is a single or double bond;
m is 1 or 2; and
n is an integer from 1-5,
provided that

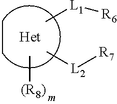

is not

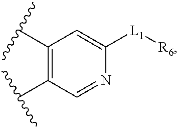

and
when [symbol] is a double bond, R₄ and R₅ are absent, and when [symbol] is a single bond, the compound is not:

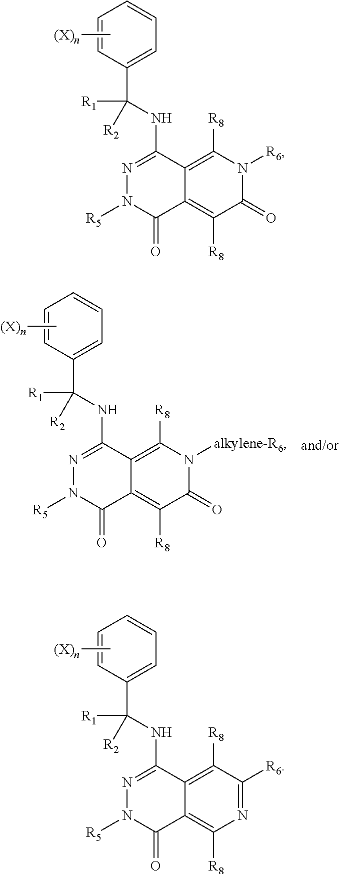

In another aspect, the present disclosure provides a compound of Formula (Ia):

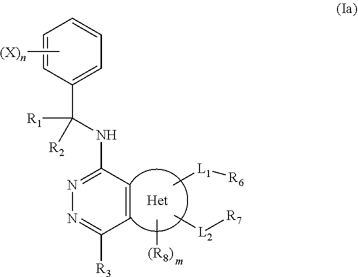

or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen; and
R₃ is absent, hydrogen, alkyl, —(C═O)—OR_{A}, —(C═O)—N(R_{A})₂, cycloalkyl, heterocyclyl, aryl or heteroaryl, wherein R_{A} is selected from the group consisting of hydrogen and alkyl

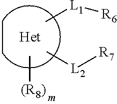

is a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
X is independently selected from the group consisting of C₁₋₅alkyl, F, CF₃, CHF₂, CH₂F, and —NH₂;
m is 1 or 2; and
n is an integer from 1-5;
provided that

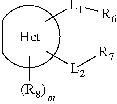

is not

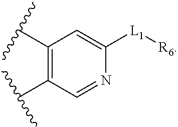

In another aspect, the present disclosure provides a compound of Formula (Ib):

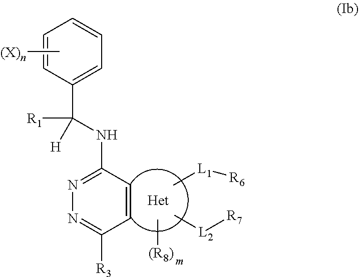

or a pharmaceutically acceptable salt thereof,
wherein:
R₁ is alkyl;
R₃ is absent, hydrogen, alkyl, —(C═O)—OR_{A}, —(C═O)—N(R_{A})₂, cycloalkyl, heterocyclyl, aryl or heteroaryl, wherein R_{A} is selected from the group consisting of hydrogen and alkyl

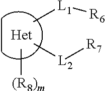

is a heteroaryl;
R₆ is H, alkyl, cycloalkyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group;
X is independently selected from the group consisting of C₁₋₅alkyl, F, CF₃, CHF₂, CH₂F, and —NH₂;
m is 1 or 2; and
n is an integer from 1-5;
provided that

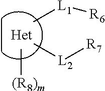

is not

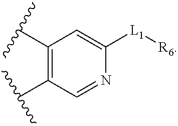

In another aspect, the present disclosure provides a compound of Formula (Ib-1):

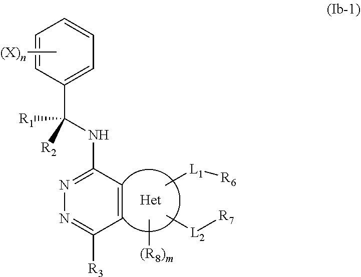

or a pharmaceutically acceptable salt thereof, wherein R₁, R₃, R₆, R₇, L₁, L₂, X, m, and n are as defined herein.

In some embodiments of Formula (I), Formula (Ia), Formula (Ib), Formula (Ib-1),

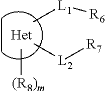

is not:

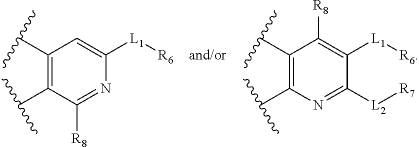

In some embodiments of Formula (I), Formula (Ia), Formula (Ib), Formula (Ib-1),

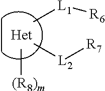

is not

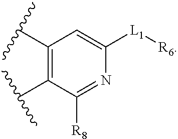

In some embodiments of Formula (I), Formula (Ia), Formula (Ib), Formula (Ib-1),

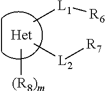

is not

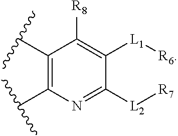

In some embodiments, the compound of the present disclosure (e.g., a compound of Formula (I), (Ia), (Ib), and (Ib-1)) is not one or more of:

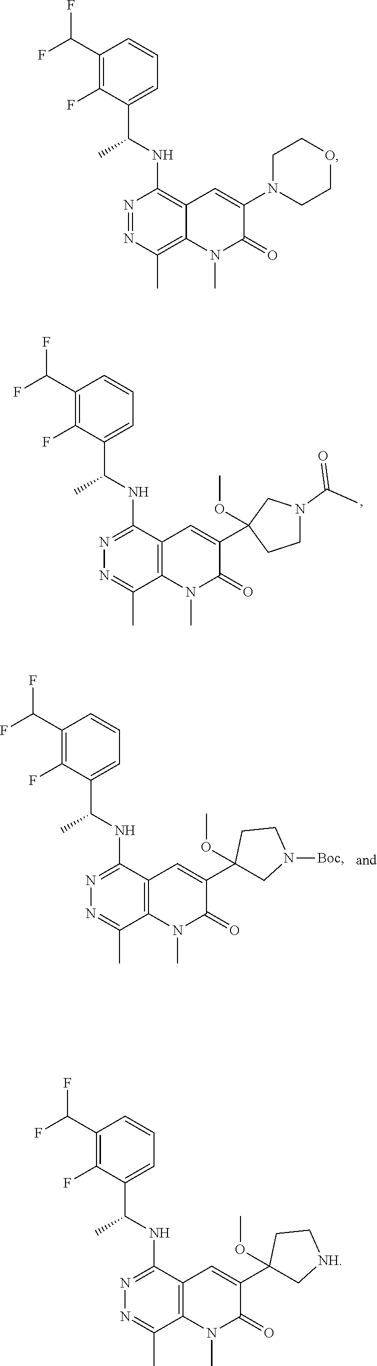 [SMILES: Cc1nnc(N[C@H](C)c2cccc(C(F)F)c2F)c2cc(N3CCOCC3)c(=O)n(C)c12 ; COC1(c2cc3c(N[C@H](C)c4cccc(C(F)F)c4F)nnc(C)c3n(C)c2=O)CCN(C(C)=O)C1 ; COC1(c2cc3c(N[C@H](C)c4cccc(C(F)F)c4F)nnc(C)c3n(C)c2=O)CCN(C(=O)OC(C)(C)C)C1 ; COC1(c2cc3c(N[C@H](C)c4cccc(C(F)F)c4F)nnc(C)c3n(C)c2=O)CCNC1]

In some embodiments, the compound of the present disclosure (e.g., a compound of Formula (I), (Ia), (Ib), and (Ib-1)) is not:

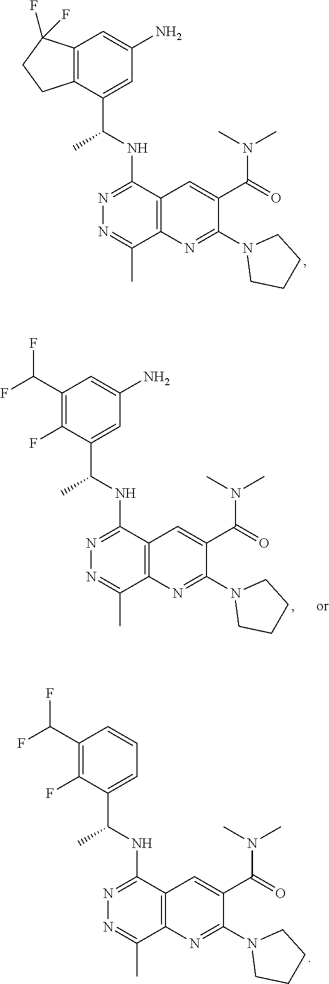 [SMILES: Cc1nnc(N[C@H](C)c2cc(N)cc3c2CCC3(F)F)c2cc(C(=O)N(C)C)c(N3CCCC3)nc12 ; Cc1nnc(N[C@H](C)c2cc(N)cc(C(F)F)c2F)c2cc(C(=O)N(C)C)c(N3CCCC3)nc12 ; Cc1nnc(N[C@H](C)c2cccc(C(F)F)c2F)c2cc(C(=O)N(C)C)c(N3CCCC3)nc12]

In some embodiments of Formula (I),

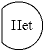

is a 5- to 14-membered heteroaryl. In some embodiments,

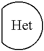

is a 5- to 14-membered heteroaryl having 1, 2, or 3 heteroatoms selected from the group consisting of N, O, and S. In some embodiments,

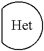

is a 5- or 6-membered heteroaryl having 1, 2, or 3 heteroatoms selected from the group consisting of N, O, and S. In some embodiments,

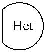

is a 6- to 14-membered heteroaryl having 1, 2, or 3 heteroatoms selected from the group consisting of N, O, and S. In some embodiments,

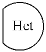

is a 5- or 6-membered heteroaryl having 1, 2, or 3 heteroatoms selected from the group consisting of N, O, and S. In some embodiments,

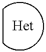

is a 6-membered heteroaryl having 1, 2, or 3 heteroatoms selected from the group consisting of N, O, and S. In some embodiments,

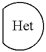

is a 6-membered heteroaryl having 1 or 2 nitrogen atoms. In some embodiments,

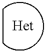

is selected from the group consisting of pyrazole, pyridine, pyrimidine, pyrazine, pyridazine, pyrimidone, pyridone, or derivative thereof. In some embodiments,

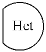

(e.g., a 6-membered heteroaryl having 1 or 2 nitrogen atoms) is selected from the group consisting of pyridine, pyrimidine, pyrazine, pyridazine, pyrimidone, pyridone, or derivative thereof. In some embodiments, the pyridone is a 2-pyridone. In some embodiments, the pyrimidine is a uracil, thymine, cytosine, or derivative thereof.

In some embodiments,

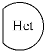

is optionally substituted with alkyl, alkoxy, halogen, oxo, —(C═O)—OR_{A}, —(C═O)—N(R_{A})₂, cycloalkyl, heterocyclyl, aryl or heteroaryl. In some embodiments,

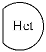

is optionally substituted with alkyl, halogen, oxo, —(C═O)—OR_{A}, —(C═O)—N(R_{A})₂, cycloalkyl, heterocyclyl, aryl or heteroaryl. In some embodiments,

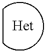

is optionally substituted with oxo, alkyl, or halogen. In some embodiments,

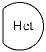

is optionally substituted with alkyl or halogen. In some embodiments,

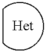

is optionally substituted with alkyl, e.g., methyl, ethyl, n-propyl, isopropyl, n-butyl, isobutyl, t-butyl, isoamyl, or neopentyl. In some embodiments,

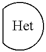

is optionally substituted with one or more halogen. In some embodiments, the halogen is F, Br, or Cl. In some embodiments,

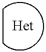

is optionally substituted with alkyl, alkoxy, halogen, —(C═O)—OR_{A}, or —(C═O)—N(R_{A})₂. In some embodiments, the alkyl is a C₁₋₅alkyl.

In some embodiments, the C₁₋₅ alkyl is methyl, ethyl, n-propyl, isopropyl, n-butyl, t-butyl, isoamyl or neopentyl. In some embodiments, the C₁₋₅alkyl is methyl. In some embodiments, the alkoxy is a C₁₋₅alkoxy, e.g., methoxy, ethoxy, propoxy, isopropoxy, butoxy, t-butoxy, and the like. In some embodiments, the cycloalkyl is a C₃₋₈ cycloalkyl. In some embodiments, the cycloalkyl is cyclopropyl. In some embodiments, the heterocyclyl is a 4- to 12-member heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the aryl is a phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, R_{A} is selected from the group consisting of hydrogen and alkyl. In some embodiments, R_{A} is hydrogen or a C₁₋₅alkyl. In some embodiments, the C₁₋₅ alkyl is methyl, ethyl, or isopropyl.

In some embodiments,

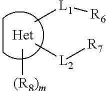

is selected from the group consisting of:

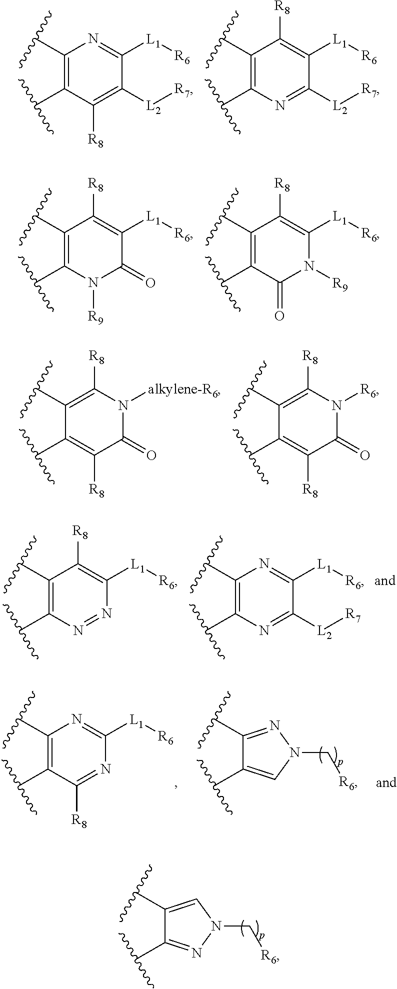

wherein L₁, L₂, R₆, R₇, and R₈ are as defined herein; R₉ is H, alkyl, —CH₂—O— alkyl, cycloalkyl, heterocyclyl, alkylene-cycloalkyl, or alkylene-heterocyclyl; and p is 0-3. In some embodiments, the alkyl is a C₁₋₅alkyl, the cycloalkyl is a C₃₋₆cycloalkyl and the heterocyclyl is a 5- or 6-membered heterocyclyl. In some embodiments, the alkylene is a C₁₋₅alkylene. In some embodiments, the alkylene is a C₁₋₃alkylene. In some embodiments, the alkylene is a methylene or ethylene. In some embodiments, the alkylene is a methylene. In some embodiments, R₉ is H, C₁₋₅alkyl, —CH₂—O—C₁₋₅alkyl, C₃₋₆cycloalkyl, or 5- to 6-membered heterocyclyl. In some embodiments, R₉ is C₁₋₅alkyl. In some embodiments, R₉ is —CH₂—O—C₁₋₅alkyl. In some embodiments, R₉ is H, methyl, ethyl, isopropyl, —CH₂—O—CH₃, —CH₂—O—CH₂CH₃, cyclopropyl, cyclopentyl, pyrrolidinyl, or piperidinyl. In some embodiments, p is 0-2. In some embodiments, p is 1 or 2. In some embodiments, p is 0. In some embodiments, p is 1. In some embodiments, p is 2.

In some embodiments,

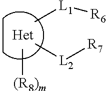

is selected from the group consisting of:

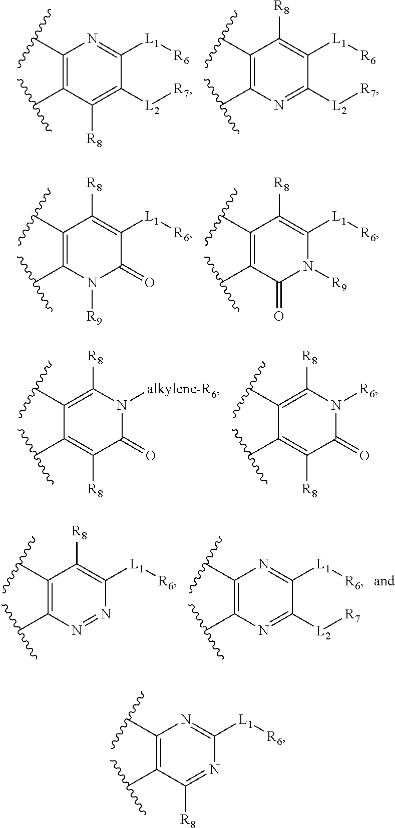

wherein L₁, L₂, R₆, R₇, and R₈ are as defined herein; and R₉ is H, alkyl, cycloalkyl, heterocyclyl, alkylene-cycloalkyl, or alkylene-heterocyclyl. In some embodiments, the alkyl is a C₁₋₅alkyl, the cycloalkyl is a C₃₋₆cycloalkyl and the heterocyclyl is a 5- or 6-membered heterocyclyl. In some embodiments, the alkylene is a C₁₋₅alkylene. In some embodiments, the alkylene is a C₁₋₃alkylene. In some embodiments, the alkylene is a methylene or ethylene. In some embodiments, the alkylene is a methylene. In some embodiments, R₉ is H, C₁₋₅alkyl, C₃₋₆cycloalkyl, or 5- to 6-membered heterocyclyl. In some embodiments, R₉ is C₁₋₅alkyl. In some embodiments, R₉ is H, methyl, ethyl, isopropyl, cyclopropyl, cyclopentyl, pyrrolidinyl, or piperidinyl

In some embodiments,

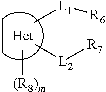

is selected from the group consisting of:

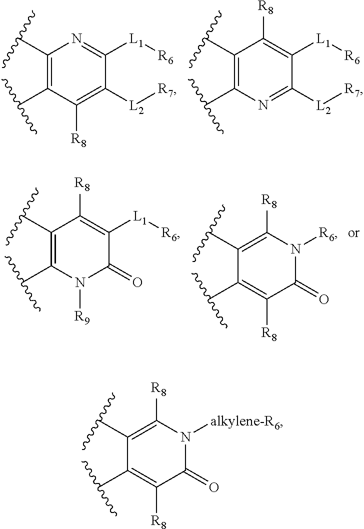

wherein L₁, L₂, R₆, R₇, R₈, and R₉ are as defined herein. In some embodiments, L₁ is absent, —CH₂—, or —O—. In some embodiments, L₂ is absent. In some embodiments, R₇ is H. In some embodiments, R₈ is H. In some embodiments, R₉ is alkyl. In some embodiments, R₉ is Me.

In some embodiments, each X is independently selected from the group consisting of halogen, alkyl, —NH₂, and alkoxy. In some embodiments, each X is independently selected from the group consisting of halogen, C₁₋₅alkyl, —NH₂, and C₁₋₅alkoxy. In some embodiments, each X is independently selected from the group consisting of halogen, C₁₋₅alkyl, and —NH₂. In some embodiments, each X is independently selected from the group consisting of C₁₋₅alkyl, F, CF₃, CHF₂, CH₂F, and —NH₂. In some embodiments, each X is independently selected from the group consisting of —CF₂CH₃, —CF₂CH₂OH, —CF₂C(CH₃)₂OH, —CH₂F, —CHF₂, —CF₃, or F, and —NH₂. In some embodiments, each X is independently selected from the group consisting of —CF₂CH₃, —CF₂CH₂OH, —CF₂C(CH₃)₂OH, —CHF₂, —CF₃, or F, and —NH₂. In some embodiments, each X is independently selected from the group consisting of —CH₂F, —CHF₂, —CF₃, or F, and —NH₂. In some embodiments, each X is independently selected from the group consisting of —CHF₂, —CF₃, or F, and —NH₂. In some embodiments, the C₁₋₅alkyl is a C₁₋₅haloalkyl. In some embodiments, the C₁₋₅alkyl is selected from the group consisting of —CF₂CF₃, —CF₂CH₃, —CF₂CH₂OH, —CF₂C(CH₃)₂OH, —CHF₂, —CF₃, and —CH₂F. In some embodiments, the C₁₋₅alkoxy is a C₁₋₅haloalkoxy. In some embodiments, the C₁₋₅haloalkoxy is selected from the group consisting of —OCF₂CF₃, —OCF₂CH₃, —OCF₂CH₂OH, —OCF₂C(CH₃)₂OH, —OCHF₂, —OCF₃, and —OCH₂F. In some embodiments, the C₁₋₅alkoxy is a C₁₋₅haloalkoxy. In some embodiments, the C₁₋₅haloalkoxy is selected from the group consisting of —OCHF₂, —OCF₃, or —OCH₂F. In some embodiments, the halogen is F, Br, or Cl. In some embodiments, the halogen is F.

In some embodiments,

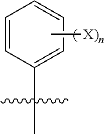

is selected from the group consisting of:

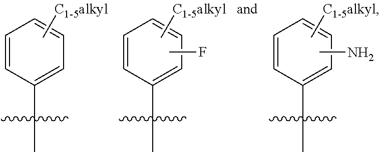

wherein the C₁₋₅alkyl is a C₁₋₅haloalkyl. In some embodiments, the C₁₋₅alkyl is a C₁₋₅fluoroalkyl.

In some embodiments, the C₁₋₅alkyl is selected from the group consisting of —CF₂CF₃, —CF₂CH₃, —CF₂CH₂OH, —CF₂C(CH₃)₂OH, —CHF₂, —CF₃, and —CH₂F.

In some embodiments of Formula (I),

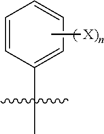

is selected from the group consisting of:

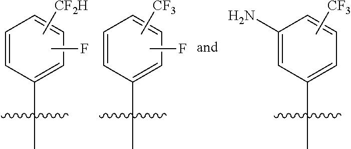

In some embodiments of Formula (I),

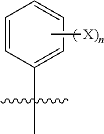

is selected from the group consisting of:

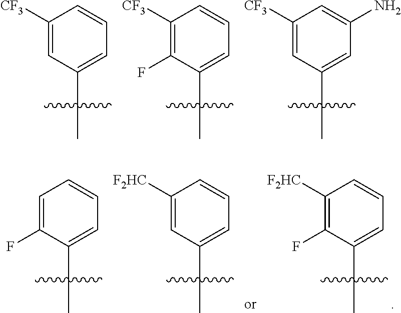

In some embodiments of Formula (I),

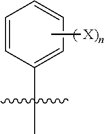

is selected from the group consisting of:

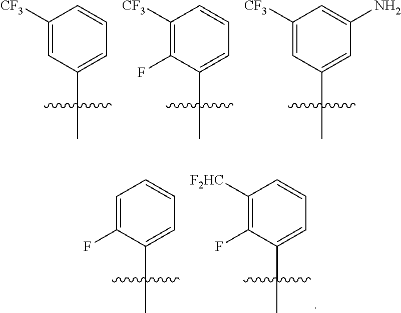

In some embodiments, L₁ and L₂ are each independently absent, or a linking group selected from the group consisting of alkylene, alkylene-O—, alkylene-N(R_{B})—, —O-alkylene, —N(R_{B})-alkylene, cycloalkyl, —O—, and —N(R_{B})—. In some embodiments, L₁ and L₂ are each independently absent, or a linking group selected from the group consisting of alkylene, alkylene-O—, alkylene-N(R_{B})—, —O-alkylene, —N(R_{B})-alkylene, —O—, and —N(R_{B})—. In some embodiments, L₁ and L₂ are each independently absent, or a linking group selected from the group consisting of alkylene, —O— alkylene, —N(R_{B})-alkylene, —O—, and —N(R_{B})—. In some embodiments, L₁ and L₂ are each independently a linking group selected from the group consisting of alkylene, —O-alkylene, —N(R_{B})-alkylene, —O—, and —N(R_{B})—, wherein R_{B} is hydrogen, alkyl, or alkylenecycloalkyl. In some embodiments, L₁ and L₂ are each independently absent, —O— or —N(R_{B})—, wherein R_{B} is hydrogen, alkyl, or alkylenecycloalkyl. In some embodiments, L₁ and L₂ are each independently —O— or —N(R_{B})—, wherein R_{B} is hydrogen, alkyl, or alkylenecycloalkyl. In some embodiments, L₁ and L₂ are each —O—. In some embodiments, L₁ is a linking group selected from the group consisting of alkylene, —O-alkylene, —N(R_{B})-alkylene, —O—, and —N(R_{B})—, wherein R_{B} is hydrogen, alkyl, or alkylenecycloalkyl and L₂ is absent or —O—. In some embodiments, L₁ is a linking group selected from the group consisting of alkylene, —O-alkylene, —N(R_{B})-alkylene, —O—, and —N(R_{B})—, wherein R_{B} is hydrogen, alkyl, or alkylenecycloalkyl and L₂ is absent. In some embodiments, L₁ is alkylene or —O— and L₂ is absent or —O—. In some embodiments, L₁ is alkylene and L₂ is absent or —O—. In some embodiments, L₂ is a linking group selected from the group consisting of alkylene, —O— alkylene, —N(R_{B})-alkylene, —O—, and —N(R_{B})—, wherein R_{B} is hydrogen, alkyl, or alkylenecycloalkyl and L₁ is absent. In some embodiments, R_{B} is hydrogen, alkyl (e.g., C₁₋₅ alkyl, C₁₋₃ alkyl, and the like), or alkylenecycloalkyl (e.g., —CH₂cyclopropyl, —CH₂cyclobutyl, —CH₂cyclopentyl, —CH₂cyclohexyl, and the like). In some embodiments, L₁ is —O— or —N(R_{B})—, wherein R_{B} is hydrogen, alkyl, or alkylenecycloalkyl and L₂ is absent. In some embodiments, L₁ is —O— and L₂ is absent. In some embodiments, L₂ is —O— or —N(R_{B})—, wherein R_{B} is hydrogen, alkyl, or alkylenecycloalkyl and L₁ is absent. In some embodiments, L₂ is —O— and L₁ is absent. In some embodiments, the alkylene is a C₁₋₅alkylene. In some embodiments, the alkylene is a C₁₋₃alkylene. In some embodiments, the alkylene is —CH₂— or —CH₂CH₂—. In some embodiments, the alkylene is —CH₂—. In some embodiments, the alkylene is —CH₂CH₂—. In some embodiments, the alkylene is —CH₂CH₂CH₂—. In some embodiments, the alkylene is substituted with one or more halogens (e.g., F, C₁, and/or Br) and/or one or more alkyl groups (e.g., —CH₃, —CH₂CH₃, —CH₂CH₂CH₃, and the like). In some embodiments, the alkylene is gem-disubstituted. In some embodiments, the alkylene is gem-disubstituted with two halogens as defined herein. In some embodiments, the alkylene is gem-disubstituted with two alkyl groups as defined herein. In some embodiments, two alkyl groups taken together with the atoms to which they are attached form a C₃₋₆cycloalkyl. In some embodiments, two alkyl groups taken together with the atoms to which they are attached form a cyclopropyl. In some embodiments, the alkylene comprises one or more —CF₂, —CHF, —C(H)(CH)₃—, —C(CH₃)₂— and

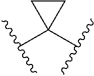 [SMILES: *C1(*)CC1]

groups. In some embodiments, R_{B} is hydrogen, C₁₋₅alkyl, or C₁₋₃alkylene-(C₃₋₆cycloalkyl). In some embodiments, R_{B} is H or C₁₋₅alkyl. In some embodiments, R_{B} is C₁₋₅alkyl or C₁₋₃alkylene-(C₃₋₆cycloalkyl)

In some embodiments of Formula (I), R₁ and R₂ are independently hydrogen or alkyl (e.g., methyl, ethyl, n-propyl, isopropyl, n-butyl, isobutyl, t-butyl, n-pentyl, isoamyl, neopentyl, and the like), wherein at least one of R₁ and R₂ is not hydrogen. In some embodiments, R₁ and R₂ are independently hydrogen or C₁₋₅ alkyl, wherein at least one of R₁ and R₂ is not hydrogen. In some embodiments, R₁ and R₂ are independently hydrogen or methyl, wherein at least one of R₁ and R₂ is not hydrogen. In some embodiments, R₁ is methyl and R₂ is H. In some embodiments, R₁ and R₂ together with the atom to which they are attached form a cycloalkyl or heterocyclyl. In some embodiments, R₁ and R₂ together with the atom to which they are attached form a cycloalkyl. In some embodiments, the cycloalkyl is a C₃₋₈ cycloalkyl. In some embodiments, the cycloalkyl is a C₃_₆ cycloalkyl. In some embodiments, R₁ and R₂ together with the atom to which they are attached form a cyclopropyl.

In some embodiments of Formula (I), R₃ and R₄ are independently absent (i.e., when [symbol] is a double bond), hydrogen, alkyl, —(C═O)—OR_{A}, —(C═O)—N(R_{A})₂, cycloalkyl, heterocyclyl, aryl or heteroaryl. In some embodiments, R₃ and R₄ are independently absent, hydrogen, alkyl, or halogen. In some embodiments, R₃ and R₄ are independently absent, hydrogen or alkyl. In some embodiments, R₃ and R₄ are independently absent, hydrogen, alkyl, halogen, —(C═O)—OR_{A}, or —(C=O)—N(R_{A})₂. In some embodiments, the alkyl is a C₁₋₅alkyl. In some embodiments, the —C₁₋₅ alkyl is methyl, ethyl, n-propyl, isopropyl, n-butyl, t-butyl, isoamyl or neopentyl. In some embodiments, the C₁₋₅alkyl is methyl. In some embodiments, the cycloalkyl is a C₃₋₈ cycloalkyl. In some embodiments, the cycloalkyl is cyclopropyl. In some embodiments, the heterocyclyl is a 4- to 12-member heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the aryl is a phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, R_{A} is selected from the group consisting of hydrogen and alkyl. In some embodiments, the alkyl is a C₁₋₅alkyl. In some embodiments, the C₁₋₅ alkyl is methyl, ethyl, or isopropyl. In some embodiments, R₃ and R₄ are each hydrogen. In some embodiments, R₃ and R₄ together form a carbonyl. In some embodiments, R₃ is hydrogen, alkyl, —(C═O)—OR_{A}, —(C═O)—N(R_{A})₂, cycloalkyl, heterocyclyl, aryl or heteroaryl and R₄ is H or absent. In some embodiments, R₃ is hydrogen, alkyl, —(C═O)—OR_{A}, or —(C═O)—N(R_{A})₂ and R₄ is H or absent. In some embodiments, R₃ is alkyl, —(C═O)—OR_{A}, or —(C═O)—N(R_{A})₂ and R₄ is H or absent. In some embodiments, R₃ is selected from the group consisting of hydrogen, alkyl, —(C═O)—OCH₃, —(C═O)—OH, and —(C═O)—NH₂ and R₄ is H or absent. In some embodiments, R₃ is hydrogen or alkyl and R₄ is H or absent. In some embodiments, R₃ is selected from the group consisting of methyl, ethyl, isopropyl, n-propyl, —CH₂OH, —CH₂OCH₃, —CH₂N(CH₃)₂, —CH(OH)(CH₃)₂ and —CH₂(OH)CH₃ and R₄ is H or absent. In some embodiments, R₃ is H or methyl and R₄ is H or absent. In some embodiments, R₃ is H and R₄ is H or absent. In some embodiments, R₃ is methyl and R₄ is H or absent.

In some embodiments of Formula (I), [symbol] is a double bond and R₃ is hydrogen, alkyl, —O-alkyl, —(C═O)—OR_{A}, —(C═O)—N(R_{A})₂, cycloalkyl, heterocyclyl, aryl or heteroaryl. In some embodiments, R₃ is hydrogen, alkyl, or halogen. In some embodiments, R₃ is hydrogen or alkyl. In some embodiments, R₃ is hydrogen, alkyl, halogen, —(C═O)—OR_{A}, or —(C═O)—N(R_{A})₂. In some embodiments, the alkyl is a C₁₋₅alkyl. In some embodiments, the —C₁₋₅ alkyl is methyl, ethyl, n-propyl, isopropyl, n-butyl, t-butyl, isoamyl or neopentyl. In some embodiments, the C₁₋₅alkyl is methyl. In some embodiments, the cycloalkyl is a C₃₋₈ cycloalkyl. In some embodiments, the cycloalkyl is cyclopropyl. In some embodiments, the heterocyclyl is a 4- to 12-member heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the aryl is a phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, R_{A} is selected from the group consisting of hydrogen and alkyl. In some embodiments, the alkyl is a C₁₋₅alkyl. In some embodiments, the C₁₋₅ alkyl is methyl, ethyl, or isopropyl. In some embodiments, R₃ is hydrogen or alkyl. In some embodiments, R₃ is selected from the group consisting of methyl, ethyl, isopropyl, n-propyl, —CH₂OH, —CH₂OCH₃, —CH₂N(CH₃)₂, —CH(OH)(CH₃)₂ and —CH₂(OH)CH₃. In some embodiments, R₃ is H or methyl. In some embodiments, R₃ is H. In some embodiments, R₃ is methyl.

In some embodiments of Formula (I), [symbol] is a double bond. In some embodiments, [symbol] is a single bond. In some embodiments, [symbol] is a single bond and R₃ and R₄ together form a carbonyl (i.e., oxo group). In some embodiments, [symbol] is a single bond and R₃ and R₄ taken together are not a carbonyl.

In some embodiments, [symbol] is a single bond and R₅ is alkyl or alkylenecycloalkyl. In some embodiments, [symbol] is a single bond and R₅ is alkyl In some embodiments, the alkyl is a C₁₋₅alkyl. In some embodiments, the C₁₋₅alkyl is methyl, ethyl, or isopropyl. In some embodiments, the alkylenecycloalkyl is a C₁₋₃alkylene-(C₃₋₆cycloalkyl).

In some embodiments of Formula (I), R₆ is alkyl, cycloalkyl, cycloalkenyl, heterocyclyl, aryl, or heteroaryl. In some embodiments of Formula (I), R₆ is alkyl, cycloalkyl, heterocyclyl, aryl, or heteroaryl. In some embodiments, R₆ is cycloalkyl, cycloalkenyl, heterocyclyl, aryl, or heteroaryl. In some embodiments, R₆ is cycloalkyl, heterocyclyl, aryl, or heteroaryl. In some embodiments, R₆ is alkyl, heterocyclyl, cycloalkyl, or cycloalkenyl. In some embodiments, R₆ is heterocyclyl or cycloalkyl. In some embodiments, the alkyl is a C₁₋₅alkyl. In some embodiments, the C₁₋₅alkyl is methyl, ethyl, n-propyl, isopropyl, n-butyl, t-butyl, isoamyl or neopentyl. In some embodiments, the C₁₋₅alkyl is methyl. In some embodiments, the cycloalkyl is a C₃₋₈cycloalkyl. In some embodiments, the cycloalkyl is a C₃₋₆cycloalkyl. In some embodiments, the cycloalkyl is cyclobutyl, cyclopentyl or cyclohexyl. In some embodiments, the cycloalkyl is cyclopentyl. In some embodiments, the heterocyclyl is a 4- to 12-membered heterocyclyl with 1 or 2 heteroatoms selected from N, O, and S. In some embodiments, the cycloalkyl is cyclopentyl. In some embodiments, the heterocyclyl is a 4- to 12-membered heterocyclyl with 1 or 2 heteroatoms selected from N, O, and S substituted with one or two oxo groups. In some embodiments, the heterocyclyl is a 5- or 6-membered heterocyclyl comprising a heteroatom selected from N, O, and S. In some embodiments, the heterocyclyl is a morpholinyl, piperazinyl, piperidinyl, pyrrolidinyl, azetidinyl, tetrahydropyranyl, or tetrahydrofuranyl. In some embodiments, the heterocyclyl is a lactam, e.g.,

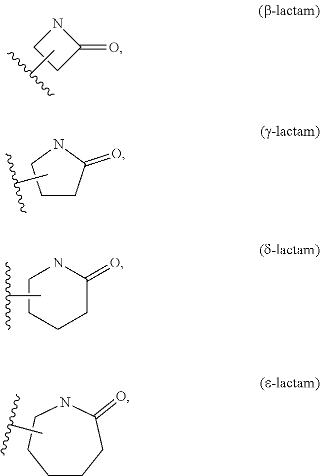

and the like. In some embodiments, the heterocyclyl is 3-tetrahydrofuranyl or 3-tetrahydropyranyl. In some embodiments, the aryl is a phenyl. In some embodiments, the heteroaryl is a 5- to 14-membered heteroaryl having 1, 2, or 3 heteroatoms selected from N, O, and S. In some embodiments, the 5- to 14-membered heteroaryl is selected from the group consisting of pyrazolyl, pyridinyl, pyrimidinyl, pyridazinyl, pyrazinyl, imidazolyl, oxazolyl, thiazolyl, oxadiazole, thiadiazolyl, triazolyl, thiophene, benztriazolyl, benzoxazolyl, benzthiazolyl, benzimidazolyl, quinolinyl, isoquinolinyl, and cinnolinyl. In some embodiments, the heteroaryl is a 5- or 6-membered heteroaryl having 1, 2, or 3 heteroatoms selected from N, O, and S. In some embodiments, the heteroaryl is pyrazolyl, pyridinyl, pyrimidinyl, imidazolyl, oxazolyl, thiazolyl, oxadiazole, thiadiazolyl, or triazolyl. In some embodiments, the heteroaryl is pyrazolyl, pyridinyl, pyrimidinyl, imidazolyl, oxazolyl, or thiazolyl. In some embodiments, R₆ is methyl, ethyl, n-propyl, or isopropyl. In some embodiments, R₆ is methyl. In some embodiments, R₆ is 3-tetrahydrofuranyl or 3-tetrahydropyranyl. In some embodiments, R₆ is 3-tetrahydrofuranyl or cyclopentyl. In some embodiments, R₆ is 3-tetrahydrofuranyl. In some embodiments, R₆ is cyclopentyl. In some embodiments, R₆ is H.

In some embodiments, R₆ is:

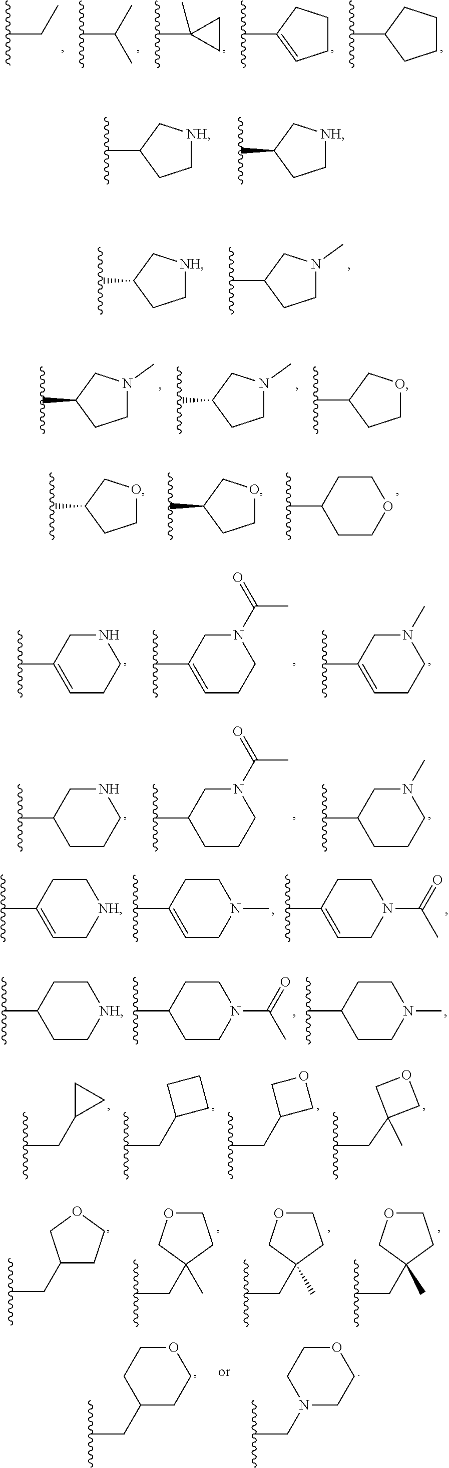

In some embodiments of Formula (I), R₇ is halogen, alkyl, cycloalkyl, heterocyclyl, aryl, or heteroaryl. In some embodiments, R₇ is halogen, alkyl, or cycloalkyl, or heterocyclyl. In some embodiments, R₇ is cycloalkyl, heterocyclyl, aryl, or heteroaryl. In some embodiments, R₇ is heterocyclyl or cycloalkyl. In some embodiments, the halogen is F, Cl, or Br. In some embodiments, the alkyl is a C₁₋₅alkyl. In some embodiments, the C₁₋₅alkyl is methyl, ethyl, n-propyl, isopropyl, n-butyl, t-butyl, isoamyl or neopentyl. In some embodiments, the C₁₋₅alkyl is methyl. In some embodiments, the C₁₋₅alkyl is C₁₋₅haloalkyl. In some embodiments, the haloalkyl is —CF₃, —CF₂H, —CFH₂, —CF₂CF₃, or —CH₂CF₃. In some embodiments, the haloalkyl is —CF₃. In some embodiments, the cycloalkyl is a C₃₋₈cycloalkyl. In some embodiments, the cycloalkyl is a C₃₋₆cycloalkyl. In some embodiments, the cycloalkyl is cyclobutyl, cyclopentyl or cyclohexyl. In some embodiments, the cycloalkyl is cyclopentyl. In some embodiments, the heterocyclyl is a 4- to 12-membered heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the heterocyclyl is 3-tetrahydrofuranyl or 3-tetrahydropyranyl. In some embodiments, the aryl is a phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, R₇ is H, halogen, C₁₋₅alkyl, C₃₋₆cycloalkyl, C₄₋₆heterocyclyl, or 5- to 6-membered heteroaryl. In some embodiments, R₇ is a C₄₋₆heterocyclyl. In some embodiments, R₇ is a C₃₋₆cycloalkyl. In some embodiments, R₇ is a 5- or 6 membered heteroaryl. In some embodiments, R₇ is a C₁₋₅alkyl. In some embodiments, R₇ is methyl, ethyl, n-propyl, or isopropyl. In some embodiments, R₇ is H. In some embodiments, R₇ is methyl. In some embodiments, R₇ is 3-tetrahydrofuranyl or 3-tetrahydropyranyl. In some embodiments, R₇ is 3-tetrahydrofuranyl or cyclopentyl. In some embodiments, R₇ is 3-tetrahydrofuranyl. In some embodiments, R₇ is cyclopentyl. In some embodiments, R₇ is F, Cl, or Br. In some embodiments, R₇ is F or Cl. In some embodiments, R₇ is F.

In some embodiments of Formula (I), L₁-R₆ and L₂-R₇ are each independently H, alkyl, cycloalkyl, cycloalkenyl, alkylenecycloalkyl, alkylenecycloalkenyl, —O-alkyl, —O-cycloalkyl, —O-cycloalkenyl, —O-heterocyclyl, —O-aryl, —O-heteroaryl, —N(R_{B})-alkyl, —N(R_{B})-cycloalkyl, —N(R_{B})-heterocyclyl, —N(R_{B})-aryl, or —N(R_{B})-heteroaryl, provided that at least one of L₁-R₆ and L₂-R₇ is not H. In some embodiments of Formula (I), L₁-R₆ and L₂-R₇ are each independently H, alkyl, cycloalkyl, alkylenecycloalkyl, —O-alkyl, —O-cycloalkyl, —O-heterocyclyl, —O-aryl, —O— heteroaryl, —N(R_{B})-alkyl, —N(R_{B})-cycloalkyl, —N(R_{B})-heterocyclyl, —N(R_{B})-aryl, or —N(R_{B})-heteroaryl, provided that at least one of L₁-R₆ and L₂-R₇ is not H. In some embodiments, L₁-R₆ and L₂-R₇ are each independently H, alkyl, cycloalkyl, alkylenecycloalkyl, —O-alkyl, —O— cycloalkyl, —O-heterocyclyl, —O-aryl, or —O-heteroaryl, provided that at least one of L₁-R₆ and L₂-R₇ is not H. In some embodiments, L₁-R₆ and L₂-R₇ are each independently H, alkyl, cycloalkyl, cycloalkenyl, alkylenecycloalkyl, alkylenecycloalkenyl, —O-alkyl, —O-cycloalkyl, or —O-heterocyclyl, provided that at least one of L₁-R₆ and L₂-R₇ is not H. In some embodiments, L₁-R₆ and L₂-R₇ are each independently H, alkyl, cycloalkyl, alkylenecycloalkyl, —O-alkyl, —O— cycloalkyl, or —O-heterocyclyl, provided that at least one of L₁-R₆ and L₂-R₇ is not H. In some embodiments, the alkylene is a C₁₋₃alkylene. In some embodiments, the alkylene is methylene (—CH₂—) or ethylene (—CH₂CH₂—). In some embodiments, L₁-R₆ and L₂-R₇ are each independently H, —O-cycloalkyl, —O-heterocyclyl, —O-aryl, or —O-heteroaryl, provided that at least one of L₁-R₆ and L₂-R₇ is not H. In some embodiments, the —O-alkyl is a —O—C₁₋₅ alkyl. In some embodiments, the —O—C₁₋₅ alkyl is —O-methyl, —O-ethyl, —O-n-propyl, —O-isopropyl, —O-n-butyl, —O-t-butyl, —O-isoamyl or —O-neopentyl. In some embodiments, the —O—C₁₋₅ alkyl is —O-methyl. In some embodiments, the —O-cycloalkyl is a —O—C₃₋₈ cycloalkyl. In some embodiments, the —O-cycloalkyl is a —O-cyclopentyl. In some embodiments, the heterocyclyl is a 4- to 12-member heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the heterocyclyl is 3-tetrahydrofuranyl or 3-tetrahydropyranyl. In some embodiments, the —O-aryl is a —O-phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, L₁-R₆ and L₂-R₇ are each independently H, —O-methyl, —O-ethyl, —O-n-propyl, —O-isopropyl, —O-cyclopentyl, —O-3-tetrahydrofuranyl, or —O-3-tetrahydropyranyl, provided that at least one of L₁-R₆ and L₂-R₇ is not H. In some embodiments, one L₁-R₆ and L₂-R₇ is —O-methyl. In some embodiments, one of L₁-R₆ and L₂-R₇ is —O-3-tetrahydrofuranyl or —O-3-tetrahydropyranyl. In some embodiments, one of L₁-R₆ and L₂-R₇ is —O-3-tetrahydrofuranyl.

In some embodiments, R₈ is H, halogen, alkyl, alkoxy, or —CH₂—O-alkyl. In some embodiments, R₈ is H, halogen, C₁₋₅alkyl, C₁_alkoxy, or —CH₂—O—C₁₋₅alkyl. In some embodiments, R₈ is halogen or C₁₋₅alkyl. In some embodiments, R₈ is C₁₋₅alkyl. In some embodiments, the C₁₋₅alkyl is methyl. In some embodiments, R₈ is halogen. In some embodiments, the halogen is F or Cl. In some embodiments, the C₁₋₅alkoxy is methoxy.

In some embodiments, R₉ is H, alkyl, cycloalkyl, heterocyclyl, alkylene-cycloalkyl, or alkylene-heterocyclyl. In some embodiments, R₉ is H or alkyl. In some embodiments, R₉ is H or methyl. In some embodiments, R₉ is H. In some embodiments, R₉ is methyl.

In some embodiments of Formula (I),

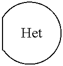

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is alkyl, cycloalkyl, cycloalkenyl, heterocyclyl, aryl, or heteroaryl; and R₇ is H, halogen, alkyl, cycloalkyl, heterocyclyl, aryl, or heteroaryl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, C₁₋₅alkyl, C₃₋₆cycloalkyl, 4- to 7-membered heterocyclyl, phenyl, or 5- to 6-membered heteroaryl.

In some embodiments of Formula (I),

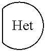

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; L₁ is absent, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is alkyl, cycloalkyl, heterocyclyl, aryl, or heteroaryl; and R₇ is H, halogen, alkyl, cycloalkyl, heterocyclyl, aryl, or heteroaryl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, C₁₋₅alkyl, C₃₋₆cycloalkyl, 4- to 7-membered heterocyclyl, phenyl, or 5- to 6-membered heteroaryl.

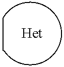

In some embodiments of Formula (I), is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; L₁ is absent, alkylene, or —O—; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is alkyl, cycloalkyl, heterocyclyl, aryl, or heteroaryl; and R₇ is H, halogen, alkyl, cycloalkyl, heterocyclyl, aryl, or heteroaryl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, C₁₋₅alkyl, C₃₋₆cycloalkyl, 4- to 7-membered heterocyclyl, phenyl, or 5- to 6-membered heteroaryl.

In some embodiments of Formula (I),

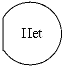

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl; L₂ is absent or —O—; R₃ is hydrogen or alkyl; R₄ is absent; R₅ is absent; R₆ is alkyl, cycloalkyl, heterocyclyl, aryl, or heteroaryl; and R₇ is H, halogen, alkyl, cycloalkyl, heterocyclyl, aryl, or heteroaryl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, C₁₋₅alkyl, C₃₋₆cycloalkyl, 4- to 7-membered heterocyclyl, phenyl, or 5- to 6-membered heteroaryl.

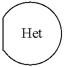

In some embodiments of Formula (I), is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, or —O—; L₂ is absent or —O—; R₃ is hydrogen or alkyl; R₄ is absent; R₅ is absent; R₆ is alkyl, cycloalkyl, heterocyclyl, aryl, or heteroaryl; and R₇ is H, halogen, alkyl, cycloalkyl, heterocyclyl, aryl, or heteroaryl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, C₁₋₅alkyl, C₃₋₆cycloalkyl, 4- to 7-membered heterocyclyl, phenyl, or 5- to 6-membered heteroaryl.

In some embodiments of Formula (I),

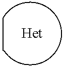

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is alkyl, cycloalkyl, cycloalkenyl, or heterocyclyl; and R₇ is H, halogen, or alkyl. In some embodiments, R₆ is C₁₋₅alkyl, C₃₋₆cycloalkyl, 4- to 7-membered heterocyclyl, phenyl, or 5- to 6-membered heteroaryl. In some embodiments, R₇ is H, F, or C₁₋₅alkyl.

In some embodiments of Formula (I),

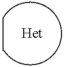

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is cycloalkyl or heterocyclyl; and R₇ is H, halogen, or alkyl. In some embodiments, R₆ is C₃₋₆cycloalkyl or 4- to 7-membered heterocyclyl. In some embodiments, R₇ is H, F, or C₁₋₅alkyl.

In some embodiments of Formula (I),

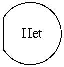

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; L₁ is absent, alkylene, or —O—; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is alkyl, cycloalkyl, cycloalkenyl, or heterocyclyl; and R₇ is H, halogen, or alkyl. In some embodiments, R₆ is C₁₋₅alkyl, C₃₋₆cycloalkyl, C₅₋₆cycloalkenyl; or 4- to 7-membered heterocyclyl. In some embodiments, R₇ is H, F, or C₁₋₅ alkyl.

In some embodiments of Formula (I),

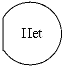

is a 6-membered heteroaryl having 1 or 2 nitrogen atoms; each X is independently selected from the group consisting of halogen, C₁₋₅alkyl, —NH₂, and C₁₋₅alkoxy; n is an integer from 1-3; R₁ and R₂ are each independently H or C₁₋₅alkyl; Li is absent, alkylene, or —O—; L₂ is absent or —O—; R₃ is selected from the group consisting of hydrogen, alkyl, —(C═O)—OR_{A}, —(C═O)—N(R_{A})₂, cycloalkyl, heterocyclyl, aryl or heteroaryl, wherein R_{A} is as defined herein; R₄ is absent; R₅ is absent; R₆ is cycloalkyl or heterocyclyl; and R₇ is H, halogen, or alkyl. In some embodiments, R₆ is C₃₋₆cycloalkyl or 4- to 7-membered heterocyclyl. In some embodiments, R₇ is H, F, or C₁₋₅alkyl.

In another aspect, the present disclosure provides a compound of Formula (Ic):

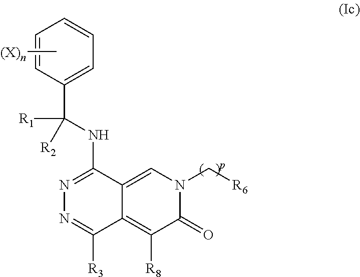

or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen;
R₃ is absent, hydrogen, alkyl, —(C═O)—OR_{A}, —(C═O)—N(R_{A})₂, cycloalkyl, heterocyclyl, aryl or heteroaryl, wherein R_{A} is selected from the group consisting of hydrogen and alkyl;
R₆ is H, alkyl, cycloalkyl, heterocyclyl, aryl or heteroaryl;
R₈ is H, halogen, alkyl, alkoxy, alkylene-O-alkyl, cycloalkyl, or heterocyclyl;
X is independently selected from the group consisting of C₁₋₅ alkyl, F, CF₃, CHF₂, CH₂F, and —NH₂, and n is an integer from 1-5; and
p is 0-3.

In some embodiments, R₃ is hydrogen, alkyl, —O-alkyl, —(C═O)—OR_{A}, —(C═O)—N(R_{A})₂, cycloalkyl, heterocyclyl, aryl or heteroaryl. In some embodiments, R₃ is hydrogen, alkyl, or halogen. In some embodiments, R₃ is hydrogen or alkyl. In some embodiments, R₃ is hydrogen, alkyl, halogen, —(C═O)—OR_{A}, or —(C═O)—N(R_{A})₂. In some embodiments, the alkyl is a C₁₋₅alkyl. In some embodiments, the —C₁₋₅ alkyl is methyl, ethyl, n-propyl, isopropyl, n-butyl, t-butyl, isoamyl or neopentyl. In some embodiments, the C₁₋₅alkyl is methyl. In some embodiments, the cycloalkyl is a C₃₋₈ cycloalkyl. In some embodiments, the cycloalkyl is cyclopropyl. In some embodiments, the heterocyclyl is a 4- to 12-member heterocyclyl with 1 or 2 heteroatoms selected from N, O, and S. In some embodiments, the heterocyclyl is a 5- or 6-membered heterocyclyl comprising a heteroatom selected from N, O, and S. In some embodiments, the aryl is a phenyl. In some embodiments, the heteroaryl is a 5- to 14-membered heteroaryl having 1, 2, or 3 heteroatoms selected from N, O, and S. In some embodiments, the heteroaryl is a 5- or 6-membered heteroaryl having 1, 2, or 3 heteroatoms selected from N, O, and S. In some embodiments, R_{A} is selected from the group consisting of hydrogen and alkyl. In some embodiments, the alkyl is a C₁₋₅alkyl. In some embodiments, the C₁₋₅ alkyl is methyl, ethyl, or isopropyl. In some embodiments, R₃ is hydrogen or alkyl and R₄ is absent. In some embodiments, R₃ is selected from the group consisting of methyl, ethyl, isopropyl, n-propyl, —CH₂OH, —CH₂OCH₃, —CH₂N(CH₃)₂, —CH(OH)(CH₃)₂ and —CH₂(OH)CH₃. In some embodiments, R₃ is hydrogen.

In some embodiments, the present disclosure provides a compound of Formula (Ic-1):

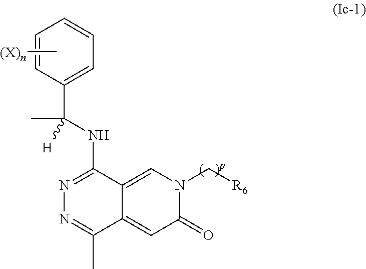

or a pharmaceutically acceptable salt or stereoisomer thereof, wherein: X, R₆, and n, and p is an integer from 0-3.

In some embodiments, the present disclosure provides a compound of Formula (Ic-2):

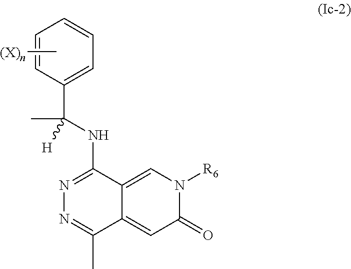

or a pharmaceutically acceptable salt or stereoisomer thereof, wherein: X, R₆, and n are as defined herein.

In some embodiments, the present disclosure provides a compound of Formula (Id):

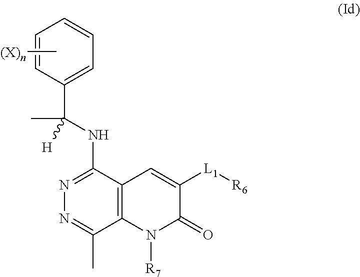

or a pharmaceutically acceptable salt or stereoisomer thereof, wherein: X, L₁, R₆, R₇, and n are as defined herein.

In some embodiments, the present disclosure provides a compound of Formula (Id):

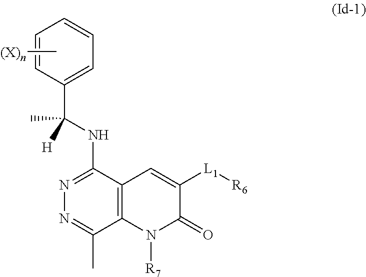

or a pharmaceutically acceptable salt or stereoisomer thereof, wherein: X, L₁, R₆, R₇, and n are as defined herein.

In some embodiments, the compound of Formula (Id) and Formula (Id-1) is not:

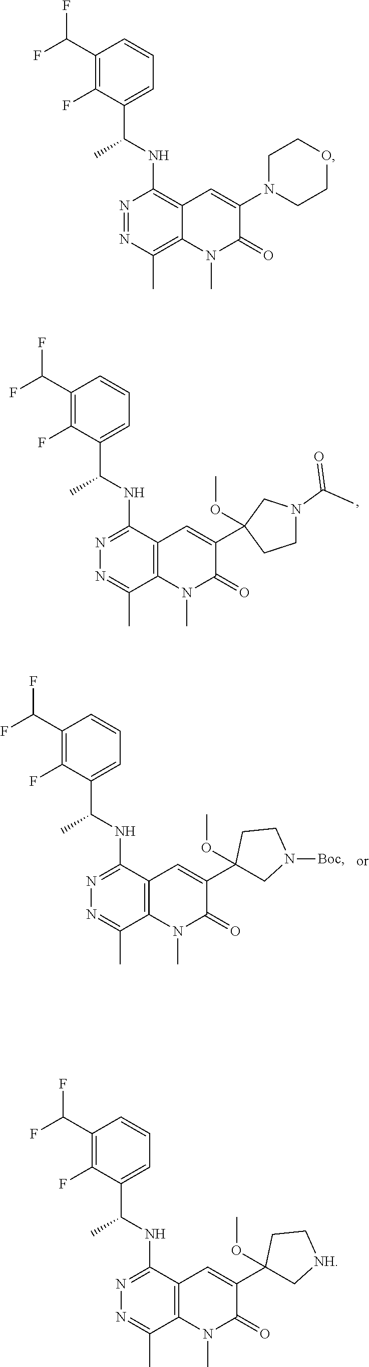 [SMILES: Cc1nnc(N[C@H](C)c2cccc(C(F)F)c2F)c2cc(N3CCOCC3)c(=O)n(C)c12 ; COC1(c2cc3c(N[C@H](C)c4cccc(C(F)F)c4F)nnc(C)c3n(C)c2=O)CCN(C(C)=O)C1 ; COC1(c2cc3c(N[C@H](C)c4cccc(C(F)F)c4F)nnc(C)c3n(C)c2=O)CCN(C(=O)OC(C)(C)C)C1 ; COC1(c2cc3c(N[C@H](C)c4cccc(C(F)F)c4F)nnc(C)c3n(C)c2=O)CCNC1]

In some embodiments, the present disclosure provides a compound of Formula (Ie) or (If):

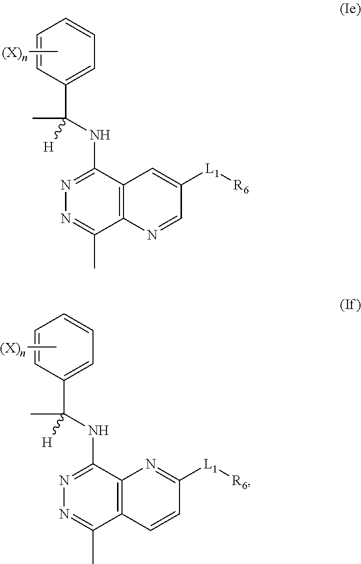

or a pharmaceutically acceptable salt or stereoisomer thereof, wherein: X, L₁, R₆, and n are as defined herein.

In some embodiments, the compound of the present disclosure is selected from the group consisting of:

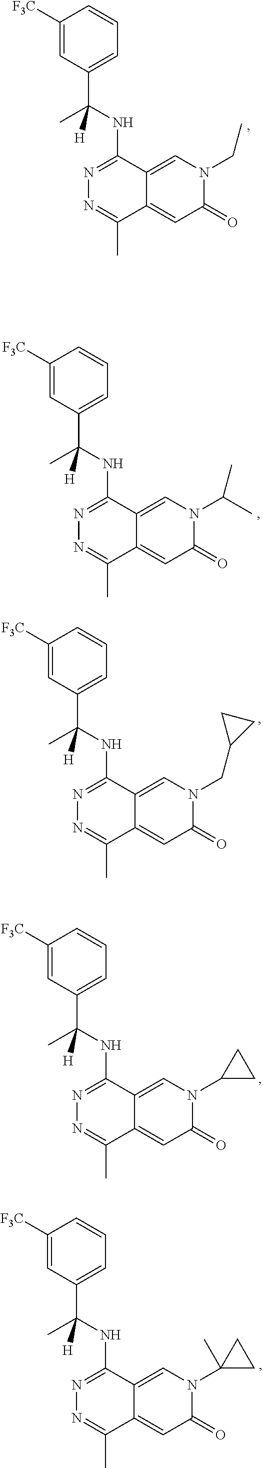

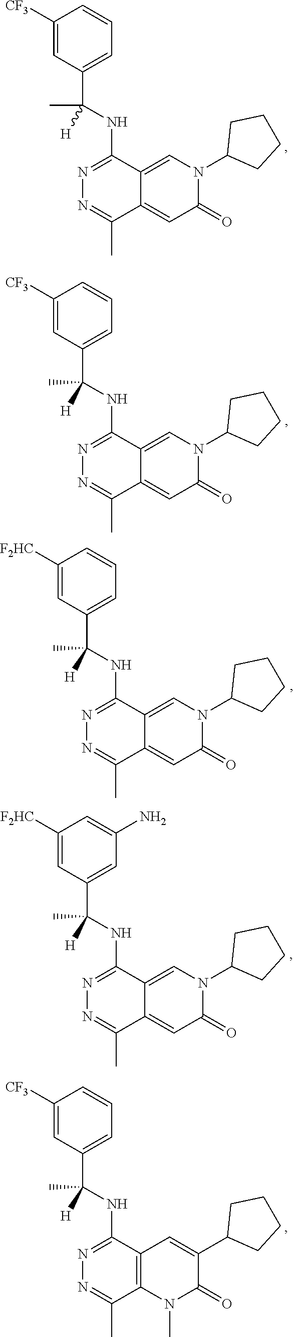

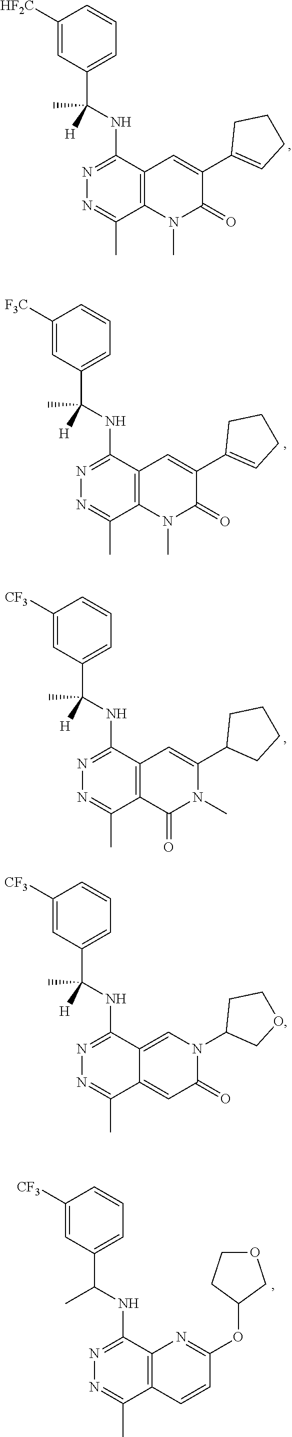

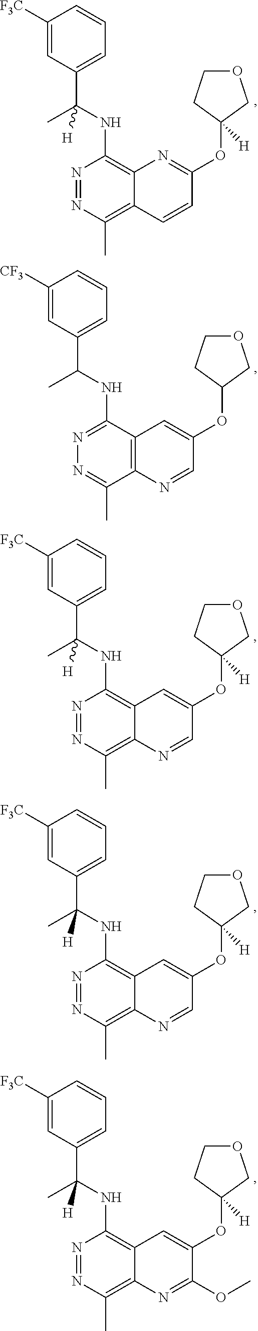

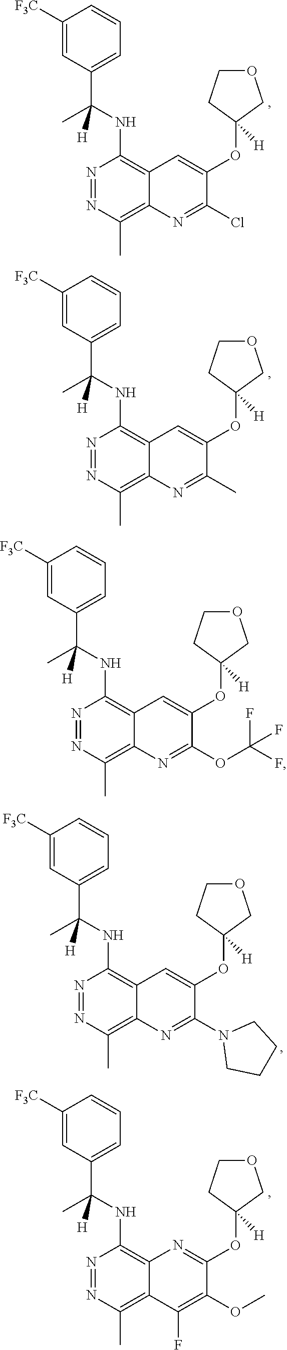

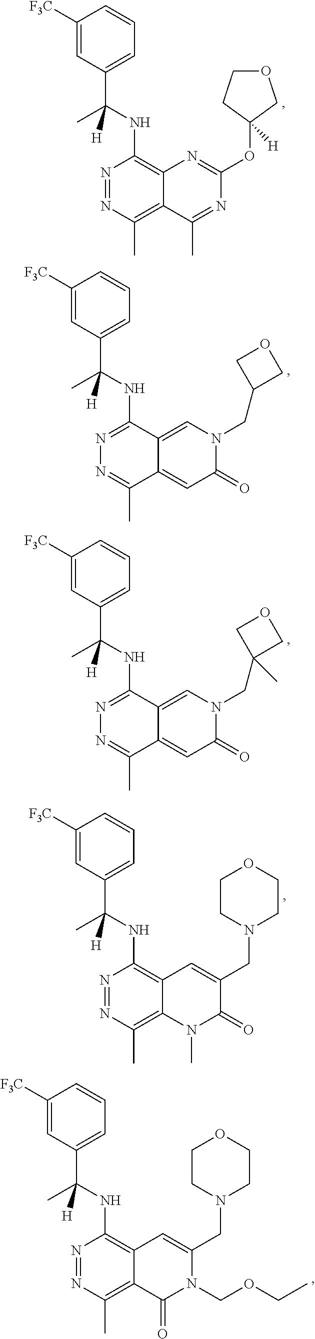

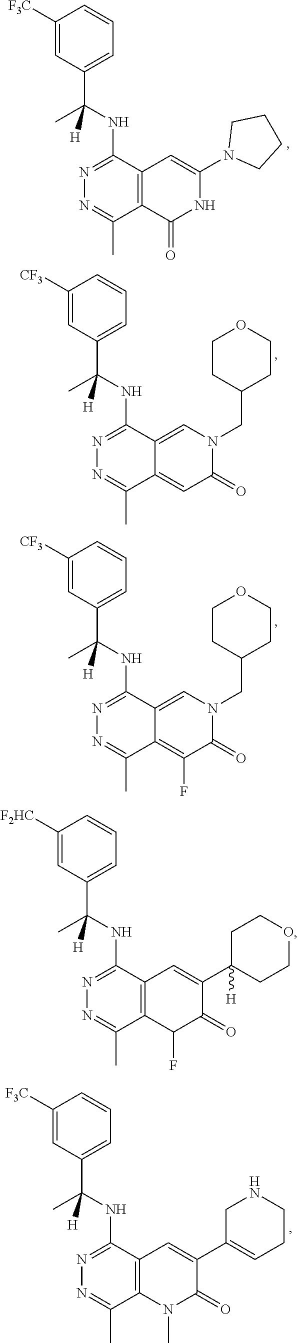

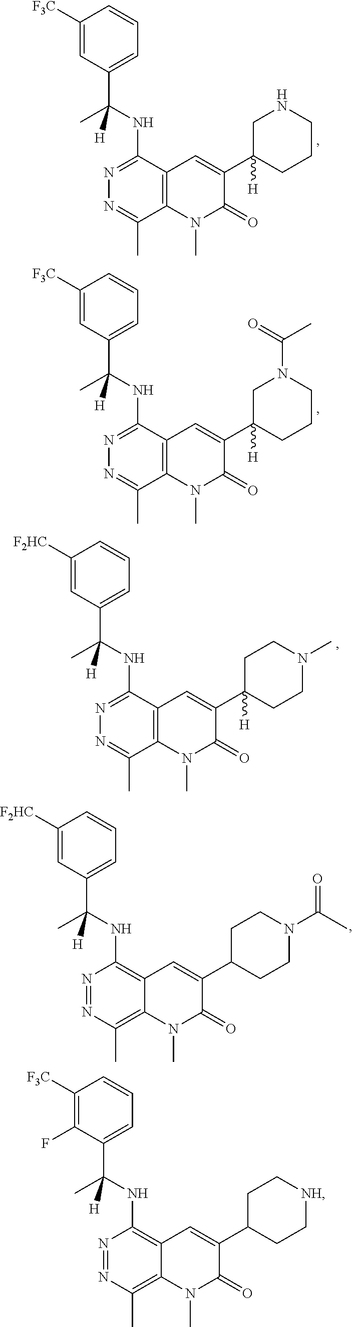

and a pharmaceutically acceptable salt thereof.

In some embodiments, the compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)) is a compound of Table 3.

In some embodiments, disclosed herein are compounds of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If), wherein the formulas disclosed herein exclude the compounds described in WO2021/127429, WO2022/017339, WO2022/251497, WO2022/184116, and WO2022/156792.

### Pharmaceutical Compositions

In various embodiments, the present disclosure provides a pharmaceutical composition comprising a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)) or a pharmaceutically acceptable salt thereof, and a pharmaceutically acceptable excipient.

In various embodiments, the present disclosure provides a pharmaceutical composition comprising a therapeutically effective amount of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)) or a pharmaceutically acceptable salt thereof, and a pharmaceutically acceptable excipient.

In some embodiments, the pharmaceutically acceptable salt is a salt of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods of Treatment

The present disclosure is directed, in-part, to SOS1 inhibitor compounds of the present disclosure, which are useful in the treatment and/or prevention of a disease and/or condition associated with or modulated by SOS1, including wherein the inhibition of the interaction of SOS1 and a RAS-family protein and/or RAC1 is of therapeutic benefit for the treatment and/or prevention of cancer.

In some embodiments, the present disclosure provides a method of treating and/or preventing cancer comprising administering to a subject a therapeutically effective amount of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Je), or Formula (If)), a pharmaceutically acceptable salt thereof, or a pharmaceutical composition thereof.

In some embodiments, the compound of the present disclosure or pharmaceutically acceptable salt thereof is an inhibitor of SOS1.

In some embodiments, the present disclosure provides a method of treating and/or preventing a disease by inhibiting the interaction of SOS1 and a RAS-family protein or RAC1, the method comprising administering to a subject a therapeutically effective amount of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)), a pharmaceutically acceptable salt thereof, or a pharmaceutical composition thereof.

In some embodiments, the present disclosure provides a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)), a pharmaceutically acceptable salt thereof, or a pharmaceutical composition thereof, for use in a method of treating and/or preventing a disease, such as a disease associated with or modulated by SOS1.

In some embodiments, the present disclosure provides the use of a compound disclosed herein (e.g., a compound of Formula (I), Formula (Ia), Formula (Ib), Formula (Ib-1), Formula (Ic), Formula (Ic-1), Formula (Ic-2), Formula (Id), Formula (Id-1), Formula (Ie), or Formula (If)), a pharmaceutically acceptable salt thereof, or a pharmaceutical composition thereof, for the manufacture of a medicament for treating a disease, such as a diseases associated with or modulated by SOS1.

In some embodiments, the disease is cancer. In some embodiments, the cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcoma. In some embodiments, the cancer is selected from the group consisting of pancreatic cancer, lung cancer (e.g., non-small cell lung cancer (NSCLC)), cholangiocarcinoma and colorectal cancer.

In another aspect the disease/condition/cancer to be treated/prevented with a compound of the present disclosure (e.g., a SOS1 inhibitor compound) is a disease/condition/cancer defined as exhibiting one or more of the following molecular features:
1. KRAS alterations:
   a. KRAS amplification (wild type (wt) or mutant);
   b. KRAS overexpression (wt or mutant);
   c. KRAS mutation(s):
      i. G12 mutations (e.g., G12C, G12V, G12S, G12A, G12V, G12R, G12F, G12D);
      ii. G13 mutations (e.g., G13C, G13D, G13R, G13V, G13S, G13A)
      iii. T35 mutation (e.g., T35I);
      iv. 136 mutation (e.g., I36L, I36M);
      v. E49 mutation (e.g., E49K);
      vi. Q61 mutation (e.g., Q61H, Q61R, Q61P, Q61E, Q61K, Q61L, Q61K);
      vii. K117 mutation (e.g., K117N);
      viii. A146 mutation (e.g., A146T, A146V);
2. NRAS alterations:
   a. NRAS amplification (wt or mutant);
   b. NRAS overexpression (wt or mutant);
   c. NRAS mutation(s):
      i. G12 mutations (e.g., G12A, G12V, G12D, G12C, G12S, G12R);
      ii. G13 mutation (e.g., G13V, G13D, G13R, G13S, G13C, G13A);
      iii. Q61 mutation (e.g., Q61K, Q61L, Q61H, Q61P, Q61R);
      iv. A146 mutation (e.g., A146T, A146V);
3. HRAS alterations:
   a. HRAS amplification (wt or mutant);
   b. HRAS overexpression (wt or mutant);
   c. HRAS mutation(s);
      i. G12 mutation (e.g., G12C, G12V, G12S, G12A, G12V, G12R, G12F, G12D);
      ii. G13 mutation (e.g., G13C, G13D, G13R, G13V, G13S, G13A);
      iii. Q61 mutation (e.g., Q61K, Q61L, Q61H, Q61P, Q61R);
4. EGFR alterations:
   a. EGFR amplification (wt or mutant);
   b. EGFR overexpression (wt or mutant);
   c. EGFR mutation(s)
      i. e.g., exon 20 insertion, exon 19 deletion (Del19), G719X (e.g., G719A, G719C, G719S), T790M, C₇₉₇S, T854A, L858R, L861Q, or any combination thereof;
5. BRAF alterations:
   a. BRAF amplifications
   b. BRAF overexpression
   c. BRAF mutation(s) e.g., Class 2; G464V, G469V, L597Q, K601E, or Class 3; D287H, V459L, G466V
   d. Chromosomal rearrangement involving the BRAF gene
6. ErbB2 (Her2) alterations:
   a. ErbB2 amplification;
   b. ErbB2 overexpression;
   c. ErbB2 mutation(s)
      i. e.g., R678, G309, L755, D769, D769, V777, P780, V842, R896, c.2264_2278del (L755_T759del), c.2339_2340ins (G778_P780dup), S310;
7. c-MET alterations:
   a. c-MET amplification;
   b. c-MET overexpression;
   c. c-MET mutation(s)
      i. e.g., E168, N375, Q648, A887, E908, T1010, V1088, H1112, R1166, R1188, Y1248, Y1253, M1268, D1304, A1357, P1382;
8. AXL alterations:
   a. AXL amplification;
   b. AXL overexpression;
9. BCR-ABL alterations:
   a. chromosomal rearrangements involving the ABL gene;
10. ALK alterations:
   a. ALK amplification;
   b. ALK overexpression;
   c. ALK mutation(s)
      i. e.g., 1151Tins, L1152R, C₁₁₅₆Y, F1174L, L1196M, L1198F, G1202R, S1206Y, G1269A;
   d. chromosomal rearrangements involving the ALK gene;
11. FGFR1 alterations:
   a. FGFR1 amplification;
   b. FGFR1 overexpression;
12. FGFR2 alterations:
   a. FGFR2 amplification;
   b. FGFR2 overexpression;
13. FGFR3 alterations:
   a. FGFR3 amplification;
   b. FGFR3 overexpression;
   c. chromosomal rearrangement involving the FGFR3 gene;
14. FGFR4 alterations:
   a. FGFR4 amplification
   b. FGFR4 overexpression
   c. FGFR4 mutations (e.g., N535K, V550L, V550M)
   d. Chromosomal rearrangement involving the FGFR4 gene
15. cKIT alterations:
   a. cKIT amplification
   b. cKIT overexpression
   c. cKIT mutations (e.g., exon 9 insertions, exon 11 alterations (insertion or deletion), W557R, V559D, V560D, L576P, K642E, V654A, D816V, D820Y, N822K, Y823D, A829P, R888W)
16. PDGFRA alterations:
   a. PDGFRA amplification
   b. PDGFRA overexpression
   c. PDGFRA mutations (e.g., D842V, N659Y)
17. NTRK1 alterations:
   a. chromosomal rearrangements involving the NTRK1 gene;
18. NF1 alterations:
   a. NF1 mutation(s) (e.g., R440*, I679Dfs*21, R1241*, Y2285Tfs*5, R2450*)
   b. NF1 gene deletions/microdeletions
19. RET alterations:
   a. RET amplification;
   b. RET overexpression;
   c. chromosomal rearrangements involving the RET gene
20. ROS1 alterations:
   a. ROS1 amplification;
   b. ROS1 overexpression;
   c. ROS1 mutation(s)
      i. e.g., G2032R, D2033N, L2155S;
   d. chromosomal rearrangements involving the ROS1 gene;
21. SOS1 alterations
   a. SOS1 amplification;
   b. SOSi overexpression;
   c. SOS1 mutation(s);
22. RAC1 alterations
   a. RAC1 amplification;
   b. RAC1 overexpression;
   c. RAC1 mutation(s);
23. MDM2 alterations
   a. MDM2 amplification
   b. MDM2 overexpression
   c. MDM2 amplification in combination with functional p53
   d. MDM2 amplification in combination with wild-type p53
24. RAS wild-type
   a. KRAS wild-type
   a. HRAS wild-type
   b. NRAS wild-type

In some embodiments, the cancer to be treated with an SOS1 inhibitor of the present disclosure is selected from the group consisting of:
(a) lung adenocarcinoma harboring a KRAS mutation selected from the group consisting of G12C, G12V, G12D and G12R;
(b) colorectal adenocarcinoma harboring a KRAS mutation selected from the group consisting of G12D, G12V, G12C, G12R and G13D; and
(c) pancreatic adenocarcinoma harboring a KRAS mutation selected from the group consisting of G12D, G12V, G12R, G12C and Q61 H.

In some embodiments, the disease/condition to be treated/prevented with the SOS1 inhibitor compound of the present disclosure is a RASopathy selected from the group consisting of Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

### EXAMPLES

### Materials and Methods

Solvents, reagents and starting materials were purchased from commercial vendors and used as received unless otherwise described. All reactions were performed at room temperature unless otherwise stated. Compound identity and purity confirmations were performed by LCMS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in Table 1 below. Retention times RT are reported in minutes.

TABLE 1
LCMS solvent gradients for compound analysis.

| | | |
|---|---|---|
| Time (min) | % A | % B |

Method 1 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Method 2 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

NMR was also used to characterize final compounds. NMR spectra were obtained on a Bruker AVIII 400 Nanobay with 5 mm BBFO probe. Optionally, compound R_{f} values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector. Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in Table 2 below.

TABLE 1
LCMS solvent gradients for compound purification.

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

### Example 1. Synthesis of Intermediates

### Synthesis of 4-chloro-7-methoxy-1-methyl-pyrido[3,4-d]pyridazine

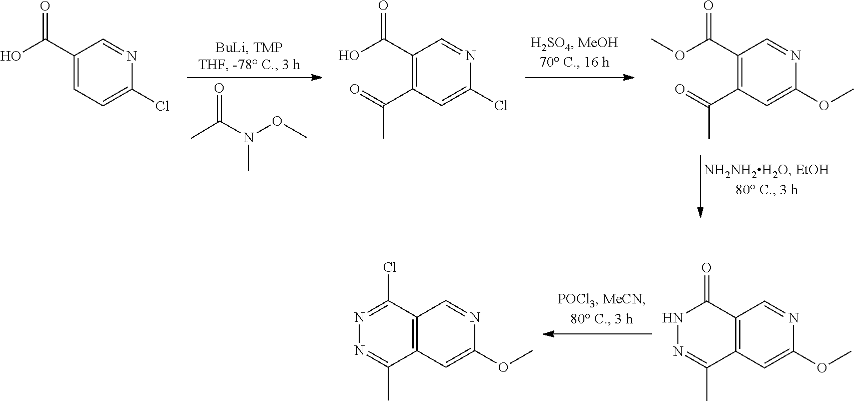

Step 1: To a stirred solution of 2,2,6,6-tetramethylpiperidine (6.43 mL, 38.08 mmol) in dry THF (20.1 mL), n-butyllithium solution in hexane (2.5M, 17.26 mL, 43.16 mmol) was added dropwise at −78° C. under nitrogen atmosphere. The reaction mixture was stirred for 30 minutes. 6-Chloronicotinic acid (2000 mg, 12.69 mmol) dissolved in dry THF (20.1 mL) was added dropwise to the above reaction mixture at −78° C. The obtained mixture was stirred for 1 hour at −78° C. Then N-methoxy-N-methylacetamide (5.4 mL, 50.78 mmol) was added dropwise and the reaction mixture was stirred for 1.5 h at −78° C. The reaction mixture was quenched at −78° C. with 1N HCl solution. After warming up to room temperature, the two layers were separated, the aqueous layer was extracted with ethyl acetate, and the organic layers were combined, dried over anhydrous Na₂SO₄, and then concentrated in vacuo. The solid precipitated out was collected by vacuum filtration washing with dichloromethane to afford 4-acetyl-6-chloro-pyridine-3-carboxylic acid (801 mg, 4.01 mmol, 31.6% yield) as a white solid. UPLC-MS (ES⁺, Short acidic): 1.14 min, m/z 200.0 [M+H]⁺ (95%)

Step 2: To a stirring solution of 4-acetyl-6-chloro-pyridine-3-carboxylic acid (1266.mg, 6.34 mmol) in methanol (15 mL) was added sulfuric acid (1.05 mL, 19.67 mmol). The mixture was stirred at 70° C. overnight. The reaction mixture was concentrated, and the residue taken up in dichloromethane and a sat. aq. Na₂CO₃ solution. The organic phase was extracted 3×, dried over MgSO₄ and concentrated. The crude residue was purified by flash column chromatography (12 g, eluent methanol in dichloromethane 0-2%) to afford methyl 4-acetyl-6-methoxy-pyridine-3-carboxylate (647 mg, 3.09 mmol, 48.7% yield) as a yellow solid. UPLC-MS (ES⁺, Short acidic): 1.41 min, m/z 210.1 [M+H]⁺ (100%). ¹H NMR (400 MHz, CDCl₃): δ 8.79-8.78 (m, 1H), 6.58-6.57 (m, 1H), 4.01 (s, 3H), 3.89 (s, 3H), 2.50 (s, 3H)

Step 3: Methyl 4-acetyl-6-methoxy-pyridine-3-carboxylate (1300 mg, 6.21 mmol) and hydrazine hydrate (362.78 μL, 7.46 mmol) were mixed in ethanol (6 mL). The reaction mixture was heated at 80° C. for two hours. The reaction mixture was concentrated in vacuo. The solid was filtered, washing with tert-butyl methyl ether and dried to afford 7-methoxy-1-methyl-3H-pyrido[3,4-d]pyridazin-4-one (370 mg, 1.93 mmol, 31.1% yield) as a white solid. UPLC-MS (ES+, Short acidic): 1.14 min, m/z 192.1 [M+H]⁺ (89%)

Step 4: To a solution of 7-chloro-1-methyl-3H-pyrido[3,4-d]pyridazin-4-one (370.mg, 1.94 mmol) in acetonitrile (6.102 mL) was added phosphorus oxychloride (631.34 uL, 6.77 mmol). The reaction mixture was heated at 80° C. for 2 hours. The reaction mixture was cooled down and poured over ice then basified with sat. aq. Na₂CO₃. Ethyl acetate was added, and the two phases were separated. The aqueous phase was re-extracted with ethyl acetate. The combined organic extracts were passed through phases separating filter paper and was concentrated in vacuo to afford 4-chloro-7-methoxy-1-methyl-pyrido[3,4-d]pyridazine (227 mg, 1.0829 mmol, 55.96% yield) as an orange solid. The material was telescoped through to the next step without further purification. UPLC-MS (ES+, Short acidic): 1.33 min, m/z 210.0 [M+H]⁺ (18%, 48%—is the Cl replaced by OMe in the LCMS sample)

### Synthesis of 3-bromo-5-chloro-8-methyl-pyrido[2,3-d]pyridazine

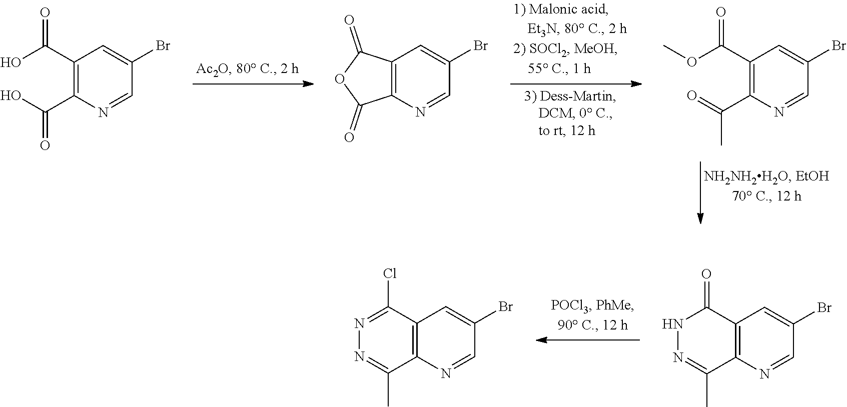

Step 1: A mixture of 5-bromopyridine-2,3-dicarboxylic acid (2 g, 8.13 mmol) and acetic anhydride (4 mL, 42.32 mmol) was stirred at 80° C. for 2 hours. The mixture was concentrated in vacuo and the residual solid was triturated with petroleum ether to afford 3-bromofuro[3,4-b]pyridine-5,7-dione (1.741 g, 7.636 mmol, 93.9% yield) as an off-white solid. UPLC-MS (ES+, short acidic): 1.14 min, not ionizable (98%); ¹H-NMR (400 MHz, CDCl₃): δ 9.21 (d, J=1.6 Hz, 1H), 8.49 (d, J=1.6 Hz, 1H)

Step 2: A mixture of 3-bromofuro[3,4-b]pyridine-5,7-dione, malonic acid (900.mg, 8.65 mmol), triethylamine (1.5 mL, 10.79 mmol) was stirred for 2 hours at 80° C. in a 20 mL sealed flask. HCl in methanol was added until pH 3-4. The reaction was transferred in a round bottom flask and the solvent removed under vacuo. The crude was dissolved in methanol (8 mL), cooled to 0° C. and thionyl chloride (1.05 mL, 14.39 mmol) was added dropwise. The reaction was heated to 55° C. for 1 hour. The solvent was evaporated. Water was added followed by extraction with dichloromethane (3×). The combined organic phases were washed with brine, dried over a phase separator and the solvent removed in vacuo. The crude was dissolved in dichloromethane (16 mL), cooled to 0° C. and Dess-Martin periodinane (3357.98 mg, 7.92 mmol) was added. The reaction was stirred at room temperature overnight. More Dess-Martin periodinane (3357.98 mg, 7.92 mmol) added and the reaction mixture was stirred overnight. Water was added and dichloromethane was evaporated. The aqueous phase was extracted with ethyl acetate (2×). The combined organic phases were washed with water and brine, dried over Na₂SO₄ and the solvent removed under reduce pressure. The crude was purified by column chromatography (eluent ethyl acetate in petroleum ether 0-30%) to give methyl 2-acetyl-5-bromo-pyridine-3-carboxylate (326 mg, 1.2632 mmol, 17.5% yield). UPLC-MS (ES+, short acidic): 1.60 min, m/z 257.9/259.9 [M+H]⁺ (93%); ¹HNMR (400 MHz, CDCl₃): δ 8.76 (d, J=2.4 Hz, 1H), 8.10 (d, J=2.4 Hz, 1H), 3.93 (s, 3H), 2.67 (s, 3H)

Step 3: Methyl 2-acetyl-5-bromo-pyridine-3-carboxylate (0.29 mL, 1.26 mmol) and hydrazine hydrate (0.09 mL, 1.89 mmol) were mixed in ethanol (3.5 mL). The reaction mixture was heated at 70° C. overnight. It was then concentrated to dryness and triturated with tert-butyl methyl ether then filtered, washed with tert-butyl methyl ether to give 3-bromo-8-methyl-6H-pyrido[2,3-d]pyridazin-5-one (266 mg, 1.1081 mmol, 87.7% yield) as a white solid. UPLC-MS (ES+, short acidic): 1.26 min, m/z 240.0/242.0 [M+H]⁺ (100%); ¹H-NMR (400 MHz, CDCl₃): δ 9.92 (br s, 1H), 9.11 (d, J=2.4 Hz, 1H), 8.82 (d, J=2.4 Hz, 1H), 2.65 (s, 3H)

Step 4: 3-bromo-8-methyl-6H-pyrido[2,3-d]pyridazin-5-one (123.mg, 0.5100 mmol) was dissolved in toluene (3 mL) followed by the addition of phosphorus oxychloride (0.17 mL, 1.79 mmol). The reaction was heated to 90° C. overnight in a sealed vial. The mixture was cooled down, the solvent was evaporated and the residue was poured in ice/sat. sol. Na₂CO₃. The product was extracted ethyl acetate (3×); the combined organic phases were washed with brine and dried over Na₂SO₄ before concentration to afford 3-bromo-5-chloro-8-methyl-pyrido[2,3-d]pyridazine (109 mg, 0.4217 mmol, 82.296% yield) as an orange solid. UPLC-MS (ES+, short acidic): 1.45 min, m/z 258.0/259.9/261.9 [M+H]⁺ (86%); ¹HNMR (400 MHz, CDCl₃): δ 9.27 (d, J=2.4 Hz, 1H), 8.71 (d, J=2.4 Hz, 1H), 3.08 (s, 3H)

### Synthesis of 5-chloro-1,8-dimethyl-pyrido[2,3-d]pyridazin-2-one

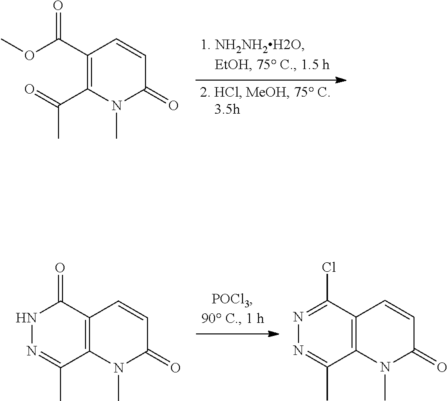

Step 1: To a stirring solution of methyl 2-acetyl-1-methyl-6-oxo-pyridine-3-carboxylate (3.73 g, 17.83 mmol) in ethanol (15 mL) was added hydrazine hydrate (2.17 mL, 44.6 mmol). The mixture was heated to 75° C. for 1.5 h. Hydrogen chloride (3 mL, 36.0 mmol) was added and the reaction was stirred at 75° C. for 3.5 h. The reaction was cooled down with an ice bath, filtered and washed with cold ethanol, then dried in the vacuum oven to afford 1,8-dimethyl-6H-pyrido[2,3-d]pyridazine-2,5-dione (3.41 g, 17.8 mmol, quantitative yield) as a white solid.

UPLC-MS (ES+, Short acidic): 0.94 min, m/z 192.0 [M+H]⁺ (100%)

¹H NMR (400 MHz, DMSO-d₆): δ 12.89 (s, 1H), 8.03 (d, J=9.5 Hz, 1H), 6.79 (d, J=9.4 Hz, 1H), 3.76 (s, 3H), 2.69 (s, 3H)

Step 2: 1,8-dimethyl-6H-pyrido[2,3-d]pyridazine-2,5-dione (2 g, 10.46 mmol) in phosphorus oxychloride (6 mL, 64.37 mmol) was heated to 90° C. in a sealed vial for lh. The reaction was concentrated to dryness. The residue was purified by column chromatography using as eluent a gradient 0-20% MeOH in DCM to afford 5-chloro-1,8-dimethyl-pyrido[2,3-d]pyridazin-2-one (1.12 g, 5.3427 mmol, 51.1% yield) as an orange solid.

UPLC-MS (ES+, Short acidic): 1.30 min, m/z 210.0/212.0 [M+H]⁺ (100%) [00139]¹H NMR (400 MHz, DMSO-d₆): δ 8.11 (d, J=9.7 Hz, 1H), 7.09 (d, J=9.7 Hz, 1H), 3.82 (s, 3H), 3.05 (s, 3H)

### Synthesis of 3-bromo-5-chloro-1,8-dimethyl-pyrido[2,3-d]pyridazin-2-one

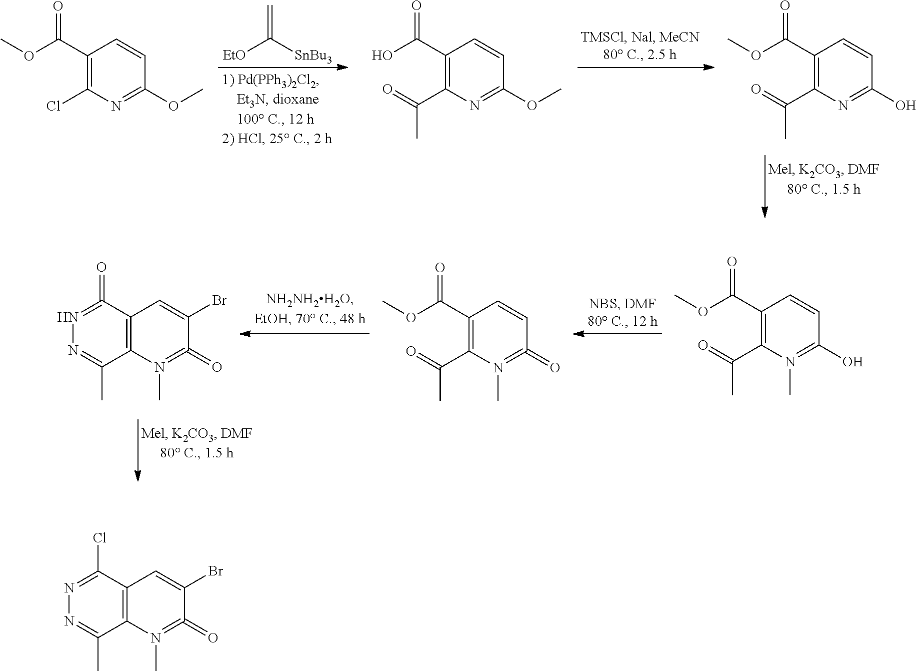

Step 1: A solution of methyl 2-chloro-6-methoxynicotinate (297.62 μL, 4.96 mmol), tributyl(1-ethoxyvinyl)tin (2.01 mL, 5.95 mmol), triethylamine (1.73 mL, 12.4 mmol) in 1,4-dioxane (5 mL) in a vial was degassed with N₂ for 10 minutes. Bis(triphenylphosphine)palladium(II) dichloride (522.22 mg, 0.7400 mmol) was added and the mixture was degassed for a further 5 min. The vial was sealed and the reaction was heated to 100° C. overnight. The reaction was cooled down and 2N HCl in water (6. mL, 12 mmol) was added. The reaction was stirred at room temperature for 2 hours. The mixture was concentrated and taken up in water and dichloromethane. The aqueous phase was extracted with dichloromethane (3×). The combined organic extracts were dried over MgSO₄ and concentrated in vacuo. The crude residue was purified by flash column chromatography (25 g, eluent ethyl acetate in petroleum ether 0-15%) to afford methyl 2-acetyl-6-methoxy-pyridine-3-carboxylate (1.06 g, 5.0669 mmol, 100% yield) as a yellow oil.

UPLC-MS (ES⁺, Short acidic): 1.65 min, m/z 210.1 [M+H]⁺ (100%)

¹H NMR (400 MHz, DMSO-d₆): δ 7.99 (d, J=8.6 Hz, 1H), 6.83 (d, J=8.6 Hz, 1H), 3.99 (s, 3H), 3.87 (s, 3H), 2.61 (s, 3H)

Step 2: Methyl 2-acetyl-6-methoxy-pyridine-3-carboxylate (300.mg, 1.43 mmol) was dissolved in acetonitrile (2 mL) followed by the addition of sodium iodide (429.89 mg, 2.87 mmol) and chlorotrimethylsilane, redistilled (371.43 μL, 2.87 mmol). The vial was sealed and heated to 80° C. for 2.5 hours. Water was added and the mixture was extracted with dichloromethane (3×). The organic layer was passed through a phase separator and the solvent removed under reduced pressure. The residue was then purified by flash column chromatography (12 g, eluent methanol in dichloromethane 0-5%) to give methyl 2-acetyl-6-hydroxy-pyridine-3-carboxylate (66 mg, 0.3382 mmol, 23.6% yield) as a purple solid.

UPLC-MS (ES⁺, Short acidic): 1.06 min, m/z 196.1 [M+H]⁺ (100%)

¹H NMR (400 MHz, CDCl₃): δ 7.92 (d, J=9.6 Hz, 1H), 6.58 (d, J=9.6 Hz, 1H), 3.86 (s, 3H), 2.61 (s, 3H)

Step 3: To a stirring solution of methyl 2-acetyl-6-hydroxy-pyridine-3-carboxylate (350.mg, 1.79 mmol) and potassium carbonate (743.56 mg, 5.38 mmol) in DMF (3 mL) was added iodomethane (446.57 μL, 7.17 mmol) in a sealed vial. The reaction was heated to 80° C. for 1.5 hours. The reaction was partitioned between dichloromethane and water. The aqueous layer was extracted with dichloromethane. The organic phase was washed with water (2×), brine, passed through a phase separator and concentrated under reduced pressure. The residue was then purified by flash column chromatography (25 g eluent methanol in dichloromethane 0-5%) to afford methyl 2-acetyl-1-methyl-6-oxo-pyridine-3-carboxylate (263 mg, 1.2572 mmol, 70.1% yield) as a white solid.

UPLC-MS (ES⁺, Short acidic): 1.31 min, m/z 210.1 [M+H]⁺ (100%)

¹H NMR (400 MHz, CDCl₃): δ 7.83 (d, J=9.7 Hz, 1H), 6.54 (d, J=9.7 Hz, 1H), 3.84 (s, 3H), 3.43 (s, 3H), 2.60 (s, 3H)

Step 4: To a stirring solution of methyl 2-acetyl-1-methyl-6-oxo-pyridine-3-carboxylate (229.mg, 1.09 mmol) in DMF (1.5 mL) was added N-bromosuccinimide (233.79 mg, 1.31 mmol). The mixture was stirred at 80° C. overnight in a sealed vial. The reaction was partitioned between dichloromethane and water. The aqueous layer was extracted with dichloromethane (2×). The organic phase was washed with water (3×), passed through a phase separator and concentrated under reduced pressure. The residue was then purified by flash column chromatography (12 g, eluent ethyl acetate in petroleum ether 0-80%) to afford methyl 2-acetyl-5-bromo-1-methyl-6-oxo-pyridine-3-carboxylate (290 mg, 1.0066 mmol, 91.9% yield) as a colourless oil.

UPLC-MS (ES⁺, Short acidic): 1.54 min, m/z 287.9/289.9 [M+H]⁺ (100%)

¹H NMR (400 MHz, CDCl₃): δ 8.24 (s, 1H), 3.85 (s, 3H), 3.50 (s, 3H), 2.59 (s, 3H)

Step 5: To a stirring solution of methyl 2-acetyl-5-bromo-1-methyl-6-oxo-pyridine-3-carboxylate (50.mg, 0.1700 mmol) in ethanol (1 mL) was added hydrazine hydrate (12.67 μL, 0.2600 mmol). The vial was sealed and heated to 70° C. for 5 hours. Further hydrazine hydrate (13 μL, 0.2700 mmol) was added and the reaction was stirred over the weekend. The reaction was concentrated to dryness, the residue was then purified by flash column chromatography (4 g, eluent methanol in dichloromethane 0-5%) to afford 3-bromo-1,8-dimethyl-6H-pyrido[2,3-d]pyridazine-2,5-dione (34 mg, 0.1259 mmol, 72.5% yield) as apale yellow solid.

UPLC-MS (ES⁺, Short acidic): 1.11 min, m/z 270/272 [M+H]⁺ (81%)

¹H NMR (400 MHz, DMSO-d₆): δ 13.0 (s, 1H), 8.40 (s, 1H), 3.82 (s, 3H), 2.69 (s, 3H)

Step 6: 3-bromo-1,8-dimethyl-6H-pyrido[2,3-d]pyridazine-2,5-dione (760.mg, 2.81 mmol) in phosphorus oxychloride (4. mL, 42.91 mmol) was heated to 80° C. in a sealed vial for 2 hours. The reaction was concentrated to dryness and the residue was partitioned between dichloromethane and water. The aqueous layer was extracted with dichloromethane (7×). The organic phase was washed with brine, passed through a phase separator and concentrated under reduced pressure. The residue was then purified by flash column chromatography (12 g, eluent methanol in dichloromethane 0-20%) to afford 3-bromo-5-chloro-1,8-dimethyl-pyrido[2,3-d]pyridazin-2-one (1.04 g, 3.6045 mmol, 128.0% yield) as an orange solid.

UPLC-MS (ES⁺, Short acidic): 1.40 min, m/z 287.9/289.9/291.9 [M+H]⁺ (86%)

¹H NMR (400 MHz, DMSO-d₆): δ 8.53 (s, 1H), 3.89 (s, 3H), 3.06 (s, 3H)

### Synthesis of 8-chloro-2-methoxy-5-methyl-pyrido[2,3-d]pyridazine

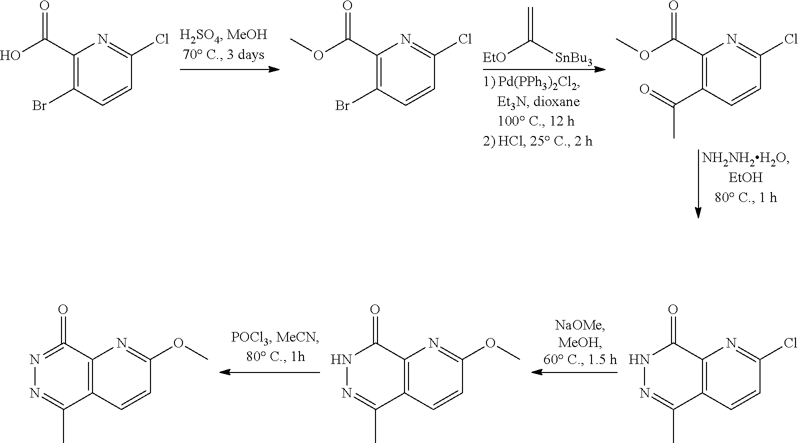

Step 1: To a stirring solution of 3-bromo-6-chloropyridine-2-carboxylic acid (3.00 g, 12.7 mmol) in methanol (25.4 mL) was added sulfuric acid (2.03 mL, 38.1 mmol). The mixture was stirred at 70° C. for 3 days. The reaction mixture was concentrated, and the residue taken up in DCM and a sat. aq. Na₂CO₃ solution. The organic phase was extracted with DCM (×3), dried over Na₂SO₄ and concentrated. The crude residue was purified by column chromatography (40 g, 0-50% EtOAc in petroleum ether) to afford methyl 3-bromo-6-chloro-pyridine-2-carboxylate (2.94 g, 11.7 mmol, 92.6% yield) as a white solid.

UPLC-MS (ES+, short acidic): 1.66 min, m/z 249.9/251.8 [M+H]⁺ (100%)

¹H NMR (400 MHz, DMSO-d₆) δ 8.32 (d, J=8.5 Hz, 1H), 7.70 (d, J=8.5 Hz, 1H), 3.93 (s, 3H)

Step 2: A solution of methyl 3-bromo-6-chloro-pyridine-2-carboxylate (841 mg, 3.36 mmol), triethylamine (1.17 mL, 8.39 mmol), tributyl(1-ethoxyvinyl)tin (1.36 mL, 4.03 mmol) in 1,4-dioxane (11 mL) was degassed with N₂ for 5 min in a vial. Bis(triphenylphosphine)palladium(II) dichloride (236 mg, 0.34 mmol) was added and the mixture was degassed for a further 5 min. The vial was sealed, and the reaction was heated at 100° C. overnight. The reaction mixture was cooled down and HCl (2M in water) (8.4 mL, 16.8 mmol) was added and the obtained mixture stirred at rt for 1 h. The solution was partitioned between water and EtOAc. The aqueous layer was extracted with EtOAc (×3), the organic layers were combined, dried over Na₂SO₄, filtered, and concentrated under reduced pressure. The crude was purified by column chromatography using as eluent a gradient 0-100% EtOAc in petroleum ether to give methyl 3-acetyl-6-chloro-pyridine-2-carboxylate (455.2 mg, 2.1309 mmol, 63.5% yield) as a light yellow solid. 30% impurity of the diacetylated product.

UPLC-MS (ES+, Short acidic): 1.31 min, m/z 214.0 [M+H]⁺ (72%)

¹H NMR (400 MHz, DMSO-d₆) δ 8.39 (d, J=8.3 Hz, 1H), 7.89 (d, J=8.3 Hz, 1H), 3.86 (s, 3H), 2.59 (s, 3H)

Step 3: Methyl 3-acetyl-6-chloro-pyridine-2-carboxylate (420 mg, 1.97 mmol) and hydrazine hydrate (115 μL, 2.36 mmol) were mixed in ethanol (2 mL). The reaction mixture was heated at 80° C. for 1 h. The reaction mixture was concentrated under reduced pressure to afford 2-chloro-5-methyl-7H-pyrido[2,3-d]pyridazine-8-one (384 mg, 1.96 mmol, 99.8% yield) as an orange solid.

UPLC-MS (ES+, Short acidic): 0.97 min, m/z 218.0 [M+Na]⁺ (91%)

¹H NMR (400 MHz, DMSO-d₆) δ 12.83 (s, 1H), 8.44 (d, J=8.6 Hz, 1H), 8.04 (d, J=8.6 Hz, 1H), 2.52 (s, 3H).

Step 4: To a solution of 2-chloro-5-methyl-7H-pyrido[2,3-d]pyridazin-8-one (177 mg, 0.90 mmol) in methanol (3 mL) at rt was added sodium methoxide (0.23 mL, 1.81 mmol). The reaction mixture was heated at 60° C. for 1.5 h. The reaction mixture was concentrated under reduced pressure, and purified by column chromatography using as eluent a gradient 0-20% MeOH in DCM to afford 2-methoxy-5-methyl-7H-pyrido[2,3-d]pyridazin-8-one (115 mg, 0.60 mmol, 66.2% yield) as a white solid.

UPLC-MS (ES+, Short acidic): 1.11 min, m/z 192.0 [M+H]⁺ (100%) [00171]¹H NMR (400 MHz, DMSO-d₆) δ 12.62 (s, 1H), 8.26 (d, J=8.8 Hz, 1H), 7.36 (d, J=8.9 Hz, 1H), 4.03 (s, 3H), 2.48 (s, 3H)

Step 5: To a solution of 2-methoxy-5-methyl-7H-pyrido[2,3-d]pyridazin-8-one (115 mg, 0.60 mmol) in MeCN (3 mL) was added phosphorus oxychloride (0.19 mL, 2.09 mmol). The reaction mixture was heated to 80° C. for 1 h. The reaction was cooled to rt, poured into ice and then basified with sat. aq. NaHCO₃. DCM was added and the two phases separated. The aqueous phase was extracted with DCM (×3), and organic phases were combined, dried over Na₂SO₄, filtered and concentrated under reduced pressure to afford 8-chloro-2-methoxy-5-methyl-pyrido[2,3-d]pyridazine (125 mg, 0.60 mmol, 100% yield) as a light brown solid. The product was used in the next step without further purification.

UPLC-MS (ES+, Short acidic): 1.44 min, m/z 210.0/212.0 [M+H]⁺ (92%)

¹H NMR (400 MHz, DMSO-d₆) δ 8.59 (dd, J=0.7, 9.0 Hz, 1H), 7.57 (dd, J=0.8, 9.1 Hz, 1H), 4.13 (d, J=0.8 Hz, 3H), 2.89 (d, J=0.7 Hz, 3H)

### Example 2. Synthesis of 6-cyclopentyl-1-methyl-4-[1-[3-(trifluoromethyl)-phenyl]ethylamino]pyrido[3,4-d]pyridazin-7-one (1)

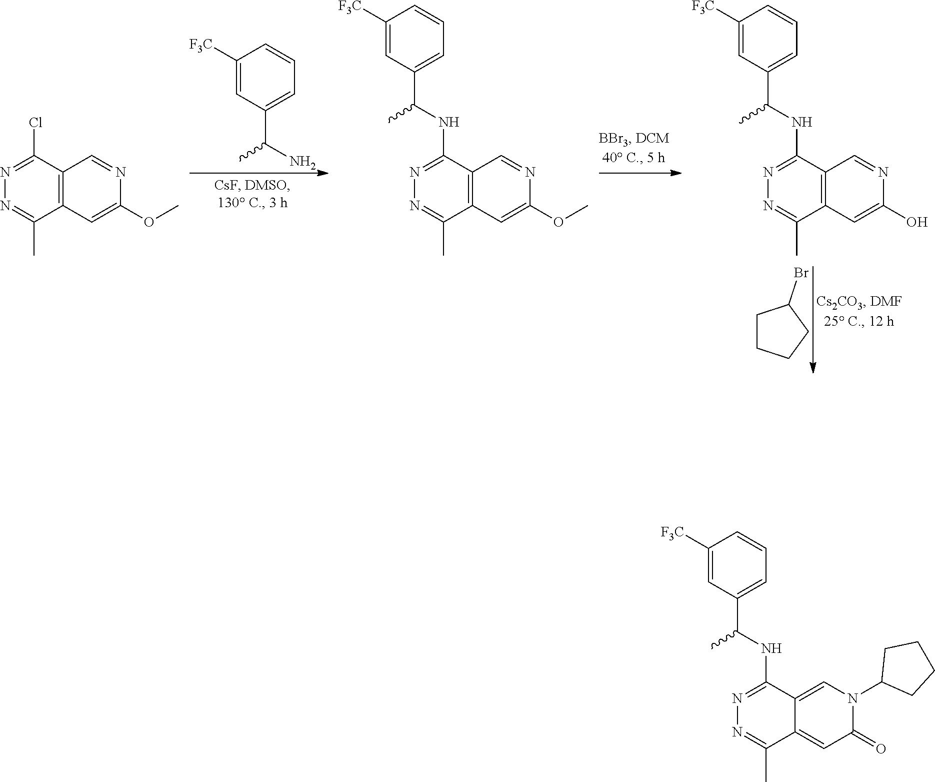

Step 1: To a vial was added 4-chloro-7-methoxy-1-methyl-pyrido[3,4-d]pyridazine (200.mg, 0.9500 mmol) and alpha-methyl-3-(trifluoromethyl)benzylamine (150.41 uL, 0.9500 mmol) in DMSO (2.0018 mL). Cesium fluoride (217.38 mg, 1.43 mmol) was added and the vial was sealed. The reaction mixture was heated at 130° C. for 3 hours. The reaction was cooled to room temperature and partitioned between water and ethyl acetate. The two phases were separated and the aqueous was re-extracted with ethyl acetate. The combined organic extracts were passed through phases separating filter paper and concentrated in vacuo. The crude material was purified by flash column chromatography (4 g, eluent methanol in dichloromethane 0-20%) like fractions were pooled and concentrated in vacuo to afford 7-methoxy-1-methyl-N-[1-[3-(trifluoromethyl)phenyl]ethyl]pyrido[3,4-d]pyridazin-4-amine (200 mg, 0.5520 mmol, 57.8% yield) as a brown oil. UPLC-MS (ES+, Short acidic): 1.50 min, m/z 363.5 [M+H]⁺ (91%); ¹H NMR (400 MHz, CDCl₃): δ 8.99 (s, 1H), 7.72-7.66 (m, 2H), 7.52-7.41 (m, 2H), 5.74-5.66 (m, 1H), 5.38 (d, J=6.5 Hz, 1H), 4.08 (s, 3H), 2.69 (s, 3H), 1.70 (d, J=6.9 Hz, 3H)

Step 2: To a solution of 7-methoxy-1-methyl-N-[1-[3-(trifluoromethyl)phenyl]ethyl]pyrido[3,4-d]pyridazin-4-amine (160.mg, 0.4400 mmol) in DCM (2.0072 mL) at 0° C. was added boron tribromide (1M, 0.88 mL, 0.8800 mmol) dropwise. The reaction mixture was warmed to room temperature then heated at 40° C. for 5 hours. The reaction mixture was added slowly to ice cold NaHCO3 solution (solution was pH 7 at the end of the addition). The two phases were separated, and the mixture was extracted with ethyl acetate (2×). The combined ethyl acetate extracts were passed through phase separating filter paper and concentrated in vacuo to afford 1-methyl-4-[1-[3-(trifluoromethyl)phenyl]ethylamino]pyrido[3,4-d]pyridazin-7-ol (59 mg, 0.1694 mmol, 38.3% yield) as a yellow oil. The material was used in the next step without further purification. UPLC-MS (ES+, Short acidic): 1.34 min, m/z 349.2 [M+H]⁺ (100%)

Step 3: To 1-methyl-4-[1-[3-(trifluoromethyl)phenyl]ethylamino]-6H-pyrido[3,4-d]pyridazin-7-one (59.mg, 0.1700 mmol) in DMF (1.1244 mL) was added bromocyclopentane (0.02 mL, 0.22 mmol) and cesium carbonate (82.79 mg, 0.25 mmol). The reaction mixture was stirred at room temperature overnight. The reaction mixture was concentrated in vacuo. The crude material was purified by flash column chromatography (4 g, eluent ethyl acetate in petroleum ether 0-100% then methanol in dichloromethane 0-20%) like fractions were pooled and concentrated in vacuo to afford 6-cyclopentyl-1-methyl-4-[1-[3-(trifluoromethyl)phenyl]ethylamino]pyrido[3,4-d]pyridazin-7-one (5.6 mg, 0.0134 mmol, 7.9% yield) as a brown solid. UPLC-MS (ES⁺, long acidic): 3.03 min, m/z 417.5 [M+H]⁺ (90%); UPLC-MS (ES+, Short acidic): 1.57 min, m/z [M+H]⁺ (80%); ¹H NMR (400 MHz, DMSO-d₆): δ 9.04 (s, 1H), 7.77-7.69 (m, 3H), 7.61-7.53 (m, 2H), 6.54 (s, 1H), 5.55-5.46 (m, 1H), 5.27-5.17 (m, 1H), 2.36 (s, 3H), 2.18-2.08 (m, 2H), 1.97-1.89 (m, 4H), 1.79-1.71 (m, 2H), 1.59 (d, J=7.1 Hz, 3H)

The following examples were prepared in a similar manner, starting from the corresponding amine and halide reagents.

| Compound | Structure | Analytical data |
|---|---|---|
| 2 | 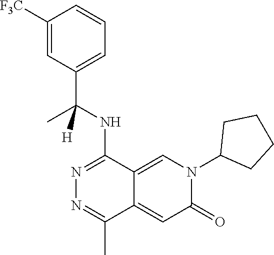 [SMILES: Cc1nnc(N[C@H](C)c2cccc(C(F)(F)F)c2)c2cn(C3CCCC3)c(=O)cc12] | UPLC-MS (ES⁺, short acidic): 1.60 min, m/z 417.6 [M + H]⁺ (93%) UPLC-MS (ES⁺, short acidic): 3.14 min, m/z 417.7 [M + H]⁺ (100%) ¹H NMR (400MHz, DMSO-d₆): δ 9.00 (s, 1H), 7.76-7.71 (m, 2H), 7.62-7.54 (m, 3H), 6.53 (d, J = 0.4Hz, 1H), 5.55 (q, J = 7.2Hz, 1H), 5.27-5.17 (m, 1H), 2.37 (s, 3H), 2.19- 2.10 (m, 2H), 2.00-1.88 (m, 4H), 1.78-1.72 (m, 2H), 1.60 (d, J = 7.2 Hz, 3H) |
| 3 | 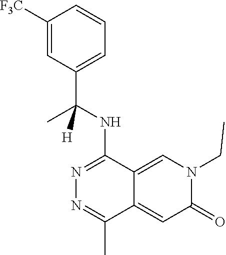 [SMILES: CCn1cc2c(N[C@H](C)c3cccc(C(F)(F)F)c3)nnc(C)c2cc1=O] | UPLC-MS (ES⁺, short acidic): 1.49 min, m/z 377.6 [M + H]⁺ (100%) UPLC-MS (ES⁺, final purity): 3.01 min, m/z 377.6 [M + H]⁺ (94%) ¹H NMR (400MHz, DMSO-d₆): δ 9.24 (s, 1H), 7.76-7.70 (m, 2H), 7.60-7.52 (m, 3H), 6.53 (s, 1H), 5.46 (aqq quint, J = 6.8Hz, 1H), 4.21-4.11 (m, 2H), 2.36 (s, 3H), 1.55 (d, J = 7.2Hz, 3H), 1.38 (t, J = 7.2Hz, 3H) |
| 4 | 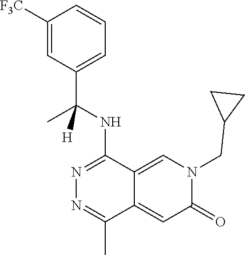 [SMILES: Cc1nnc(N[C@H](C)c2cccc(C(F)(F)F)c2)c2cn(CC3CC3)c(=O)cc12] | UPLC-MS (ES⁺, short acidic): 1.49 min, m/z 403.2 [M + H]⁺ (95%) UPLC-MS (ES⁺, final purity): 3.33 min, m/z 403.5 [M + H]⁺ (95%) ¹H NMR (400MHz, DMSO-d₆): δ 9.22 (s, 1H), 7.76-7.69 (m, 2H), 7.61-7.52 (m, 3H), 6.55 (s, 1H), 5.49 (app quint, J = 6.8Hz, 1H), 4.11-3.94 (m, 2H), 2.37 (s, 3H), 1.57 (d, J = 6.8Hz, 3H), 1.44-1.33 (m, 1H), 0.59-0.46 (m, 4H) |
| 5 | 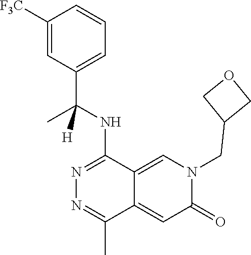 [SMILES: Cc1nnc(N[C@H](C)c2cccc(C(F)(F)F)c2)c2cn(CC3COC3)c(=O)cc12] | UPLC-MS (ES⁺, Long acidic): 3.29 min, m/z 419.5 [M + H]⁺ (91%). ¹H-NMR (400MHz, DMSO-d₆): δ 9.29 (s, 1H), 7.75 (s, 1H), 7.73-7.69 (m, 1H), 7.62-7.51 (m, 3H), 6.54 (s, 1H), 5.51-5.44 (m, 1H), 4.70-4.64 (m, 2H), 4.51-4.46 (m, 2H), 4.43 (d, J = 7.6Hz, 2H), 3.52-3.44 (m, 1H), 2.35 (s, 3H), 1.56 (d, J = 6.8Hz, 3H). |
| 6 | 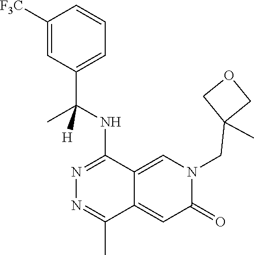 [SMILES: Cc1nnc(N[C@H](C)c2cccc(C(F)(F)F)c2)c2cn(CC3(C)COC3)c(=O)cc12] | UPLC-MS (ES⁺, Long acidic): 3.32 min, m/z 433.5 [M + H]⁺ (100%). ¹H-NMR (400MHz, DMSO-d₆): δ 9.22 (s, 1H), 7.74 (s, 1H), 7.72-7.69 (m, 1H), 7.60-7.54 (m, 3H), 6.58 (m, 1H), 5.53-5.45 (m, 1H), 4.66 (t, J = 5.6Hz, 2H), 4.33 (d, J = 0.4Hz, 2H), 4.23 (dd, J = 6.0, 2.4Hz, 2H), 2.37 (s, 3H), 1.56 (d, J = 7.2Hz, 3H), 1.27 (s, 3H). |
| 7 | 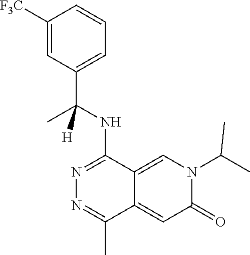 [SMILES: Cc1nnc(N[C@H](C)c2cccc(C(F)(F)F)c2)c2cn(C(C)C)c(=O)cc12] | UPLC-MS (ES⁺, Long acidic): 2.78 min, m/z 391.4 [M + H]⁺ (95%). ¹H-NMR (400MHz, DMSO-d₆): δ 9.08 (s, 1H), 7.76-7.70 (m, 3H), 7.60-7.54 (m, 2H), 6.54 (s, 1H), 5.55-5.46 (m, 1H), 5.34-5.24 (m, 1H), 2.36 (s, 3H), 1.59 (d, J = 6.8Hz, 3H), 1.47 (d, J = 6.8Hz, 6H). |
| 8 | 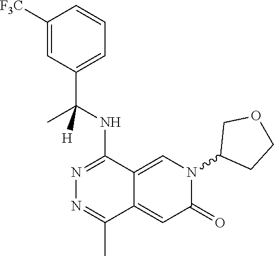 [SMILES: Cc1nnc(N[C@H](C)c2cccc(C(F)(F)F)c2)c2cn(C3CCOC3)c(=O)cc12] | UPLC-MS (ES⁺, Long acidic): 2.66 min, m/z 419.5 [M + H]⁺ & 2.68 min, m/z 419.4 [M + H]⁺ (90%) ¹H-NMR (400MHz, CD₃OD): δ 8.95 (s, 1H), 7.76-7.70 (m, 2H), 7.44-7.47 (m, 3H), 6.76 (s, 1H), 5.75-5.64 (m, 1H), 5.55-5.43 (m, 1H), 4.33-4.23 (m, 1H), 4.20-4.15 (m, 1H), 4.12-4.05 (m, 1H), 3.96-3.89 (m, 1H), 2.78-2.63 (m, 1H), 2.47 (s, 3H), 2.33-2.20 (m, 1H), 1.66 (d, J = 7.1Hz, 3H). |

### Example 3. Synthesis of 8-methyl-3-[(3S)-tetrahydrofuran-3-yl]oxy-N-[1-[3-(trifluoromethyl)phenyl]ethyl]pyrido[2,3-d]pyridazin-5-amine (9)

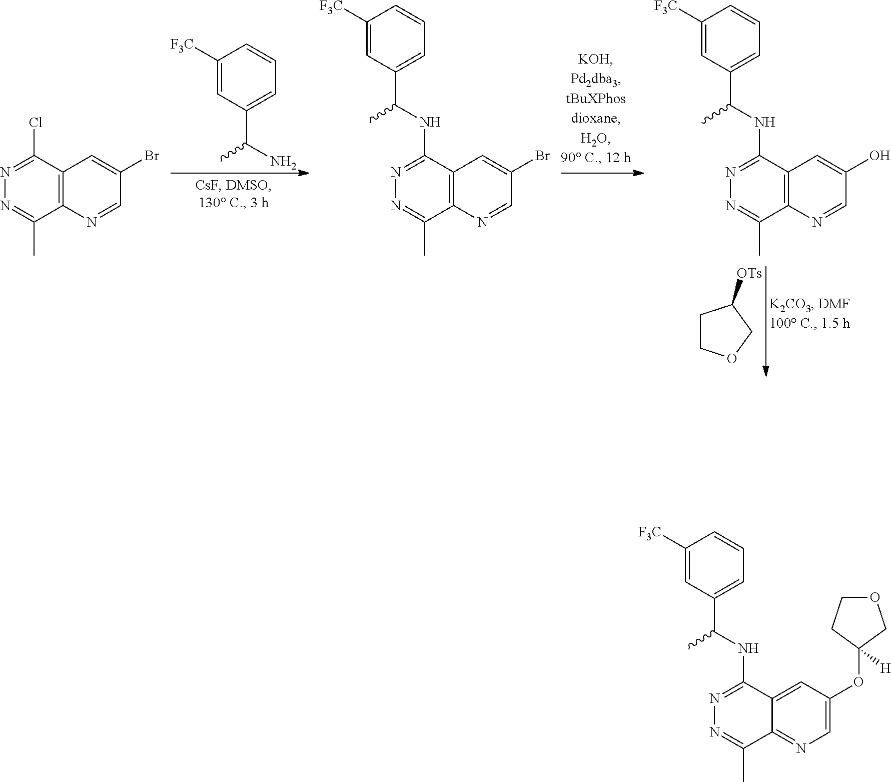

Step 1: A solution of 96 mg of a ˜1:1 mixture of 3-bromo-5-chloro-8-methyl-pyrido[2,3-d]pyridazine and 3,5-dichloro-8-methyl-pyrido[2,3-d]pyridazine, cesium fluoride (84.62 mg, 0.5600 mmol) and alpha-methyl-3-(trifluoromethyl)benzylamine (70.26 mg, 0.3700 mmol) in DMSO (1.0 mL) was heated to 130° C. overnight in a sealed vial. The reaction was diluted with ethyl acetate and washed with brine (2×). The organic layer was separated, dried over Na₂SO₄ and the solvent removed under reduce pressure. The crude was purified by flash column chromatography (eluent ethyl acetate in petroleum ether 0-100%) to give 93 mg of a mixture of 3-bromo-8-methyl-N-[1-[3-(trifluoromethyl)phenyl]ethyl]pyrido[2,3-d]pyridazin-5-amine (93 mg, 51% purity 0.1153 mmol, 23.6% yield), together with 3-chloro-8-methyl-N-[1-[3-(trifluoromethyl)phenyl]ethyl]pyrido[2,3-d]pyridazin-5-amine as a pale brown solid. UPLC-MS (ES⁺, Short acidic): 1.59 min, m/z 411.0/413.0 [M+H]⁺ (66%); ¹HNMR (400 MHz, CDCl₃): δ 9.11 (d, J=2.0 Hz, 1H), 8.41 (d, J=2.4 Hz, 1H), 7.67-7.61 (m, 2H), 7.47-7.43 (m, 1H), 7.39-7.34 (m, 1H), 5.66-5.59 (app quint, J=6.8 Hz, 1H), 2.85 (s, 3H), 1.66 (d, J=6.8 Hz, 3H)

Step 2: 66 mg of a mixture ˜1:1 of 3-bromo-8-methyl-N-[1-[3-(trifluoromethyl)phenyl]ethyl]pyrido[2,3-d]pyridazin-5-amine and 3-chloro-8-methyl-N-[1-[3-(trifluoromethyl)phenyl]ethyl]pyrido[2,3-d]pyridazin-5-amine and KOH (25.79 mg, 0.4600 mmol) were dissolved in 1,4-dioxane (0.4000 mL) and water (0.4000 mL). N₂ was bubbled through the reaction mixture for 5 minutes, followed by the addition of phosphine, bis(1,1-dimethylethyl)[2′,4′,6′-tris(1-methylethyl)[1,1′-biphenyl]-2-yl]- (13.01 mg, 0.0300 mmol) and tris(dibenzylideneacetone)dipalladium (0) (14.03 mg, 0.0200 mmol). The reaction mixture was heated at 90° C. for 1.5 hours. The reaction was filtered over celite and washed with ethyl acetate. The reaction was acidified with 1M aq. HCl and extracted with ethyl acetate (2×). The organic phases were combined, washed with brine, dried over Na₂SO₄ and the solvent removed under reduce pressure to give 8-methyl-5-[1-[3-(trifluoromethyl)phenyl]ethylamino]pyrido[2,3-d]pyridazin-3-ol (73 mg, 0.2096 mmol, 92.7% yield) as a yellow oil. The compound was carried to the next step without further purification. UPLC-MS (ES⁺, short acidic): 1.44 min, m/z 349.1 [M+H]⁺ (100%)

Step 3: 8-Methyl-5-[1-[3-(trifluoromethyl)phenyl]ethylamino]pyrido[2,3-d]pyridazin-3-ol (73.mg, 0.2100 mmol), potassium carbonate (28.97 mg, 0.2100 mmol) and [(3R)-tetrahydrofuran-3-yl]4-methylbenzenesulfonate (76.18 mg, 0.3100 mmol) were mixed in DMF (1.5 mL). The reaction mixture was heated at 100° C. for 3.5 hours. The reaction mixture was evaporated, and the crude was purified via prep HPLC (middle method), like fractions were pooled and concentrated in vacuo. The resulting product was passed through an SCX cartridge lg, eluting with NH₃ in methanol and concentrated to afford 8-methyl-3-[(3S)-tetrahydrofuran-3-yl]oxy-N-[1-[3-(trifluoromethyl)phenyl]ethyl]pyrido[2,3-d]pyridazin-5-amine (6 mg, 0.0143 mmol, 6.8416% yield) as a white solid (mixture of diastereomers). UPLC-MS (ES+, short acidic): 1.47 min, m/z 419.1 [M+H]⁺ (100%); UPLC-MS (ES⁺, Long acidic): 3.05 min, m/z 419.6 [M+H]⁺ (45%) and 3.07 min, m/z 419.6 [M+H]⁺ (54%); ¹HNMR (400 MHz, DMSO-d₆): δ 8.88 (d, J=2.4 Hz, 1H), 8.28-8.25 (m, 1H), 7.81-7.72 (m, 2H), 7.62-7.53 (m, 3H), 5.60-5.52 (m, 1H), 5.39-5.34 (m, 1H), 4.07-4.01 (m, 1H), 3.97-3.82 (m, 3H), 2.65 (s, 3H), 2.45-2.37 (m, 1H), 2.15-2.04 (m, 1H), 1.63 (d, J=6.8 Hz, 3H)

The following compound was prepared in a similar manner, starting from the corresponding amine.

| Compound | Structure | Analytical data |
|---|---|---|
| 10 | 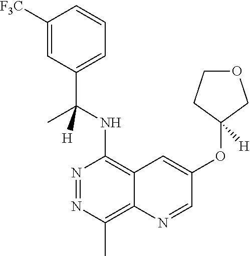 [SMILES: Cc1nnc(N[C@H](C)c2cccc(C(F)(F)F)c2)c2cc(O[C@H]3CCOC3)cnc12] | UPLC-MS (ES⁺, Long acidic): 3.08 min, m/z 419.6 [M + H]⁺ (96%) ¹H NMR (400 MHz, DMSO-d₆): δ 8.88 (d, J = 2.4Hz, 1H), 8.26 (d, J = 2.8Hz, 1H), 7.78 (br s, 1H), 7.76-7.73 (m, 1H), 7.62-7.52 (m, 3H), 5.60-5.52 (m, 1H), 5.39-5.34 (m, 1H), 4.03 (dd, J = 4.4, 10.4Hz, 1H), 3.96-3.90 (m, 2H), 3.89-3.82 (m, 1H), 2.65 (s, 3H), 2.45-2.37 (m, 1H), 2.15-2.04 (m, 1H), 1.63 (d, J = 7.2Hz, 3H) |

### Example 4. Synthesis of 3-(cyclopenten-1-yl)-5-[[(1R)-1-[3-(difluoromethyl)phenyl]ethyl]amino]-1,8-dimethyl-pyrido[2,3-d]pyridazin-2-one (11)

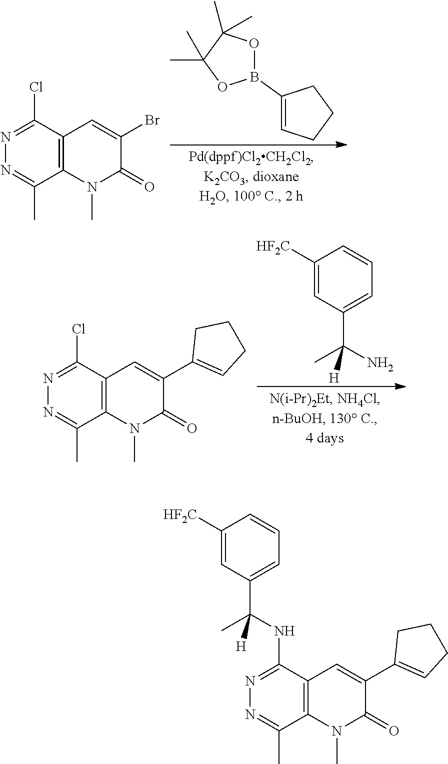

Step 1: 2-(1-cyclopenten-1-yl)-4,4,5,5-tetramethyl-1,3,2-dioxaborolane (515.72 mg, 2.66 mmol), potassium carbonate (667.75 mg, 4.83 mmol) and 2-(1-cyclopenten-1-yl)-4,4,5,5-tetramethyl-1,3,2-dioxaborolane (515.72 mg, 2.66 mmol) were mixed in 1,4-dioxane (5 mL) and water (1 mL) and degassed with nitrogen for 5 minutes. [1,1′-Bis(diphenylphosphino)ferrocene]Palladium(II) chloride dichloromethane complex (197.28 mg, 0.2400 mmol) was added and the reaction was heated to 100° C. for 2 hours. The reaction was concentrated to dryness and the residue was then purified by flash column chromatography (25 g, eluent ethyl acetate in petroleum ether 30-80%) to afford 5-chloro-3-(cyclopenten-1-yl)-1,8-dimethyl-pyrido[2,3-d]pyridazin-2-one (355 mg, 1.2875 mmol, 53.2% yield) as a beige solid.

UPLC-MS (ES+, Short acidic): 1.77 min, m/z 276/278 [M+H]⁺ (86%)

¹H NMR (400 MHz, CDCl₃): δ 7.83 (s, 1H), 7.40 (s, 1H), 3.92 (s, 3H), 3.10 (s, 3H), 2.76-2.84 (m, 2H), 2.63-2.72 (m, 2H), 2.03 (quint, J=7.68 Hz, 2H)

Step 2: A stirred solution of 5-chloro-3-(cyclopenten-1-yl)-1,8-dimethyl-pyrido[2,3-d]pyridazin-2-one (200.mg, 0.7300 mmol), (1R)-1-[3-(difluoromethyl)phenyl]ethylamine (310.43 uL, 2.18 mmol) and N,N-diisopropylethylamine (252.86 uL, 1.45 mmol) in n-butanol (2 mL) was heated to 130° C. in a sealed vial for 4 days. The reaction was concentrated to dryness and the residue was partitioned between dichloromethane and water. The aqueous layer was extracted with dichloromethane (3×). The organic phase was washed with brine, passed through a phase separator and concentrated under reduced pressure. The residue was then purified by flash column chromatography several times: (12 g, eluent methanol in dichloromethane 0-5%), (12 g, eluent ethyl acetate in petroleum ether 30-100%) and (12 g, eluent methanol in dichloromethane 2%). The product was then passed through an SCX-2 (2 g) cartridge to afford 3-(cyclopenten-1-yl)-5-[[(1R)-1-[3-(difluoromethyl)phenyl]ethyl]amino]-1,8-dimethyl-pyrido[2,3-d]pyridazin-2-one (70 mg, 0.1705 mmol, 23.5% yield) as a brown gummy solid.

UPLC-MS (ES⁺, Short acidic): 1.69 min, m/z 411.2 [M+H]⁺ (100%)

UPLC-MS (ES⁺, Long acidic): 3.63 min, m/z 411.5 [M+H]⁺ (98%)

¹H NMR (400 MHz, DMSO-d₆): δ 8.08 (s, 1H), 7.61 (s, 1H), 7.59 (d, J=8.7 Hz, 1H), 7.52 (d, J=7.4 Hz, 1H), 7.44 (t, J=7.6 Hz, 1H), 7.39 (d, J=7.6 Hz, 1H), 7.19 (s, 1H), 7.0 (t, J=56.0 Hz, 1H), 5.51 (quint, J=7.1 Hz, 1H), 3.76 (s, 3H), 2.76-2.87 (m, 5H), 2.54-2.61 (m, 2H), 1.94 (quint, J=7.5 Hz, 2H), 1.58 (d, J=7.0 Hz, 3H).

The following examples were prepared in a similar manner, starting from the corresponding amine.

| Compound | Structure | Analytical data |
|---|---|---|
| 12 | 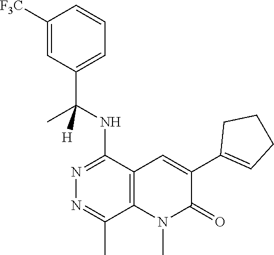 [SMILES: Cc1nnc(N[C@H](C)c2cccc(C(F)(F)F)c2)c2cc(C3=CCCC3)c(=O)n(C)c12] | UPLC-MS (ES+, Long acidic): 4.00 min, m/z 429.6 [M + H]⁺ (100%) ¹H NMR (400 MHz, DMSO-d₆): 8.06 (s, 1H), 7.76 (s, 1H), 7.69-7.75 (m, 1H), 7.50-7.58 (m, 3H), 7.19 (s, 1H), 5.52 (quint, J = 7.1Hz, 1H), 3.76 (s, 3H), 2.76-2.88 (m, 5H), 2.54-2.61 (m, 2H), 1.94 (quint, J = 7.4Hz, 2H), 1.60 (d, J = 7.1Hz, 3H) |

### Example 5. Synthesis of 6-cyclopropyl-1-methyl-4-[[(1R)-1-[3 (trifluoromethyl)phenyl]ethyl]amino]pyrido[3,4-d]pyridazin-7-one (13)

Compound 13 was prepared from intermediate described in Example 2 using the following procedure:

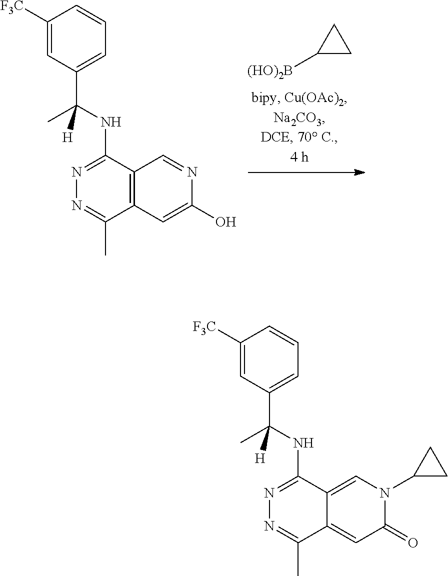 [SMILES: Cc1nnc(N[C@H](C)c2cccc(C(F)(F)F)c2)c2cnc(O)cc12 ; OB(O)C1CC1 ; Cc1nnc(N[C@H](C)c2cccc(C(F)(F)F)c2)c2cn(C3CC3)c(=O)cc12]

A mixture of 1-methyl-4-[[(1R)-1-[3-(trifluoromethyl)phenyl]ethyl]amino]-6H-pyrido[3,4-d]pyridazin-7-one (30 mg, 0.09 mmol), copper(II) acetate (16 mg, 0.09 mmol), cyclopropylboronic acid (15 mg, 0.17 mmol), 2,2′-bipyridyl (14 mg, 0.09 mmol), sodium carbonate (20 mg, 0.19 mmol) in DCE (0.5 mL). Air was bubbled in the solution, the vial was sealed, and the reaction was stirred at 70° C. for 4 h. The reaction mixture was concentrated and the residue was purified by column chromatography (4 g, eluting in 0-100% EtOAc in petroleum ether followed by 0-8% MeOH in DCM) to afford 6-cyclopropyl-1-methyl-4-[[(1R)-1-[3-(trifluoromethyl)phenyl]ethyl]amino]pyrido[3,4-d]pyridazin-7-one (8 mg, 0.02 mmol, 23.9% yield) as a yellow solid.

UPLC-MS (ES+, Long acidic): 2.67 min, m/z 389.3 [M+H]⁺ (>91%).

¹H-NMR (400 MHz, DMSO-d₆): δ 8.99 (s, 1H), 7.75-7.69 (m, 3H), 7.60-7.51 (m, 2H), 6.51 (s, 1H), 5.53-5.46 (m, 1H), 3.68-3.58 (m, 1H), 2.35 (s, 3H), 1.56 (d, J=6.8 Hz, 3H), 1.20-1.02 (m, 4H)

### Example 6. Synthesis of 1-methyl-6-(1-methylcyclopropyl)-4-[[rac-(1R)-1-[3-(trifluoromethyl)phenyl]ethyl]amino]pyrido[3,4-d]pyridazin-7-one (14)

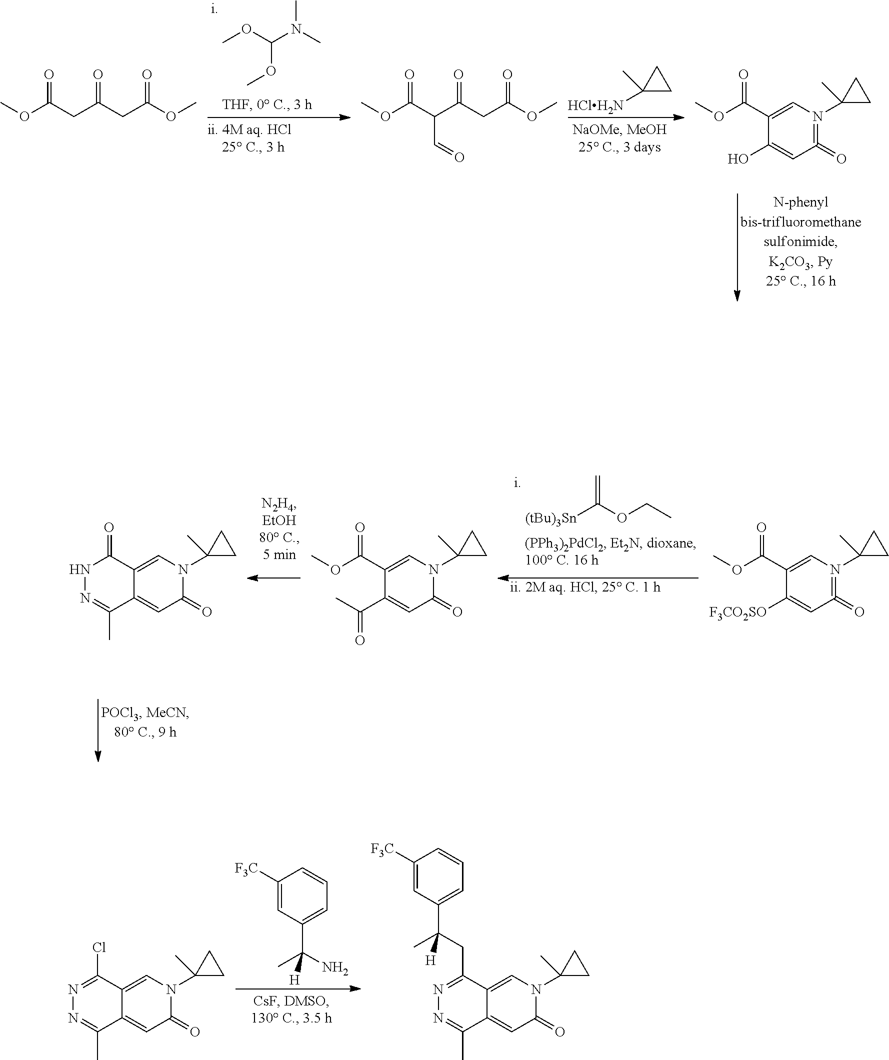

Step 1: To a solution of dimethyl-1,3-acetonedicarboxylate (2.0 g, 11.5 mmol) in THF (15 mL) at 0° C. was added N, N-dimethylformamide dimethyl acetal (1.52 mL, 11.5 mmol) dropwise and the reaction was stirred at 0° C. for 3 hours. 4M aq. HCl (5.74 mL, 23.0 mmol) was added and the reaction was warmed to rt and stirred for 3 hours. EtOAc was added (×2) and the two phases were separated. The organic extracts were washed with brine, dried over Na₂SO₄ and concentrated in vacuo to afford dimethyl 2-formyl-3-oxo-pentanedioate (2.13 g, 10.5 mmol, 91.7% yield) as a yellow oil.

UPLC-MS (ES⁺, short acidic): 1.48 min, m/z 203.0 [M+H]⁺. [00202]¹H NMR (400 MHz, CDCl₃): δ 8.99 (d, J=8.0 Hz, 1H), 3.95 (s, 2H), 3.78 (s, 3H), 3.76-3.74 (m, 4H).

Step 2: To a solution of dimethyl 2-formyl-3-oxo-pentanedioate (2.08 g, 10.3 mmol) in methanol (6 mL) was added 1-methylcyclopropanamine hydrochloride (1:1) (963 mg, 8.95 mmol) and the reaction mixture was stirred at rt overnight. A solution of sodium methoxide (1.04 g, 19.2 mmol) in Methanol (2.2 mL) was then added slowly and the reaction mixture was stirred at rt over the weekend. Water was added followed by the addition of aq. HCl to pH ˜3-4. The crude was extract with EtOAc (×3). The organic phases were combined, washed with brine, dried over Na₂SO₄ and the solvent removed under reduce pressure. The crude was purified by column chromatography (25 g, eluting in 0-100% EtOAc in petroleum ether) to afford methyl 4-hydroxy-1-(1-methylcyclopropyl)-6-oxo-pyridine-3-carboxylate (771 mg, 3.45 mmol, 38.6% yield) as a yellow solid.

UPLC-MS (ES⁺, Short acidic): 1.27 min, m/z 224.1 [M+H]⁺ (97%)

¹H NMR (400 MHz, CDCl₃): δ 10.45 (s, 1H), 8.22 (s, 1H), 5.88 (s, 1H), 3.92 (s, 3H), 1.53 (s, 3H), 1.08-0.98 (m, 4H).

Step 3: To a solution of methyl 4-hydroxy-1-(1-methylcyclopropyl)-6-oxo-pyridine-3-carboxylate (771 mg, 3.45 mmol) in pyridine (5.6 mL) was added potassium carbonate (836 mg, 6.05 mmol) and N-phenyl bis-(trifluoromethanesulfonimide) (2.16 g, 6.05 mmol). The solution was stirred at room temperature overnight. The solvents were then removed in vacuo, and the reaction mixture was partitioned between 2M aq. K₂CO₃ and EtOAc, and the aqueous layer was extracted with EtOAc (×2). The organic extracts were combined and dried over Na₂SO₄, filtered and then concentrated in vacuo to afford methyl 1-(1-methylcyclopropyl)-6-oxo-4-(trifluoromethylsulfonyloxy)pyridine-3-carboxylate (1.23 g, 3.45 mmol, quantitative yield) as a brown oil.

UPLC-MS (ES⁺, Short acidic): 1.85 min, m/z 356.1 [M+H]⁺ (93%)

Step 4: A solution of tributyl(1-ethoxyvinyl)tin (1.4 mL, 4.15 mmol), methyl 1-(1-methylcyclopropyl)-6-oxo-4-(trifluoromethylsulfonyloxy)pyridine-3-carboxylate (1.23 g, 3.45 mmol), triethylamine (1.2 mL, 8.64 mmol) in dry 1,4-dioxane (11 mL) was degassed for 5 min in a vial. Bis(triphenylphosphine)palladium(II) dichloride (242 mg, 0.35 mmol) was the added and the solution degassed for a further 5 mins. The vial was sealed and the reaction was heated at 100° C. overnight. The reaction was cooled down to rt and 2M aq. HCl (8.63 mL, 17.3 mmol) was added and stirred for 1 h. Water and EtOAc were added, The two phases were separated and the aqueous layer was re-extracted with EtOAc (×3). The combined organic extracts were dried over Na₂SO₄ and concentrated in vacuo. The crude material was purified by column chromatography (25 g, eluting in 0-100% EtOAc in petroleum ether) to afford methyl 4-acetyl-1-(1-methylcyclopropyl)-6-oxo-pyridine-3-carboxylate (419 mg, 1.68 mmol, 48.7% yield) as a yellow solid.

UPLC-MS (ES⁺, Short acidic): 1.42 min, m/z 250.1 [M+H]⁺ (100%)

¹H NMR (400 MHz, CDCl₃): δ 8.25 (s, 1H), 6.29 (s, 1H), 3.84 (s, 3H), 2.46 (s, 3H), 1.54 (s, 3H), 1.06 (s, 4H).

Step 5: Methyl 4-acetyl-1-(1-methylcyclopropyl)-6-oxo-pyridine-3-carboxylate (419.mg, 1.68 mmol) and hydrazine hydrate (98.13 uL, 2.02 mmol) were mixed in ethanol (6.2 mL).

The reaction mixture was heated at 80° C. for 5 min. The reaction mixture was concentrated in vacuo. The solid was filtered, washed with MTBE and dried to afford 1-methyl-6-(1-methylcyclopropyl)-3H-pyrido[3,4-d]pyridazine-4,7-dione (325 mg, 1.4054 mmol, 83.6% yield) as an off white solid.

UPLC-MS (ES+, Short acidic): 1.08 min, m/z 232.1 [M+H]⁺ (>91%)

¹H NMR (400 MHz, DMSO-d₆): δ 11.90 (s, 1H), 8.61 (d, J=0.4 Hz, 1H), 6.54 (d, J=0.4 Hz, 1H), 2.27 (s, 3H), 1.48 (s, 3H), 1.09-1.05 (m, 2H), 1.03-0.99 (m, 2H).

Step 6: To a solution of 1-methyl-6-(1-methylcyclopropyl)-3H-pyrido[3,4-d]pyridazine-4,7-dione (325.mg, 1.41 mmol) in MeCN (5.5 mL) was added phosphorus oxychloride (458.5 uL, 4.92 mmol). The reaction mixture was heated at 80° C. for 9 h. The reaction mixture was concentrated in vacuo. The residue was then taken up in EtOAc and sat. aq. solution of NaHCO₃. The two phases were separated and the aqueous layer was extracted with EtOAc. The combined organic extracts were dried over Na₂SO₄ and concentrated in vacuo to afford 4-chloro-1-methyl-6-(1-methylcyclopropyl)pyrido[3,4-d]pyridazin-7-one (98 mg, 0.3925 mmol, 27.9% yield) as a brown solid. The material was used in the next step without further purification.

UPLC-MS (ES+, Short acidic): 1.18 min, m/z 250.1 [M+H]⁺ (55%)

¹H NMR (400 MHz, DMSO-d₆): δ 8.86 (s, 1H), 6.81 (s, 1H), 2.57 (s, 3H), 1.54 (s, 3H), 1.27-1.23 (m, 2H), 1.09-1.06 (m, 2H).

Step 7: To a vial was added 4-chloro-1-methyl-6-(1-methylcyclopropyl)pyrido[3,4-d]pyridazin-7-one (49 mg, 0.2 mmol) and (1R)-1-[3-(trifluoromethyl)phenyl]ethylamine (30.94 uL, 0.2 mmol) in DMSO (1 mL). Cesium Fluoride (44.71 mg, 0.29 mmol) was added and the vial was sealed. The reaction mixture was heated at 130° C. for 3.5 h. The reaction mixture was cooled to rt. Water and EtOAc were added. The two phases were separated. The aqueous was re-extracted with EtOAc (2×). The combined organic extracts were dried over Na₂SO₄ and concentrated in vacuo. The crude material was loaded into an SCX, which was flushed with MeOH and then eluted with 1N NH₃ in methanol. The crude residue was then purified by column chromatography (4 g, eluting in 0-100% EtOAc in petroleum ether followed by 0-10% MeOH in DCM). The product was further purified by prep HPLC (middle method). Fraction containing the product was loaded into an SCX cartridge, which was washed with MeOH and then eluted in 1N NH₃ in methanol to give 1-methyl-6-(1-methylcyclopropyl)-4-[[rac-(1R)-1-[3-(trifluoromethyl)phenyl]ethyl]amino]pyrido[3,4-d]pyridazin-7-one (4 mg, 0.0099 mmol, 5.1% yield) as a yellow solid.

UPLC-MS (ES⁺, Long acidic): 2.95 min, m/z 403.4 [M+H]⁺ (92%). [00219]¹H NMR (400 MHz, DMSO-d₆): δ 9.22 (s, 1H), 7.76-7.70 (m, 3H), 7.59-7.53 (m, 2H), 6.49 (s, 1H), 5.51-5.44 (m, 1H), 2.34 (s, 3H), 1.57 (d, J=7.2 Hz, 3H), 1.54 (s, 3H), 1.16 (br s, 2H), 1.08 (br s, 2H).

### Example 7. 5-methyl-2-[(3S)-tetrahydrofuran-3-yl]oxy-N-[1-[3-(trifluoromethyl)phenyl]ethyl]pyrido[2,3-d]pyridazin-8-amine (15)

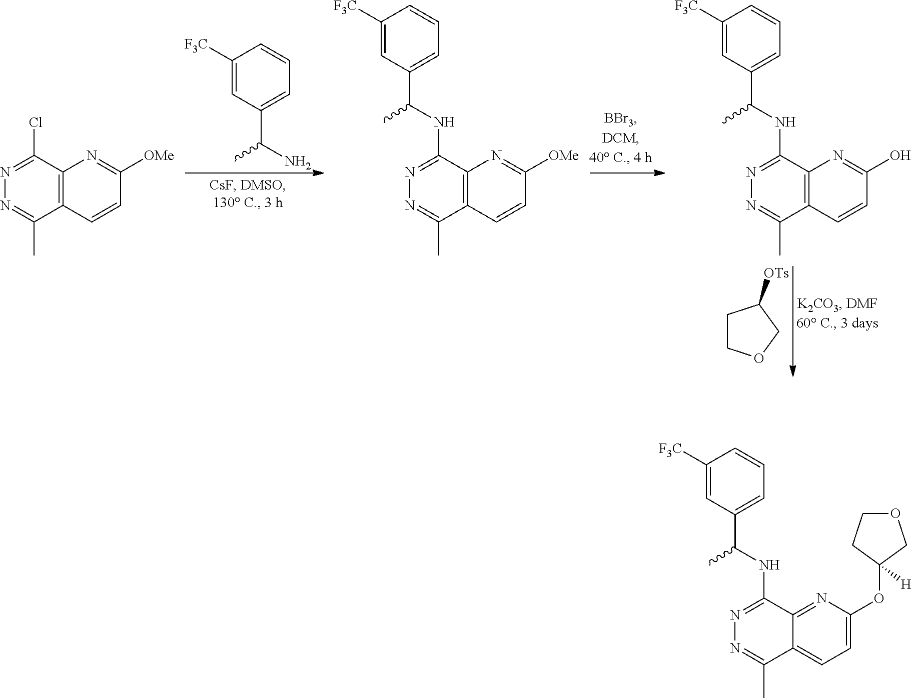

Step 1: To a vial was added 8-chloro-2-methoxy-5-methyl-pyrido[2,3-d]pyridazine (125 mg, 0.60 mmol) and alpha-methyl-3-(trifluoromethyl)benzylamine (94 μL, 0.60 mmol) in DMSO (3 mL). Cesium fluoride (136 mg, 0.89 mmol) was added and the vial was sealed. The reaction mixture was heated at 130° C. for 3 days. The reaction was cooled to rt and partitioned between water and EtOAc. The two phases were separated and the aqueous was extracted with EtOAc (×3). The combined organic extracts were dried over Na₂SO₄, filtered and concentrated in vacuo. The crude material was purified by column chromatography (4 g, eluting in 0-100% EtOAc in petroleum ether) to afford 2-methoxy-5-methyl-N-[1-[3-(trifluoromethyl)phenyl]ethyl]pyrido[2,3-d]pyridazin-8-amine (73.8 mg, 0.20 mmol, 34.2% yield) as a yellow oil.

UPLC-MS (ES⁺, Short acidic): 1.61 min, m/z 363.1 [M+H]⁺ (96%)

¹H NMR (400 MHz, DMSO-d₆): δ 8.32 (d, J=8.9 Hz, 1H), 7.84 (s, 1H), 7.81-7.77 (m, 1H), 7.57-7.54 (m, 2H), 7.36 (d, J=8.9 Hz, 1H), 7.18 (d, J=7.9 Hz, 1H), 5.53-5.45 (m, 1H), 4.17 (s, 3H), 2.61 (s, 3H), 1.66 (d, J=7.0 Hz, 3H)

Step 2: To a solution of 2-methoxy-5-methyl-N-[1-[3-(trifluoromethyl)phenyl]ethyl]pyrido[2,3-d]pyridazin-8-amine (86.3 mg, 0.24 mmol) in DCM at 0° C. was added boron tribromide (1M in DCM, 0.72 mL, 0.72 mmol) and the reaction was heated to 40° C. for 4 h. The reaction was then cooled to 0° C., quenched with MeOH and concentrated under reduced pressure to afford 5-methyl-8-[1-[3-(trifluoromethyl)phenyl]ethylamino]pyrido[2,3-d]pyridazin-2-ol (83 mg, 0.24 mmol, 100% yield) as a brown solid. The crude product was used in the next step without further purification.

UPLC-MS (ES+, Short acidic): 1.40 min, m/z 349.1 [M+H]⁺ (84%)

Step 3: 5-methyl-8-[1-[3-(trifluoromethyl)phenyl]ethylamino]pyrido[2,3-d]pyridazin-2-ol (83 mg, 0.24 mmol), potassium carbonate (49.4 mg, 0.36 mmol) and [(3R)-tetrahydrofuran-3-yl]4-methylbenzenesulfonate (92.3 mg, 0.38 mmol) were mixed in DMF (1.6 mL). The reaction mixture was heated to 60° C. for 3 days. The reaction mixture was concentrated under reduced pressure and purified by column chromatography (4 g, eluting in 0-100% EtOAc in petroleum ether). Fractions containing the product were passed through an SCX, which was flush with MeOH and then with NH₃ 1N in MeOH to give 5-methyl-2-[(3S)-tetrahydrofuran-3-yl]oxy-N-[1-[3-(trifluoromethyl)phenyl]ethyl]pyrido[2,3-d]pyridazin-8-amine (12.7 mg, 0.03 mmol, 12.7% yield) as a green solid.

UPLC-MS (ES⁺, Long acidic): 3.11 min, m/z 419.8 [M+H]⁺ (31%), 3.13 min, m/z 419.8 [M+H]⁺ (67%).

¹H NMR (400 MHz, DMSO-d₆) δ 8.34 (d, J=8.9 Hz, 1H), 7.83 (s, 1H), 7.80-7.76 (m, 1H), 7.58-7.54 (m, 2H), 7.36 (d, J=8.9 Hz, 1H), 7.20-7.17 (m, 1H), 6.07-6.04 (m, 1H), 5.56-5.47 (m, 1H), 4.11 (dd, J=4.9, 10.4 Hz, 1H), 3.95-3.87 (m, 1H), 3.87-3.81 (m, 2H), 2.61 (s, 3H), 2.45-2.37 (m, 1H), 2.12-2.05 (m, 1H), 1.66 (d, J=7.0 Hz, 3H).

### Example 8. 1,8-dimethyl-3-(1,2,3,6-tetrahydropyridin-5-yl)-5-[[(1R)-1-[3-(trifluoromethyl)phenyl]ethyl]amino]pyrido[2,3-d]pyridazin-2-one (16)

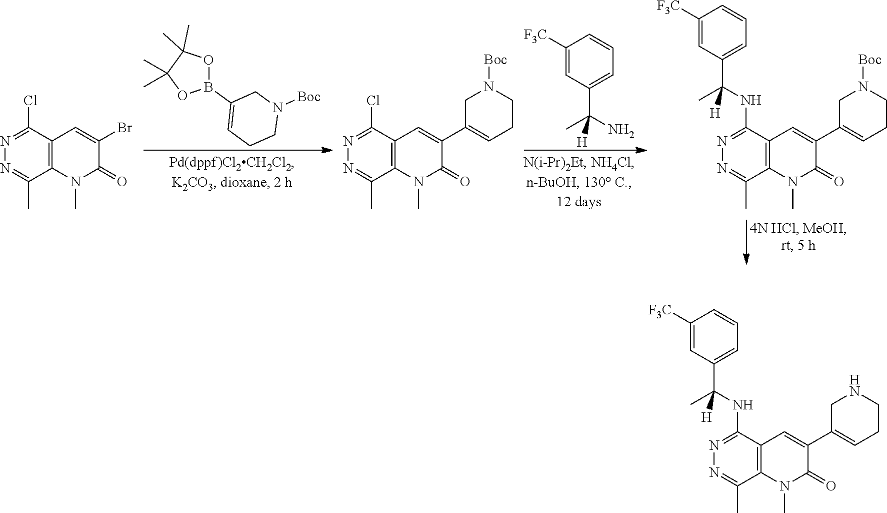 [SMILES: Cc1nnc(Cl)c2cc(Br)c(=O)n(C)c12 ; CC(C)(C)OC(=O)N1CCC=C(B2OC(C)(C)C(C)(C)O2)C1 ; Cc1nnc(Cl)c2cc(C3=CCCN(C(=O)OC(C)(C)C)C3)c(=O)n(C)c12 ; C[C@@H](N)c1cccc(C(F)(F)F)c1 ; Cc1nnc(N[C@H](C)c2cccc(C(F)(F)F)c2)c2cc(C3=CCCN(C(=O)OC(C)(C)C)C3)c(=O)n(C)c12 ; Cc1nnc(N[C@H](C)c2cccc(C(F)(F)F)c2)c2cc(C3=CCCNC3)c(=O)n(C)c12]

Step 1: 3-bromo-5-chloro-1,8-dimethyl-pyrido[2,3-d]pyridazin-2-one (750 mg, 2.60 mmol), potassium carbonate (719 mg, 5.19 mmol) and N-Boc-1,2,3,6-tetrahydropyridine-4-boronic acid pinacol ester (965 mg, 3.12 mmol) were mixed in 1,4-dioxane (5 mL) and water (1 mL) and degassed with nitrogen for 5 minutes. [1,1′-Bis(diphenylphosphino)ferrocene]palladium(II) chloride dichloromethane complex (212 mg, 0.26 mmol) was added, the reaction was degassed for another 5 minutes and then heated to 100° C. for 2 hours. The reaction was combined and concentrated to dryness. The residue was partitioned between DCM and water. The aqueous layer was extracted with DCM (×4). The organic phase was washed with brine, passed through a phase separator and concentrated under reduced pressure. The residue was then purified by column chromatography (12 g, eluting in 20-100% EtOAc in petroleum ether) to afford tert-butyl 4-{5-chloro-1,8-dimethyl-2-oxo-1H,2H-pyrido[2,3-d]pyridazin-3-yl}-1,2,3,6-tetrahydropyridine-1-carboxylate (751 mg, 1.9214 mmol, 73.9% yield) as a yellow solid.

UPLC-MS (ES⁺, Short acidic): 1.75 min, m/z 391.2/392.3 [M+H]⁺ (91%)

¹H NMR (400 MHz, CDCl₃): δ 7.92 (s, 1H), 6.93-6.43 (m, 1H), 4.33 (s, 2H), 3.89 (s, 3H), 3.59 (t, J=5.8 Hz, 2H), 3.11 (s, 3H), 2.39 (s, 2H), 1.49 (s, 9H)

Step 2: A stirred solution of tert-butyl 5-(5-chloro-1,8-dimethyl-2-oxo-pyrido[2,3-d]pyridazin-3-yl)-3,6-dihydro-2H-pyridine-1-carboxylate (750 mg, 1.92 mmol), (1R)-1-[3-(trifluoromethyl)phenyl]ethylamine (484 μL, 3.07 mmol) and N,N-diisopropylethylamine (668 μL, 3.84 mmol) in 1-butanol (4 mL) was heated to 130° C. in a sealed vial for 3 days. Further (1R)-1-[3-(trifluoromethyl)phenyl]ethylamine (303 μL, 1.92 mmol) was added and the reaction was heated to 130° C. for 9 days. The reaction was partitioned between DCM and water. The aqueous layer was extracted with DCM (×3). The organic phase was washed with brine, passed through a phase separator and concentrated under reduced pressure. The residue was then purified by column chromatography (12 g, eluting in 0-100% EtOAc in petroleum ether followed by 0-4% MeOH in DCM) to afford tert-butyl 5-[1,8-dimethyl-2-oxo-5-[[rac-(1R)-1-[3-(trifluoromethyl)phenyl]ethyl]amino]pyrido[2,3-d]pyridazin-3-yl]-3,6-dihydro-2H-pyridine-1-carboxylate (629 mg, 1.16 mmol, 60.3% yield) as a brown oil.

UPLC-MS (ES+, Long acidic): 3.95 min, m/z 544.5 [M+H]⁺ (84%)

Step 3: To a stirring solution of tert-butyl 5-[1,8-dimethyl-2-oxo-5-[[(1R)-1-[3-(trifluoromethyl)phenyl]ethyl]amino]pyrido[2,3-d]pyridazin-3-yl]-3,6-dihydro-2H-pyridine-1-carboxylate (629 mg, 1.16 mmol) in methanol (5 mL) was added HCl 4N in dioxane (2.03 mL, 8.10 mmol) was added and the reaction was stirred for 5 hours. The reaction was concentrated to dryness. The residue was taken up in MeOH, passed through SCX and flushed with NH₃ 1N in MeOH. The residue was then purified by column chromatography using as eluent a gradient 20-100% EtOAc in petroleum ether followed by 0-20% MeOH in DCM to afford 1,8-dimethyl-3-(1,2,3,6-tetrahydropyridin-5-yl)-5-[[(1R)-1-[3-(trifluoromethyl)phenyl]ethyl]amino]pyrido[2,3-d]pyridazin-2-one (352 mg, 0.79 mmol, 68.6% yield) as a yellow resin.

UPLC-MS (ES⁺, Long acidic): 2.53 min, m/z 444.2 [M+H]⁺ (98%)

¹H NMR (400 MHz, DMSO-d₆): δ 8.14 (s, 1H), 7.76 (s, 1H), 7.72 (d, J=6.1 Hz, 1H), 7.58-7.48 (m, 3H), 6.50-6.45 (m, 1H), 5.49 (quint, J=7.1 Hz, 1H), 3.72 (s, 3H), 3.64 (d, J=11.2 Hz, 2H), 2.89 (t, J=5.7 Hz, 2H), 2.79 (s, 3H), 2.26-2.18 (m, 2H), 1.58 (d, J=7.1 Hz, 3H). 1H hidden

### Example 9. 1,8-dimethyl-3-(3-piperidyl)-5-[[(1R)-1-[3-(trifluoromethyl)phenyl]ethyl]amino]pyrido[2,3-d]pyridazin-2-one (17)

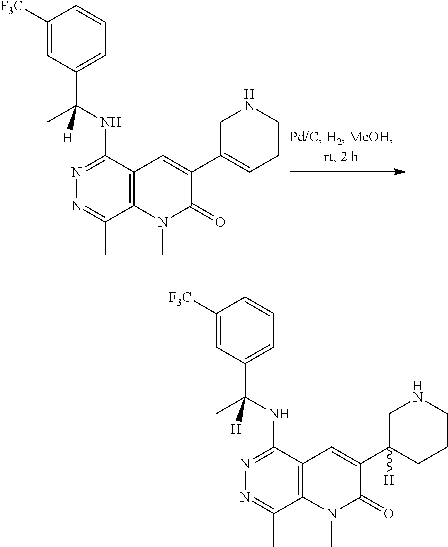 [SMILES: Cc1nnc(N[C@H](C)c2cccc(C(F)(F)F)c2)c2cc(C3=CCCNC3)c(=O)n(C)c12 ; Cc1nnc(N[C@H](C)c2cccc(C(F)(F)F)c2)c2cc(C3CCCNC3)c(=O)n(C)c12]

A solution of 1,8-dimethyl-3-(1,2,3,6-tetrahydropyridin-5-yl)-5-[[(1R)-1-[3-(trifluoromethyl)phenyl]ethyl]amino]pyrido[2,3-d]pyridazin-2-one (100 mg, 0.23 mmol—Example 16) in methanol (5 mL) with a few drops of acetic acid was degassed using vacuum and nitrogen (3×). Afterwards palladium, 10 wt. % on carbon powder, dry (24 mg, 0.02 mmol) was added and the flask was evacuated and back-filled with N₂ (3×) and finally with H₂ through the same process (3×). The reaction was stirred at rt for 2 hours. LCMS indicated full conversion. H2 was removed, the mixture was filtered through celite, washed with MeOH and then evaporated to dryness. The residue was taken up in MeOH and passed through a SCX cartridge (5 g) then purified by flash column chromatography (12 g, eluting in 0-20% MeOH in DCM) to afford 1,8-dimethyl-3-(3-piperidyl)-5-[[(1R)-1-[3-(trifluoromethyl)phenyl]ethyl]amino]pyrido[2,3-d]pyridazin-2-one (223 mg, 0.50 mmol, 76.5% yield) as a yellow solid.

UPLC-MS (ES+, Long acidic): 2.49 min+2.57 min, m/z 446.2 [M+H]⁺ (45%+52%)

¹H NMR (400 MHz, DMSO-d₆): δ 8.05 (s, 1H), 7.75 (s, 1H), 7.73-7.67 (m, 1H), 7.59-750 (m, 2H), 7.47 (d, J=7.2 Hz, 1H), 5.51 (quint, J=7.0 Hz, 1H), 3.74 (s, 3H), 3.14-2.95 (m, 3H), 2.79 (s, 3H), 2.60-2.52 (m, 2H), 1.95-1.82 (m, 1H), 1.79-1.44 (m, 6H), NH not observed

### Example 10. 3-(1-acetyl-3-piperidyl)-1,8-dimethyl-5-[[(1R)-1-[3-(trifluoromethyl)phenyl]ethyl]amino]pyrido[2,3-d]pyridazine-2-one (18)

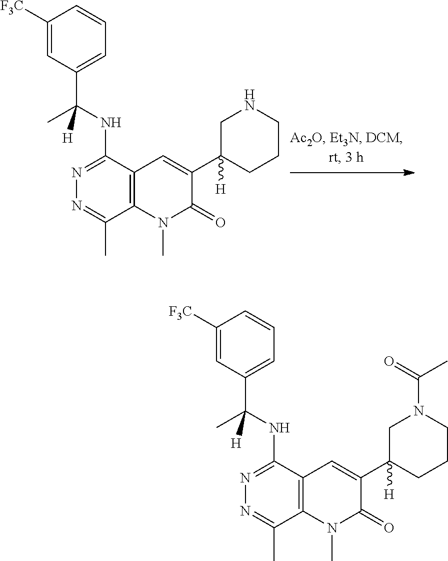 [SMILES: Cc1nnc(N[C@H](C)c2cccc(C(F)(F)F)c2)c2cc(C3CCCNC3)c(=O)n(C)c12 ; CC(=O)N1CCCC(c2cc3c(N[C@H](C)c4cccc(C(F)(F)F)c4)nnc(C)c3n(C)c2=O)C1]

To a stirring solution of 1,8-dimethyl-3-(3-piperidyl)-5-[[(1R)-1-[3-(trifluoromethyl)phenyl]ethyl]amino]pyrido[2,3-d]pyridazin-2-one (85 mg, 0.19 mmol—Example 17) in dry DCM (2 mL) was added triethylamine (53 μL, 0.38 mmol) followed by acetyl chloride (20 μL, 0.28 mmol). The reaction was then stirred at rt for 3 hours. The mixture was concentrated, passed through a SCX cartridge, which was flushed with MeOH and then eluted with NH₃ 1N in MeOH, then purified by column chromatography (4 g, eluting in 0-8% MeOH in DCM) to afford 3-(1-acetyl-3-piperidyl)-1,8-dimethyl-5-[[(1R)-1-[3-(trifluoromethyl)phenyl]ethyl]amino]-pyrido[2,3-d]pyridazine-2-one (92 mg, 0.19 mmol, 98.9% yield) as a pale yellow solid.

UPLC-MS (ES+, Short acidic): 1.47+1.49 min, m/z 488.5 [M+H]⁺ (43%+57%)

UPLC-MS (ES+, Long acidic): 3.17+3.21 min, m/z 488.4 [M+H]⁺ (46%+54%)

¹H NMR (400 MHz, DMSO-d₆): δ 8.14-8.07 (m, 1H), 7.75 (s, 1H), 7.72 (d, J=5.6 Hz, 1H), 7.59-7.52 (m, 2H), 7.52-7.40 (m, 1H), 5.58-5.43 (m, 1H), 4.64-4.44 (m, 1H), 4.01 (d, J=8.8 Hz, 0.5H), 3.90 (d, J=12.7 Hz, 0.5H), 3.76 (s, 1.5H), 3.74 (s, 1.5H), 3.17 (s, 0.5H), 3.14 (s, 0.5H), 3.06 (t, J=13.7 Hz, 0.5H), 2.99-2.90 (m, 1H), 2.82-2.75 (m, 3H), 2.71-2.61 (m, 0.5H), 2.08-2.03 (m, 3H), 2.01-1.86 (m, 1.5H), 1.86-1.67 (m, 2H), 1.61 (d, J=3.3 Hz, 1H), 1.59 (d, J=3.2 Hz, 1.5H)

### Example 11. 3-(1-acetyl-3,6-dihydro-2H-pyridin-4-yl)-1,8-dimethyl-5-[[rac-(1R)-1-[3-(trifluoromethyl)phenyl]ethyl]amino]pyrido[2,3-d]pyridazine-2-one (19)

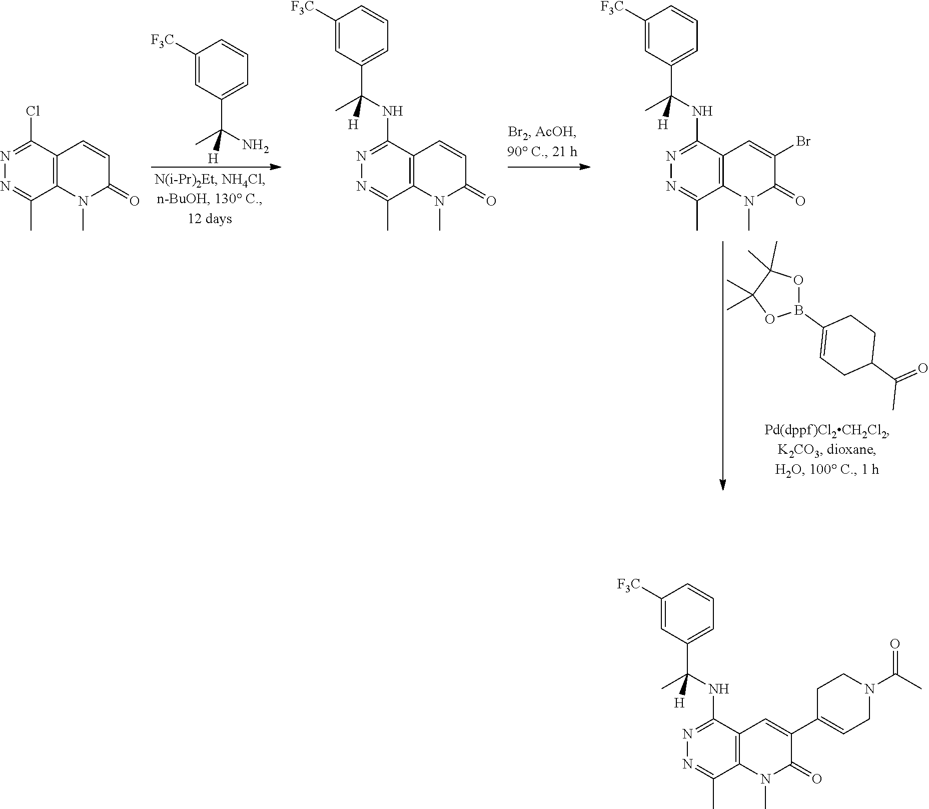

Step 1:_A stirred solution of 5-chloro-1,8-dimethyl-pyrido[2,3-d]pyridazine-2-one (850 mg, 4.05 mmol), (1R)-1-[3-(trifluoromethyl)phenyl]ethylamine (1.2 mL, 7.61 mmol), ammonium chloride (651 mg, 12.2 mmol) and N,N-diisopropylethylamine (2.12 mL, 12.2 mmol) in 1-butanol (6 mL) was heated to 130° C. in a sealed vial for 5 days. The reaction was partitioned between DCM and water. The aqueous layer was extracted with DCM (×4). The organic phase was washed with brine, passed through a phase separator and concentrated under reduced pressure. The residue was purified by column chromatography (12 g, eluting in 20-100% EtOAc in petroleum ether followed by 0-5% MeOH in DCM). The product was loaded into an SCX, which was flushed with MeOH then eluting with NH₃ 1N in MeOH to afford 1,8-dimethyl-5-[[rac-(1R)-1-[3-(trifluoromethyl)phenyl]ethyl]amino]pyrido[2,3-d]pyridazine-2-one (816 mg, 1.91 mmol, 47.2% yield) as an orange gum.

UPLC-MS (ES+, Short acidic): 1.42 min, m/z 363.3 [M+H]⁺ (85%)

¹H NMR (400 MHz, CDCl₃): δ 7.75 (d, J=9.7 Hz, 1H), 7.66-7.58 (m, 2H), 7.47 (d, J=7.8 Hz, 1H), 7.40 (t, J=7.6 Hz, 1H), 6.86 (d, J=9.7 Hz, 1H), 5.58 (quint, J=6.7 Hz, 1H), 5.25 (d, J=6.4 Hz, 1H), 3.83 (s, 3H), 2.93 (s, 3H), 1.62 (d, J=6.9 Hz, 3H)

Step 2: To a stirring solution of 1,8-dimethyl-5-[[rac-(1R)-1-[3-(trifluoromethyl)phenyl]ethyl]amino]pyrido[2,3-d]pyridazine-2-one (694 mg, 1.92 mmol) in acetic acid (6 mL) was added bromine (589 μL, 11.5 mmol). The mixture was heated to 90° C. in a sealed vial for 21 h. The reaction was concentrated and the residue was partitioned between DCM and water with Na₂S₂O₃. The aqueous layer was extracted with DCM (×3). The organic phase was washed with brine, passed through a phase separator, and concentrated under reduced pressure.

The residue was purified by column chromatography using as eluent a gradient 0-100% EtOAc in petroleum ether to afford 3-bromo-1,8-dimethyl-5-[[rac-(1R)-1-[3-(trifluoromethyl)phenyl]ethyl]-amino]pyrido[2,3-d]pyridazin-2-one (137 mg, 0.19 mmol, 9.7% yield) as an off-white solid.

UPLC-MS (ES+, Short acidic): 1.70 min, m/z 441/443 [M+H]⁺ (61%)

¹H NMR (400 MHz, CDCl₃): δ 8.12 (s, 1H), 7.68-7.61 (m, 2H), 7.50 (d, J=7.8 Hz, 1H), 7.44 (t, J=7.6 Hz, 1H), 5.61 (quint, J=6.7 Hz, 1H), 5.01 (d, J=6.5 Hz, 1H), 3.91 (s, 3H), 2.95 (s, 3H), 1.67 (d, J=6.8 Hz, 3H)

Step 3: 3-bromo-1,8-dimethyl-5-[[rac-(1R)-1-[3-(trifluoromethyl)phenyl]-ethyl]amino]pyrido[2,3-d]pyridazin-2-one (120 mg, 0.27 mmol), potassium carbonate (75 mg, 0.54 mmol) and 1-(4-(4,4,5,5-tetramethyl-1,3,2-dioxaborolan-2-yl)-5,6-dihydropyridin-1(2H)-yl)ethanone (68 mg, 0.27 mmol) were mixed in 1,4-dioxane (2 mL) and water (0.4 mL) and degassed with nitrogen for 5 minutes. [1,1′-Bis(diphenylphosphino)ferrocene]palladium(II) chloride dichloromethane complex (22 mg, 0.03 mmol) was then added and the reaction was heated to 100° C. for 1 h. The reaction was partitioned between DCM and water. The two phases were separated and the aqueous layer was re-extracted with DCM (×2). The combined organic extracts were washed with brine, passed through a phase separator and concentrated in vacuo. The residue was purified by column chromatography (4 g, eluting in 0-100% EtOAc in petroleum ether followed by 0-6% MeOH in DCM) to afford 3-(1-acetyl-3,6-dihydro-2H-pyridin-4-yl)-1,8-dimethyl-5-[[rac-(1R)-1-[3-(trifluoromethyl)phenyl]ethyl]amino]pyrido[2,3-d]pyridazin-2-one (80 mg, 0.16 mmol, 60.6% yield) as a yellow solid.

UPLC-MS (ES+, Long acidic): 3.07 min, m/z 486.4 [M+H]⁺ (98%)

¹H NMR (400 MHz, DMSO-d₆): δ 8.19 (s, 1H), 7.78 (s, 1H), 7.72 (d, J=6.6 Hz, 1H), 7.57-7.49 (m, 3H), 6.67 (s, 0.5H), 6.47 (s, 0.5H), 5.55-5.43 (m, 1H), 4.23-4.17 (m, 1H), 4.17-4.11 (m, 1H), 3.73 (s, 3H), 3.69-3.57 (m, 2H), 2.80 (s, 3H), 2.65-2.56 (m, 1H), 2.08 (s, 1.5H), 2.05 (s, 1.5H), 1.58 (d, J=7.0 Hz, 3H). 1H under solvent peak.

### Example 12. 1,8-dimethyl-5-[[rac-(1R)-1-[3-(difluoromethyl)-phenyl]ethyl]amino]-3-tetrahydropyran-4-yl-pyrido[2,3-d]pyridazin-2-one (20)

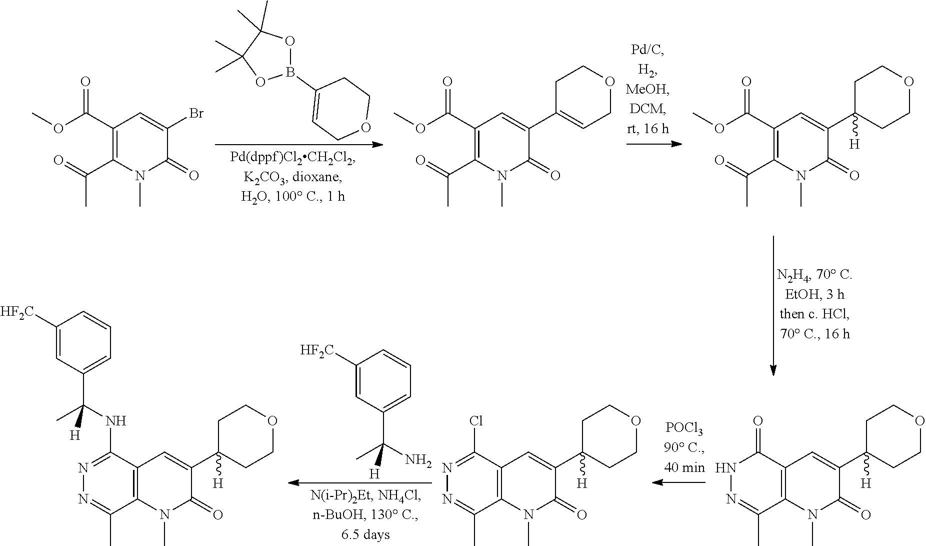

Step 1. Methyl 2-acetyl-5-bromo-1-methyl-6-oxo-pyridine-3-carboxylate (1 g, 3.47 mmol), potassium carbonate (959 mg, 6.94 mmol) and 3,6-dihydro-2H-pyran-4-boronic acid pinacol ester (875 mg, 4.17 mmol) were mixed in 1,4-dioxane (5 mL) and water (1 mL) and degassed with nitrogen for 10 minutes. [1,1′-Bis(diphenylphosphino)ferrocene]palladium(II) chloride dichloromethane complex (283 mg, 0.35 mmol) was added and the reaction was heated to 100° C. for 1 hour in a sealed vial. The reaction was partitioned between DCM and water. The two phases were separated and the aqueous was re-extracted with DCM (×2). The combined organic extracts were washed with brine, passed through a phase separator and concentrated in vacuo. The residue was then purified by flash column chromatography (12 g, eluting in 0-100% EtOAc in petroleum ether) to afford methyl 2-acetyl-5-(3,6-dihydro-2H-pyran-4-yl)-1-methyl-6-oxo-pyridine-3-carboxylate (820 mg, 2.82 mmol, 81.1% yield) as a yellow solid.

UPLC-MS (ES+, Short acidic): 1.43 min, m/z 292.3 [M+H]⁺ (100%)

Step 2. A solution of methyl 2-acetyl-5-(3,6-dihydro-2H-pyran-4-yl)-1-methyl-6-oxo-pyridine-3-carboxylate (815 mg, 2.80 mmol) in methanol (6 mL) and DCM (3 mL) was degassed using vacuum and nitrogen (3×). Afterwards palladium, 10 wt. % on carbon powder, dry (298 mg, 0.28 mmol) was added and the flask was evacuated and back-filled with N₂ (3×) and finally with H₂ through the same process (3×). The reaction was stirred at rt overnight. The mixture was filtered through celite, washed with MeOH and DCM, then evaporated to dryness to afford the crude methyl 2-acetyl-1-methyl-6-oxo-5-tetrahydropyran-4-yl-pyridine-3-carboxylate (851 mg, 2.90 mmol, 103.7% yield) as a white oily solid. The material was telescoped through to the next step without further purification.

UPLC-MS (ES+, Short acidic): 1.45 min, m/z 294.1 [M+H]⁺ (100%)

Step 3. To a stirring solution of methyl 2-acetyl-1-methyl-6-oxo-5-tetrahydropyran-4-yl-pyridine-3-carboxylate (850 mg, 2.90 mmol) in ethanol (7 mL) was added hydrazine hydrate (282 μL, 5.80 mmol). The mixture was heated to 70° C. for 3 h. LCMS showed no remaining starting material. Hydrogen Chloride (290 μL, 3.48 mmol) was added and the reaction was stirred at 70° C. overnight. The reaction was cooled down with an ice bath, filtered and washed with cold ethanol, then dried in the vacuum oven. The resulting solid was then purified by flash column chromatography (12 g, eluting in 0-10% MeOH in DCM) to afford 1,8-dimethyl-3-tetrahydropyran-4-yl-6H-pyrido[2,3-d]pyridazine-2,5-dione (321 mg, 1.17 mmol, 40.2% yield) as a white solid.

UPLC-MS (ES+, Short acidic): 1.19 min, m/z 276.1 [M+H]⁺ (95%)

¹H NMR (400 MHz, DMSO-d₆): δ 12.86 (s, 1H), 7.81 (s, 1H), 3.95 (dd, J=3.6, 10.9 Hz, 2H), 3.78 (s, 3H), 3.45 (t, J=10.2 Hz, 2H), 3.00 (tt, J=3.1, 11.9 Hz, 1H), 2.68 (s, 3H), 1.75 (d, J=12.5 Hz, 2H), 1.56 (qd, J=4.1, 12.4 Hz, 2H)

Step 4. 1,8-dimethyl-3-tetrahydropyran-4-yl-6H-pyrido[2,3-d]pyridazine-2,5-dione (320 mg, 1.16 mmol) in phosphorus oxychloride (2.5 mL, 26.8 mmol) was heated to 90° C. in a sealed vial for 40 min. The reaction was concentrated to dryness. Ice was added and aq. Na₂CO₃ was added to adjust to neutral pH. The aqueous layer was extracted with DCM (×7). The organic phase was passed through a phase separator and concentrated under reduced pressure to afford 5-chloro-1,8-dimethyl-3-tetrahydropyran-4-yl-pyrido[2,3-d]pyridazin-2-one (335 mg, 1.14 mmol, 98.1% yield) as a brown gum.

UPLC-MS (ES+, Short acidic): 1.31 min, m/z 294.1/295.9 [M+H]⁺ (94%)

Step 5. A stirred solution of 5-chloro-1,8-dimethyl-3-tetrahydropyran-4-yl-pyrido[2,3-d]pyridazin-2-one (335 mg, 1.14 mmol), (1R)-1-[3-(difluoromethyl)phenyl]ethylamine (312 mg, 1.82 mmol) and N,N-diisopropylethylamine (596 μL, 3.42 mmol) in 1-butanol (5 mL) was heated to 140° C. in a sealed vial for 6.5 days. The reaction was partitioned between DCM and water. The two phases were separated and the aqueous layer was re-extracted with DCM (×3). The combined organic extracts were washed with brine, passed through a phase separator, and concentrated in vacuo. The residue was purified using flash column chromatography (4 g, eluting in 0-100% EtOAc in petroleum ether followed by 0-5% MeOH in DCM) and then by reverse phase chromatography (4 g, eluting in 0-100% MeCN+0.1% formic acid in water+0.1% formic acid) to afford 1,8-dimethyl-5-[[rac-(1R)-1-[3-(difluoromethyl)phenyl]ethyl]amino]-3-tetrahydropyran-4-yl-pyrido[2,3-d]pyridazin-2-one (150 mg, 0.35 mmol, 30.7% yield) as a pale yellow solid.

UPLC-MS (ES+, Long acidic): 2.90 min, m/z 429.5 [M+H]⁺ (100%)

¹H NMR (400 MHz, DMSO-d₆): δ 8.14 (s, 1H), 7.67-7.53 (m, 2H), 7.50-7.35 (m, 3H), 7.01 (t, J=55.9 Hz, 1H), 5.49 (quint, J=7.0 Hz, 1H), 4.07-3.92 (m, 2H), 3.74 (s, 3H), 3.54-3.39 (m, 2H), 3.18-3.02 (m, 1H), 2.79 (s, 3H), 1.85-1.66 (m, 4H), 1.58 (d, J=7.0 Hz, 3H)

### Example 13. 1,8-dimethyl-3-(1-methyl-4-piperidyl)-5-[[rac-(1R)-1-[3-(difluoromethyl)phenyl]ethyl]amino]pyrido[2,3-d]pyridazin-2-one (21)

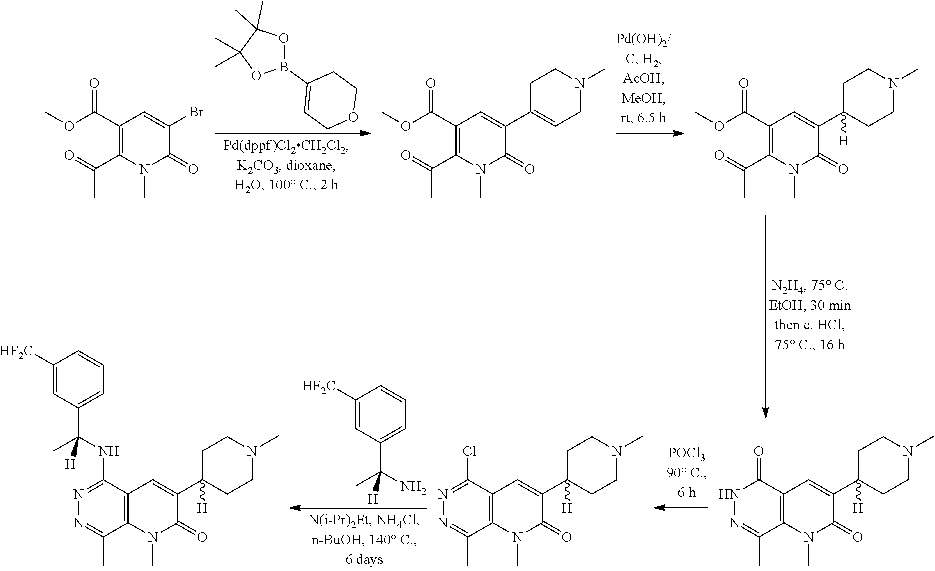

Step 1. Methyl 2-acetyl-5-bromo-1-methyl-6-oxo-pyridine-3-carboxylate (553 mg, 1.92 mmol), potassium carbonate (531 mg, 3.84 mmol) and 1-methyl-4-(4,4,5,5-tetramethyl-1,3,2-dioxaborolan-2-yl)-1,2,3,6-tetrahydro pyridine (514 mg, 2.30 mmol) were mixed in 1,4-dioxane (4 mL) and water (0.8 mL) and degassed with nitrogen for 10 minutes. [1,1′-Bis(diphenylphosphino)ferrocene]palladium(II) chloride dichloromethane complex (157 mg, 0.19 mmol) was added and the reaction was heated to 100° C. for 2 hours in a sealed vial. The reaction was partitioned between DCM and water. The two phases were separated and the aqueous layer was re-extracted with DCM (×3). The combined organic extracts were washed with brine, passed through a phase separator and concentrated in vacuo. The residue was then purified by flash column chromatography (12 g, eluting with 0-20% MeOH in DCM) to afford methyl 2-acetyl-1-methyl-5-(1-methyl-3,6-dihydro-2H-pyridin-4-yl)-6-oxo-pyridine-3-carboxylate (588 mg, 1.93 mmol, 100% yield) as a brown oil.

UPLC-MS (ES⁺, Short acidic): 1.04 min, m/z 305.1 [M+H]⁺ (98%)

¹H NMR (400 MHz, CDCl₃): δ 7.71 (s, 1H), 6.62-6.55 (m, 1H), 3.77 (s, 3H), 3.37 (s, 3H), 3.17 (br s, 2H), 3.13 (q, J=2.6 Hz, 2H), 2.65 (t, J=5.5 Hz, 2H), 2.53 (s, 3H), 2.36 (s, 3H)

Step 2. A solution of methyl 2-acetyl-1-methyl-5-(1-methyl-3,6-dihydro-2H-pyridin-4-yl)-6-oxo-pyridine-3-carboxylate (580 mg, 1.91 mmol) in methanol (5 mL) and acetic acid (0.4 mL) was degassed using vacuum and nitrogen (3×). Afterwards Palladium hydroxide 20% wt on carbon (136 mg, 0.19 mmol) was added and the flask was evacuated and back-filled with N₂ (3×) and finally with H₂ through the same process (3×). The reaction was stirred at rt for 6.5 hours. H₂ was removed, the mixture was filtered through celite, washed with MeOH, then evaporated to dryness. The residue was passed through an SCX washing with MeOH and eluting with NH₃ 1N in MeOH to afford methyl 2-acetyl-1-methyl-5-(1-methyl-4-piperidyl)-6-oxo-pyridine-3-carboxylate (490 mg, 1.60 mmol, 83.9% yield) as an orange oil.

UPLC-MS (ES⁺, Short acidic): 1.11 min, m/z 307.3 [M+H]⁺ (100%)

¹H NMR (400 MHz, CDCl₃): δ 7.68 (s, 1H), 3.84 (s, 3H), 3.45 (s, 3H), 3.00-2.93 (m, 2H), 2.90-2.78 (m, 1H), 2.60 (s, 3H), 2.32 (s, 3H), 2.16-2.05 (m, 2H), 1.93-1.85 (m, 2H), 1.68-1.55 (m, 2H)

Step 3. To a stirring solution of methyl 2-acetyl-1-methyl-5-(1-methyl-4-piperidyl)-6-oxo-pyridine-3-carboxylate (580 mg, 1.89 mmol) in ethanol (8 mL) was added hydrazine hydrate (230 μL, 4.73 mmol). The mixture was heated to 75° C. After 30 minutes, hydrogen chloride (316 p L, 3.79 mmol) was added and the reaction was heated to 75° C. overnight. The mixture was cooled down with an ice-water bath, filtered, washed with cold ethanol and dried in vacuum oven to afford 1,8-dimethyl-3-(1-methyl-4-piperidyl)-6H-pyrido[2,3-d]pyridazine-2,5-dione; hydrochloride (386 mg, 1.19 mmol, 62.8% yield) as an off-white solid.

UPLC-MS (ES⁺, Short acidic): 0.91 min, m/z 289.2 [M+H]⁺ (100%)

Step 4. 1,8-dimethyl-3-(1-methyl-4-piperidyl)-6H-pyrido[2,3-d]pyridazine-2,5-dione hydrochloride (341 mg, 1.05 mmol) in phosphorus oxychloride (5 mL, 53.6 mmol) was heated to 90° C. in a sealed vial for 6 hours.

The reaction was concentrated to dryness. The residue was basified with sat. aq. Na₂CO₃, extracted with DCM (×7), washed with brine and passed through a phase separator to afford 5-chloro-1,8-dimethyl-3-(1-methyl-4-piperidyl)pyrido[2,3-d]pyridazin-2-one (47 mg, 0.15 mmol, 14.6% yield) as an off-white solid.

UPLC-MS (ES⁺, Short acidic): 0.94 min, m/z 307.1/309.0 [M+H]⁺ (92%)

Step 5. A stirred solution of 5-chloro-1,8-dimethyl-3-(1-methyl-4-piperidyl)pyrido[2,3-d]pyridazin-2-one (47 mg, 0.15 mmol), (1R)-1-[3-(difluoromethyl)phenyl]ethylamine (39 mg, 0.23 mmol) and N,N-diisopropylethylamine (80 μL, 0.46 mmol) in 1-butanol (2.5 mL) was heated to 140° C. in a sealed vial for 2 days. Further (1R)-1-[3-(difluoromethyl)phenyl]ethylamine (10 mg, 0.06 mmol) was added and the reaction was heated for another 4 days. The reaction was partitioned between DCM and water. The two phases were separated, and the aqueous layer was re-extracted with DCM (×3). The combined organic extracts were washed with brine, passed through a phase separator and concentrated in vacuo. The residue was then purified by flash column chromatography (4 g, eluting with 0-20% MeOH in DCM) then by prep HPLC (intermediate method) followed by SCX chromatography washing with MeOH and eluting with NH₃ 1N in MeOH to afford 1,8-dimethyl-3-(1-methyl-4-piperidyl)-5-[[rac-(1R)-1-[3-(difluoromethyl)-phenyl]ethyl]amino]pyrido[2,3-d]pyridazin-2-one (18 mg, 0.04 mmol, 26.6% yield) as a pale yellow solid.

UPLC-MS (ES⁺, Long acidic): 2.30 min, m/z 442.3 [M+H]⁺ (93%)

¹H NMR (400 MHz, DMSO-d₆): δ 8.15 (s, 1H), 7.62-7.53 (m, 2H), 7.49-7.41 (m, 2H), 7.41-7.35 (m, 1H), 7.01 (t, J=56 Hz, 1H), 5.49 (quint, J=7.1 Hz, 1H), 3.74 (s, 3H), 2.91 (d, J=11.4 Hz, 2H), 2.86-2.76 (m, 4H), 2.22 (s, 3H), 2.04-1.90 (m, 2H), 1.85-1.67 (m, 4H), 1.57 (d, J=7.1 Hz, 3H)

### Example 14. 3-(1-acetyl-4-piperidyl)-5-[[(1R)-1-[3-(difluoromethyl)phenyl]ethyl]amino]-1,8-dimethyl-pyrido[2,3-d]pyridazin-2-one (22)

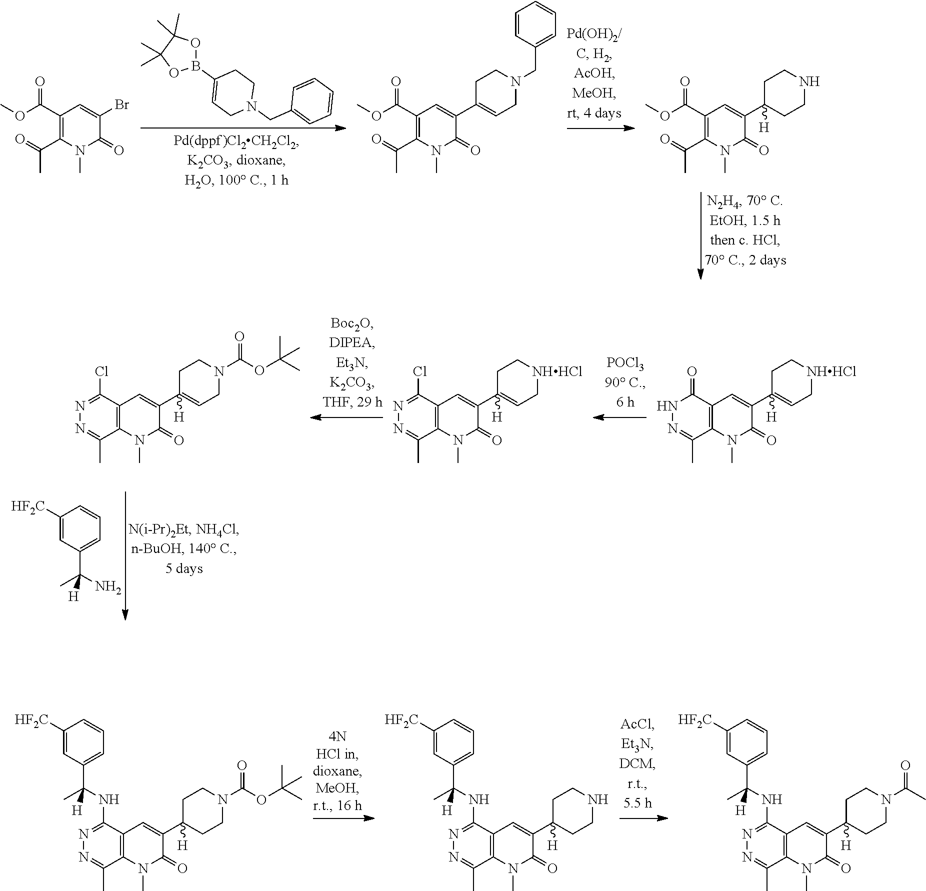

Step 1. Methyl 2-acetyl-5-bromo-1-methyl-6-oxo-pyridine-3-carboxylate (3 g, 10.4 mmol), potassium carbonate (2.88 g, 20.8 mmol) and 1-benzyl-1,2,3,6-tetrahydropyridine-4-boronic acid pinacol ester (3.43 g, 11.5 mmol) were mixed in 1,4-dioxane (8 mL) and water (1 mL) and degassed with nitrogen for 10 minutes. [1,1′-Bis(diphenylphosphino)ferrocene]Palladium(II) chloride dichloromethane complex (850 mg, 1.04 mmol) was added and the reaction was heated to 100° C. for 4.5 hours in a sealed vial.

The reaction was partitioned between DCM and water. The two phases were separated and the aqueous layer was re-extracted with DCM (×3). The combined organic extracts phase were washed with brine, passed through a phase separator and concentrated under reduced pressure. The residue was then purified by flash column chromatography (40 g, eluting in 0-5% MeOH in DCM) to afford methyl 2-acetyl-5-(1-benzyl-3,6-dihydro-2H-pyridin-4-yl)-1-methyl-6-oxo-pyridine-3-carboxylate (quantitative yield) as a brown oil.

UPLC-MS (ES⁺, Short acidic): 1.30 min, m/z 381.2 [M+H]⁺ (90%)

Step 2. A solution of methyl 2-acetyl-5-(1-benzyl-3,6-dihydro-2H-pyridin-4-yl)-1-methyl-6-oxo-pyridine-3-carboxylate (3.96 g, 10.4 mmol) and acetic acid (0.5 mL) in methanol (6 mL) and DCM (4 mL) was degassed using vacuum and nitrogen (3×). Afterwards palladium hydroxide 20% wt on carbon (741 mg, 1.04 mmol) was added and the flask was evacuated and back-filled with N₂ (3×) and finally with H₂ through the same process (3×). The reaction was stirred at rt overnight. Further palladium Hydroxide 20% wt on carbon (741 mg, 1.04 mmol) was added and the reaction was stirred for another 3 days. The mixture was filtered through celite, washed with MeOH, then evaporated to dryness. The residue was passed through a SCX cartridge (20 g) and flushed with NH₃ 1N in MeOH, to afford methyl 2-acetyl-1-methyl-6-oxo-5-(4-piperidyl)pyridine-3-carboxylate (quantitative yield) as a sticky brown solid. The compound was carried to the next step without further purification.

UPLC-MS (ES⁺, Short acidic): 1.02 min, m/z 293.1 [M+H]⁺ (84%)

Step 3. To a stirring solution of methyl 2-acetyl-1-methyl-6-oxo-5-(4-piperidyl)pyridine-3-carboxylate (3.42 g, 11.7 mmol) in ethanol (lOmL) was added hydrazine hydrate (2 mL, 41.1 mmol). The mixture was heated to 80° C. After 1.5 hours hydrogen chloride (2 mL, 24.0 mmol) was added and the reaction was heated to 80° C. for 2 days. The mixture was concentrated to afford crude 1,8-dimethyl-3-(4-piperidyl)-6H-pyrido[2,3-d]pyridazine-2,5-dione; hydrochloride as a brown wet paste which was telescoped through the next without further purification. Yield assumed quantitative.

UPLC-MS (ES⁺, Short acidic): 0.91 min, m/z 275.2 [M+H]⁺ (89%)

Step 4. 1,8-Dimethyl-3-(4-piperidyl)-6H-pyrido[2,3-d]pyridazine-2,5-dione hydrochloride (250.mg, 0.8 mmol) in phosphorus oxychloride (4 mL, 42.91 mmol) was heated to 90° C. for 6 hours. The reaction was concentrated to dryness, residual POCl₃ was removed by azeotroping with toluene (×3). This yielded 5-chloro-1,8-dimethyl-3-(4-piperidyl)pyrido[2,3-d]pyridazin-2-one; hydrochloride, yield assumed to be quantitative, as a brown paste which was taken on crude to the subsequent step without further purification.

UPLC-MS (ES⁺, Short acidic): 0.80 min, m/z 293/295 [M+H]⁺ (70%)

Step 5. To a stirring solution of 5-chloro-1,8-dimethyl-3-(4-piperidyl)pyrido[2,3-d]pyridazin-2-one (3.8 g, 13.0 mmol), di-tert-butyl dicarbonate (4.25 g, 19.5 mmol) and N,N-diisopropylethylamine (9.8 mL, 56.3 mmol) in THF (20 mL) was stirred at RT for 1 hour. LCMS indicated no product formation, therefore potassium carbonate (5.38 g, 38.9 mmol) was added. triethylamine (5 mL, 35.9 mmol) was added along with di-tert-butyl dicarbonate (4 g, 18.33 mmol). The reaction was stirred for 29 hours. The reaction was concentrated to dryness, the residue was partitioned between DCM and water and passed through celite to remove insolubles. The aqueous layer was extracted with DCM (×3). The organic phase was washed with brine, passed through a phase separator, and concentrated in vacuo. The residue was then purified by flash column chromatography twice (40 g, dry-load, eluting with 0-100% EtOAc in petroleum ether) to afford tert-butyl 4-(5-chloro-1,8-dimethyl-2-oxo-pyrido[2,3-d]pyridazin-3-yl)piperidine-1-carboxylate (1.43 g, 3.64 mmol, 28% yield—yield over 3 steps) as a brown gum.

UPLC-MS (ES⁺, Short acidic): 1.80 min, m/z 393.2/395.2 [M+H]⁺ (100%)

Step 6. A stirred solution of tert-butyl 4-(5-chloro-1,8-dimethyl-2-oxo-pyrido[2,3-d]pyridazin-3-yl)piperidine-1-carboxylate (300.mg, 0.76 mmol), (1R)-1-[3-(difluoromethyl)phenyl]ethylamine (196.08 mg, 1.15 mmol) and N,N-Diisopropylethylamine (399 μL, 2.29 mmol) in 1-Butanol (3 mL) was heated to 140° C. in a sealed vial for 5 days. The reaction was partitioned between DCM and water. The two phases were separated, and the aqueous layer was re-extracted with DCM (×3). The combined organic extracts were washed with brine, passed through a phase separator, and concentrated in vacuo. The residue was then purified by flash column chromatography twice (12 g, eluting in 0-20% MeOH in DCM) then (12 g, eluting in 0-5% MeOH in DCM) to afford tert-butyl 4-[1,8-dimethyl-2-oxo-5-[[rac-(1R)-1-[3-(difluoromethyl)phenyl]ethyl]amino]pyrido[2,3-d]pyridazin-3-yl]piperidine-1-carboxylate (124 mg, 0.24 mmol, 30.8% yield) as a brown oil.

UPLC-MS (ES⁺, Short acidic): 1.79 min, m/z 528.8 [M+H]⁺ (77%)

Step 7. To a stirring solution of tert-butyl 4-[5-[[(1R)-1-[3-(difluoromethyl)phenyl]ethyl]amino]-1,8-dimethyl-2-oxo-pyrido[2,3-d]pyridazin-3-yl]piperidine-1-carboxylate (123.mg, 0.23 mmol) in methanol (4 mL) was added hydrogen chloride 4N in dioxane (349.69 uL, 1.4 mmol) at rt. The reaction was stirred overnight. The reaction was concentrated to dryness. The residue was neutralised with NH₃ 1N MeOH, concentrated and taken on crude to the next step without further purification.

UPLC-MS (ES+, Short acidic): 1.14 min, m/z 428.3 [M+H]⁺ (67%)

Step 8. To a stirring solution of 5-[[(1R)-1-[3-(difluoromethyl)phenyl]ethyl]amino]-1,8-dimethyl-3-(4-piperidyl)pyrido[2,3-d]pyridazin-2-one (70.mg, 0.16 mmol) in dry DCM (2 mL) was added triethylamine (92. uL, 0.66 mmol) followed by acetyl chloride (16.3 μL, 0.23 mmol). The reaction was then stirred at rt for 4 hours. Further Acetyl chloride (8.0 μL, 0.11 mmol) was added and the reaction was stirred for 1.5 hours. The mixture was concentrated, then purified by flash column chromatography twice (12 g, dry-load, eluting with 0-10% MeOH in DCM) then (4 g, wet-load, eluting with 20-100% EtOAc in PET then 0-5% MeOH in DCM) to afford 3-(1-acetyl-4-piperidyl)-5-[[(1R)-1-[3-(difluoromethyl)phenyl]ethyl]amino]-1,8-dimethyl-pyrido[2,3-d]pyridazin-2-one (65 mg, 0.1384 mmol, 84.543% yield) as a brown solid.

UPLC-MS (ES+, Long acidic): 2.87 min, m/z 470.2 [M+H]⁺ (100%)

1H NMR (400 MHz, DMSO-d6): δ 8.09 (s, 1H), 7.62-7.53 (m, 2H), 7.49-7.35 (m, 3H), 7.00 (t, J=55.9 Hz, 1H), 5.48 (quint, J=7.1 Hz, 1H), 4.60 (d, J=12.8 Hz, 1H), 3.97 (d, J=13.9 Hz, 1H), 3.75 (s, 3H), 3.24-3.05 (m, 2H), 2.79 (s, 3H), 2.69-2.55 (m, 1H), 2.04 (s, 3H), 1.93-1.74 (m, 2H), 1.66-1.46 (m, 5H).

The following example was prepared in a similar manner, starting from the corresponding amines.

| Compound | Structure | Analytical data |
|---|---|---|
| 23 | 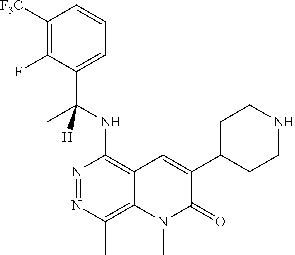 [SMILES: Cc1nnc(N[C@H](C)c2cccc(C(F)(F)F)c2F)c2cc(C3CCNCC3)c(=O)n(C)c12] | UPLC-MS (ES⁺, Long acidic): 3.03 min, m/z 464.1 [M + H]⁺ (100%) 1H NMR (400 MHz, DMSO-d6): 8.14 (s, 1H), 7.74- 7.68 (m, 1H), 7.65-7.52 (m, 2H), 7.37-7.27 (m, 1H), 5.65 (quint, J = 6.9 Hz, 1H), 3.74 (s, 3H), 3.15-3.04 (m, 2H), 3.0-2.88 (m, 1H), 2.79 (s, 3H), 2.70-2.59 (m, 2H), 1.80-1.7- (m, 2H), 1.70-1.53 (m, 5H) |

### Example 15. Biological Data of Disclosed Compounds

The capacity of compounds to inhibit SOS1 binding to KRAS-WT (wild-type) was quantified using a FRET-based protein-protein interaction assay. The assay is based on the transfer of energy between two fluorophores, a donor and an acceptor, when in close proximity. In this instance, the donor is a Europium-conjugated α-GST antibody that binds to GST-tagged KRAS-WT, and the acceptor is an XL665-conJugated α-His6 antibody that binds to His6-tagged SOS1. Binding of SOS1 to KRAS-WT results in an increased fluorescent signal at emission wavelength of 665 nm which can be detected on the EnVision plate reader. Compounds that inhibit binding will reduce the 665 nm signal emitted. Recombinant KRAS-WT protein (40 nM; Human KRAS, aal-188 recombinant protein with N-terminal GST-tag) and SOS1 protein (40 nM; Human SOS1 exchange domain, aa564-1049 with N-terminal 6His-tag) were mixed together in assay buffer (5 mM HEPES pH7.3, 150 mM NaCl, 10 mM EDTA, 5 mM MgCl₂, 0.05% BSA, 0.0025% NP-40, 1 mM DTT and 100 mM KF) and incubated at room temperature with a dose response of compound in a 384-well low volume white plate and a final volume of 5 ul. After a 60 minute incubation, 5 ul of 4 nM anti-GST-Eu(K) (Cisbio, France) combined with 20 nM anti-6His-XL665 (Cisbio, France), diluted in assay buffer, was added to the plate. Following a further 4 h incubation at room temperature, time-resolved fluorescence was measured on the EnVision plate reader. DMSO (0.05%) and 10 μM reference compound were used to generate the Max and Min assay signals, respectively. Data was analyzed using a four-parameter logistic model to calculate IC₅₀ values, with at least two independent replicates performed for each compound.

Data was analyzed using a four-parameter logistic model to calculate IC₅₀ values, with at least two independent replicates were performed for each compound.

TABLE 3
SOS1/KRAS WT HTRF binding assay data

| | | | SOS1/KRAS WT |
|---|---|---|---|
| | | | HTRF Binding |
| | Name | Structure | assay pIC50 |
| 1 | 6-cyclopentyl-1-methyl-4-[1-[3- (trifluoromethyl)phenyl]ethylamino] pyrido[3,4-d]pyridazin-7-one | 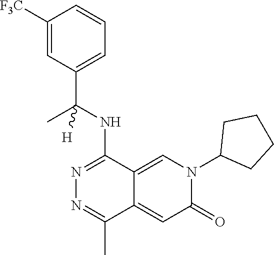 [SMILES: Cc1nnc(NC(C)c2cccc(C(F)(F)F)c2)c2cn(C3CCCC3)c(=O)cc12] | 6.72 |
| 2 | 6-cyclopentyl-1-methyl-4-[[(1R)-1- [3- (trifluoromethyl)phenyl]ethyl]amino] pyrido[3,4-d]pyridazin-7-one | 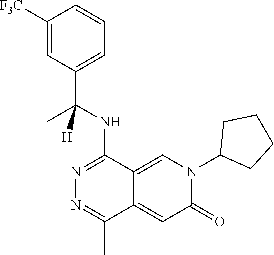 [SMILES: Cc1nnc(N[C@H](C)c2cccc(C(F)(F)F)c2)c2cn(C3CCCC3)c(=O)cc12] | 7.11 |
| 3 | 6-ethyl-1-methyl-4-[[(1R)-1-[3- (trifluoromethyl)phenyl]ethyl]amino] pyrido[3,4-d]pyridazin-7-one | 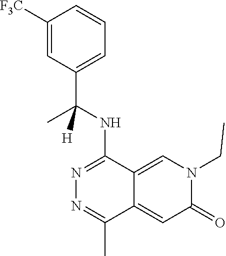 [SMILES: CCn1cc2c(N[C@H](C)c3cccc(C(F)(F)F)c3)nnc(C)c2cc1=O] | 6.46 |
| 4 | 6-(cyclopropylmethyl)-1-methyl-4- [[(1R)-1-[3- (trifluoromethyl)phenyl]ethyl]amino] pyrido[3,4-d]pyridazin-7-one | 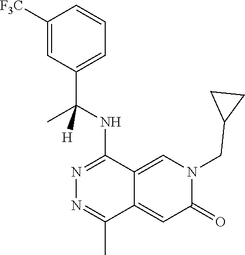 [SMILES: Cc1nnc(N[C@H](C)c2cccc(C(F)(F)F)c2)c2cn(CC3CC3)c(=O)cc12] | 6.52 |
| 5 | 1-methyl-6-(oxetan-3-ylmethyl)-4-[[(1R)- 1-[3- (trifluoromethyl)phenyl]ethyl]amino] pyrido[3,4-d]pyridazin-7-one | 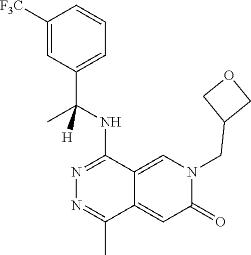 [SMILES: Cc1nnc(N[C@H](C)c2cccc(C(F)(F)F)c2)c2cn(CC3COC3)c(=O)cc12] | 6.52 |
| 6 | 1-methyl-6-[(3-methyloxetan-3- yl)methyl]-4-[[(1R)-1-[3- (trifluoromethyl)phenyl]ethyl]amino] pyrido[3,4-d]pyridazin-7-one | 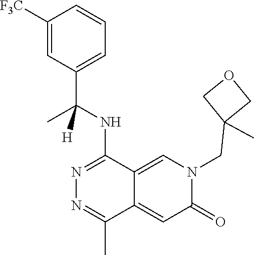 [SMILES: Cc1nnc(N[C@H](C)c2cccc(C(F)(F)F)c2)c2cn(CC3(C)COC3)c(=O)cc12] | 6.34 |
| 7 | 6-isopropyl-1-methyl-4-[[(1R)-1-[3- (trifluoromethyl)phenyl]ethyl]amino] pyrido[3,4-d]pyridazin-7-one | 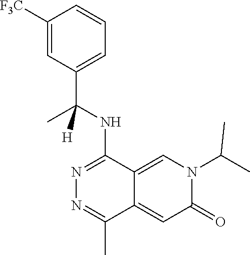 [SMILES: Cc1nnc(N[C@H](C)c2cccc(C(F)(F)F)c2)c2cn(C(C)C)c(=O)cc12] | 7.02 |
| 8 | 1-methyl-4-[[rac-(1R)-1-[3- (trifluoromethyl)phenyl]ethyl]amino]-6- tetrahydrofuran-3-yl-pyrido[3,4- d]pyridazin-7-one | 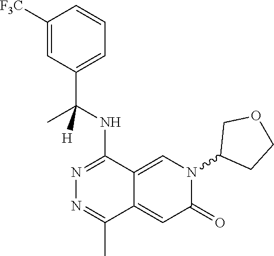 [SMILES: Cc1nnc(N[C@H](C)c2cccc(C(F)(F)F)c2)c2cn(C3CCOC3)c(=O)cc12] | 6.63 |
| 9 | 8-methyl-3-[(3S)-tetrahydrofuran-3- yl]oxy-N-[1-[3- (trifluoromethyl)phenyl]ethyl]pyrido[2,3- d]pyridazin-5-amine | 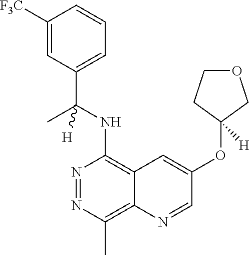 [SMILES: Cc1nnc(NC(C)c2cccc(C(F)(F)F)c2)c2cc(O[C@H]3CCOC3)cnc12] | 6.60 |
| 10 | 8-methyl-3-[(3S)-tetrahydrofuran-3- yl]oxy-N-[(1R)-1-[3- (trifluoromethyl)phenyl]ethyl]pyrido[2,3- d]pyridazin-5-amine | 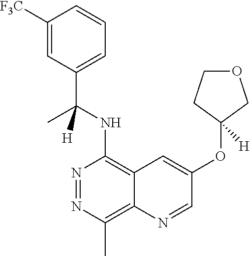 [SMILES: Cc1nnc(N[C@H](C)c2cccc(C(F)(F)F)c2)c2cc(O[C@H]3CCOC3)cnc12] | 6.76 |
| 11 | 3-(cyclopenten-1-yl)-5-[[(1R)-1-[3- (difluoromethyl)phenyl]ethyl]amino]-1,8- dimethyl-pyrido[2,3-d]pyridazin-2-one | 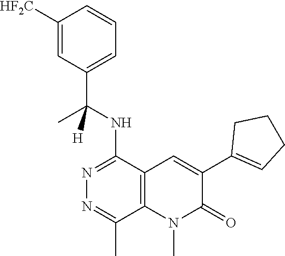 | 6.75 |
| 12 | 3-(cyclopenten-1-yl)-1,8-dimethyl-5- [[(1R)-1-[3- (trifluoromethyl)phenyl]ethyl]amino] pyrido[2,3-d]pyridazin-2-one | 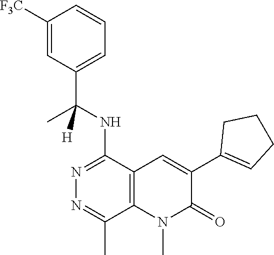 [SMILES: Cc1nnc(N[C@H](C)c2cccc(C(F)(F)F)c2)c2cc(C3=CCCC3)c(=O)n(C)c12] | 6.96 |
| 13 | 6-cyclopropyl-1-methyl-4-[[(1R)-1-[3- (trifluoromethyl)phenyl]ethyl]amino] pyrido[3,4-d]pyridazin-7-one | 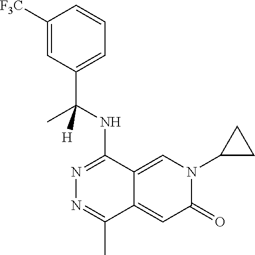 [SMILES: Cc1nnc(N[C@H](C)c2cccc(C(F)(F)F)c2)c2cn(C3CC3)c(=O)cc12] | 7.20 |
| 14 | 1-methyl-6-(1-methylcyclopropyl)-4- [[rac-(1R)-1-[3- (trifluoromethyl)phenyl]ethyl]amino] pyrido[3,4-d]pyridazin-7-one | 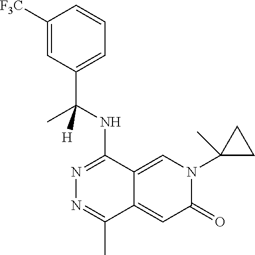 [SMILES: Cc1nnc(N[C@H](C)c2cccc(C(F)(F)F)c2)c2cn(C3(C)CC3)c(=O)cc12] | 7.23 |
| 15 | 5-methyl-2-[(3S)-tetrahydrofuran-3- yl]oxy-N-[1-[3- (trifluoromethyl)phenyl]ethyl]pyrido[2,3- d]pyridazin-8-amine | 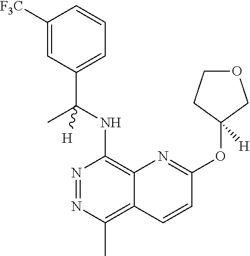 [SMILES: Cc1nnc(NC(C)c2cccc(C(F)(F)F)c2)c2nc(O[C@H]3CCOC3)ccc12] | 6.17 |
| 16 | 1,8-dimethyl-3-(1,2,3,6- tetrahydropyridin-5-yl)-5-[[(1R)-1-[3- (trifluoromethyl)phenyl]ethyl]amino] pyrido[2,3-d]pyridazin-2-one | 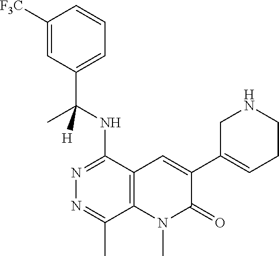 [SMILES: Cc1nnc(N[C@H](C)c2cccc(C(F)(F)F)c2)c2cc(C3=CCCNC3)c(=O)n(C)c12] | 7.30 |
| 17 | 1,8-dimethyl-3-(3-piperidyl)-5-[[(1R)-1- [3- (trifluoromethyl)phenyl]ethyl]amino] pyrido[2,3-d]pyridazin-2-one | 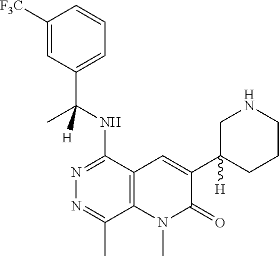 [SMILES: Cc1nnc(N[C@H](C)c2cccc(C(F)(F)F)c2)c2cc(C3CCCNC3)c(=O)n(C)c12] | 7.47 |
| 18 | 3-(1-acetyl-3-piperidyl)-1,8-dimethyl-5- [[(1R)-1-[3- (trifluoromethyl)phenyl]ethyl]amino] pyrido[2,3-d]pyridazin-2-one | 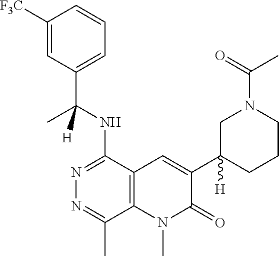 [SMILES: CC(=O)N1CCCC(c2cc3c(N[C@H](C)c4cccc(C(F)(F)F)c4)nnc(C)c3n(C)c2=O)C1] | 7.36 |
| 19 | 3-(1-acetyl-3,6-dihydro-2H-pyridin-4-yl)- 1,8-dimethyl-5-[[rac-(1R)-1-[3- (trifluoromethyl)phenyl]ethyl]amino] pyrido[2,3-d]pyridazin-2-one | 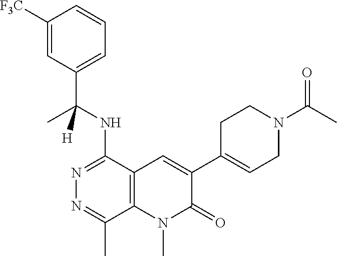 [SMILES: CC(=O)N1CC=C(c2cc3c(N[C@H](C)c4cccc(C(F)(F)F)c4)nnc(C)c3n(C)c2=O)CC1] | 7.44 |
| 20 | 1,8-dimethyl-5-[[rac-(1R)-1-[3- (difluoromethyl)phenyl]ethyl]amino]-3- tetrahydropyran-4-yl-pyrido[2,3- d]pyridazin-2-one | 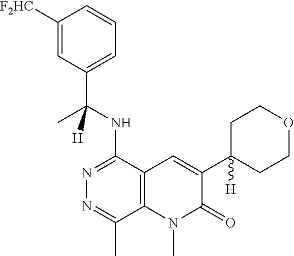 [SMILES: Cc1nnc(N[C@H](C)c2cccc(C(F)F)c2)c2cc(C3CCOCC3)c(=O)n(C)c12] | 7.03 |
| 21 | 1,8-dimethyl-3-(1-methyl-4-piperidyl)-5- [[rac-(1R)-1-[3- (difluoromethyl)phenyl]ethyl]amino] pyrido[2,3-d]pyridazin-2-one | 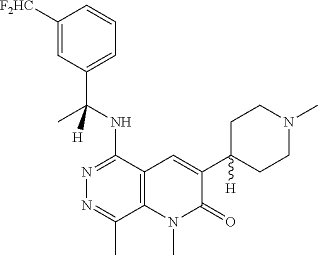 [SMILES: Cc1nnc(N[C@H](C)c2cccc(C(F)F)c2)c2cc(C3CCN(C)CC3)c(=O)n(C)c12] | 7.27 |
| 22 | 3-(1-acetyl-4-piperidyl)-5-[[(1R)-1-[3- (difluoromethyl)phenyl]ethyl]amino]-1,8- dimethyl-pyrido[2,3-d]pyridazin-2-one | 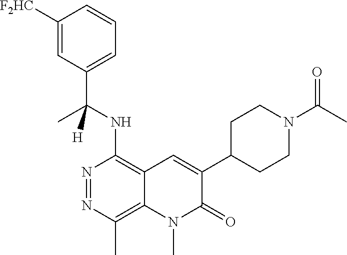 [SMILES: CC(=O)N1CCC(c2cc3c(N[C@H](C)c4cccc(C(F)F)c4)nnc(C)c3n(C)c2=O)CC1] | 7.45 |
| 23 | 5-[[(1R)-1-[2-fluoro-3- (trifluoromethyl)phenyl]ethyl]amino]-1,8- dimethyl-3-(4-piperidyl)pyrido[2,3- d]pyridazin-2-one | 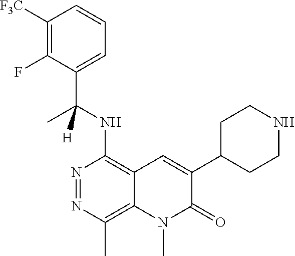 [SMILES: Cc1nnc(N[C@H](C)c2cccc(C(F)(F)F)c2F)c2cc(C3CCNCC3)c(=O)n(C)c12] | 7.24 |

What is claimed is:

1. A compound of Formula (Ia): 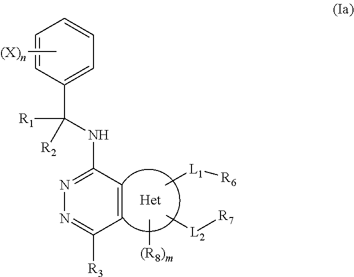
or a pharmaceutically acceptable salt thereof,
wherein:
R₁ and R₂ are independently hydrogen, alkyl, or R₁ and R₂ together with the atom to which they are attached form a cycloalkyl or heterocyclyl, wherein at least one of R₁ and R₂ is not hydrogen;
R₃ is hydrogen, alkyl, —(C═O)—OR_{A}, —(C═O)—N(R_{A})₂, cycloalkyl, heterocyclyl, aryl or heteroaryl; wherein R_{A} is hydrogen or alkyl; 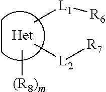
is a heteroaryl;
R₆ is H, alkyl, cycloalkyl, cycloalkenyl, heterocyclyl, aryl or heteroaryl;
R₇ is H, halogen, alkyl, cycloalkyl, heterocyclyl, aryl, or heteroaryl;
R₈ is H, halogen, alkyl, alkoxy, alkylene-O-alkyl, cycloalkyl, or heterocyclyl;
L₁ and L₂ are each independently absent or a linking group; and
X is independently selected from the group consisting of C₁₋₅alkyl, F, CF₃, CHF₂, CH₂F, and —NH₂;
m is 1 or 2; and
n is an integer from 1-5,
provided that:
(a) 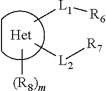
is not 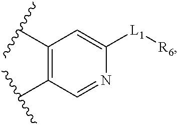
and
(b) the compound of Formula (Ia) is not:

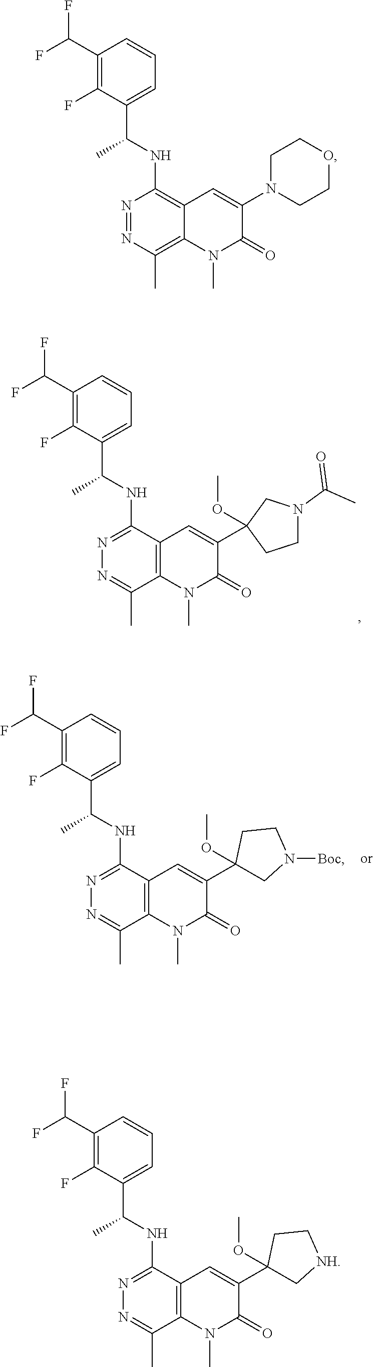 [SMILES: Cc1nnc(N[C@H](C)c2cccc(C(F)F)c2F)c2cc(N3CCOCC3)c(=O)n(C)c12 ; COC1(c2cc3c(N[C@H](C)c4cccc(C(F)F)c4F)nnc(C)c3n(C)c2=O)CCN(C(C)=O)C1 ; COC1(c2cc3c(N[C@H](C)c4cccc(C(F)F)c4F)nnc(C)c3n(C)c2=O)CCN(C(=O)OC(C)(C)C)C1 ; COC1(c2cc3c(N[C@H](C)c4cccc(C(F)F)c4F)nnc(C)c3n(C)c2=O)CCNC1]

2. The compound of claim 1, wherein 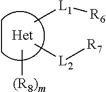
is selected from the group consisting of: 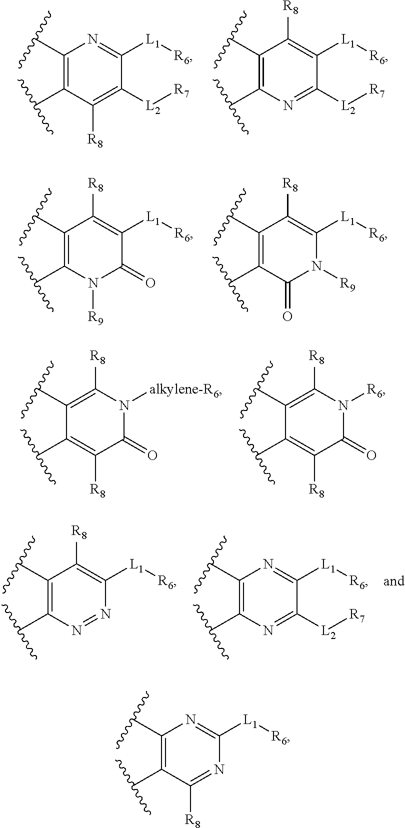
wherein R₉ is H, alkyl, cycloalkyl, heterocyclyl, alkylene-cycloalkyl, or alkylene-heterocyclyl.

3. The compound of claim 1 or 2, wherein the linking group is —O—, alkylene, alkylene-O—, alkylene-N(R_{B})—, —O-alkylene, —N(R_{B})-alkylene, —O—, or —N(R_{B})—, wherein R_{B} is hydrogen, alkyl, or alkylenecycloalkyl.

4. The compound of claim 1 or 2, wherein L₁ and L₂ are each independently absent or a linking group selected from the group consisting of alkylene, —O-alkylene, —N(R_{B})-alkylene, —O—, and —N(R_{B})—, wherein R_{B} is hydrogen, alkyl, or alkylenecycloalkyl.

5. The compound of any one of claims 1-4, wherein L₁ is absent or a linking group selected from alkylene, —O-alkylene, —N(R_{B})-alkylene, —O—, and —N(R_{B})— and L₂ is absent, —O—, or —N(R_{B})—, wherein R_{B} is hydrogen, alkyl, or alkylenecycloalkyl.

6. The compound of any one of claims 1-4, wherein L₁ is a linking group selected from alkylene, —O-alkylene, —N(R_{B})-alkylene, —O—, and —N(R_{B})—, wherein R_{B} is hydrogen, alkyl, or alkylenecycloalkyl and L₂ is absent or —O—.

7. The compound of any one of claims 1-4, wherein L₁ is alkylene or —O— and L₂ is absent or —O—.

8. The compound of any one of claims 1-4, wherein L₁ and L₂ are —O—.

9. The compound of any one of claims 1-4, wherein L₁ is -alkylene- and L₂ is absent.

10. The compound of any one of claims 1-9, wherein R₆ is alkyl, cycloalkyl, cycloalkenyl, or heterocyclyl.

11. The compound of any one of claims 1-9, wherein R₆ is C₁₋₅alkyl, C₃₋₆cycloalkyl, or 6-membered heterocyclyl having a heteroatom selected from N, O, and S.

12. The compound of any one of claims 1-9, wherein R₆ is heterocyclyl.

13. The compound of any one of claims 1-9, wherein R₆ is a 5- or 6-membered heterocyclyl having 1 or 2 heteroatoms selected from N, O, and S.

14. The compound of any one of claims 1-9, wherein R₆ is a morpholinyl, piperazinyl, piperidinyl, pyrrolidinyl, azetidinyl, tetrahydropyranyl, or tetrahydrofuranyl.

15. The compound of any one of claims 1-9, wherein R₆ is a piperazinyl, piperidinyl, azetidinyl, tetrahydropyranyl, or tetrahydrofuranyl.

16. The compound of any one of claims 1-9, wherein R₆ is 3-tetrahydrofuranyl, 3-oxetanyl, 3-piperidinyl, 4-piperidinyl, or 4-tetrahydropyranyl.

17. The compound of any one of claims 1-9, wherein R₆ is alkyl or cycloalkyl.

18. The compound of any one of claims 1-9, wherein R₆ is cyclopentyl or cyclopentenyl.

19. The compound of claim 1-9, wherein R₆ is methyl, ethyl, or isopropyl.

20. The compound of any one of claims 1-9, wherein R₆ is:

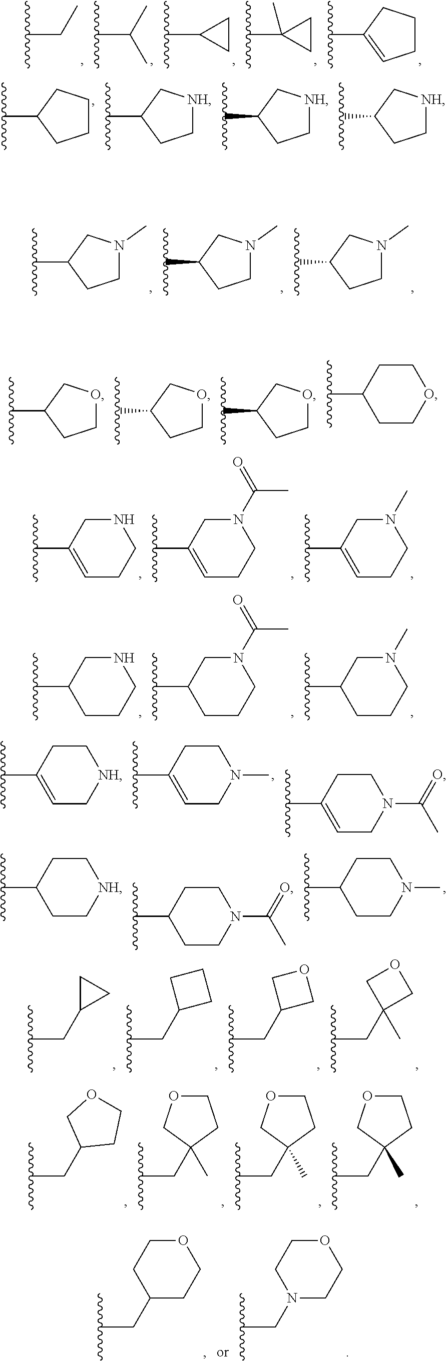

21. The compound of any one of claims 1-20, wherein R₇ is H, halogen, C₁₋₅alkyl, C₃₋₆cycloalkyl, C₄₋₆heterocyclyl, or 5-6-membered heteroaryl.

22. The compound of any one of claims 1-20, wherein R₇ is cyclopentyl or 3-tetrahydrofuranyl.

23. The compound of any one of claims 1-20, wherein R₇ is cyclopentyl.

24. The compound of any one of claims 1-20, wherein R₇ is morpholinyl, piperazinyl, piperidinyl, pyrrolidinyl, or azetidinyl.

25. The compound of any one of claims 1-20, wherein R₇ is pyrazolyl, pyridinyl, pyrimidinyl, imidazolyl, oxazolyl, or thiazolyl.

26. The compound of any one of claims 1-9, wherein R₆ is alkyl, cycloalkyl, cycloalkenyl, heterocyclyl, aryl or heteroaryl and R₇ is H, halogen, or alkyl.

27. The compound of any one of claims 1-9, wherein R₆ is alkyl, cycloalkyl, cycloalkenyl, or heterocyclyl, and R₇ is H, halogen, or alkyl.

28. The compound of any one of claims 1-9, wherein R₇ is alkyl, cycloalkyl, heterocyclyl, aryl or heteroaryl and R₆ is H or alkyl.

29. The compound of any one of claims 1-28, wherein R₈ is H, halogen, C₁₋₅alkyl, C₁₋₅alkoxy, or —CH₂—O—C₁₋₅alkyl.

30. The compound of any one of claims 1-29, wherein R₉ is H or CH₃.

31. The compound of any one of claims 1-30, wherein each X is independently —CF₂CH₃, —CF₂CH₂OH, —CF₂C(CH₃)₂OH, —CHF₂, —CF₃, F, or —NH₂.

32. The compound of any one of claims 1-31, wherein n is 1.

33. The compound of any one of claims 1-31, wherein n is 2.

34. The compound of any one of claims 1-33, wherein 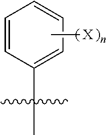
is: 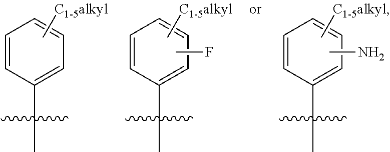
wherein the C₁₋₅alkyl is a C₁₋₅haloalkyl.

35. The compound of claim 34, wherein the C₁₋₅alkyl is a C₁₋₅fluoroalkyl.

36. The compound of claim 33 or 34, wherein the C₁₋₅alkyl is selected from the group consisting of —CF₂CF₃, —CF₂CH₃, —CF₂CH₂OH, —CF₂C(CH₃)₂OH, —CHF₂, —CF₃, and —CH₂F.

37. The compound of any one of claims 1-33, wherein 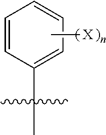
is:

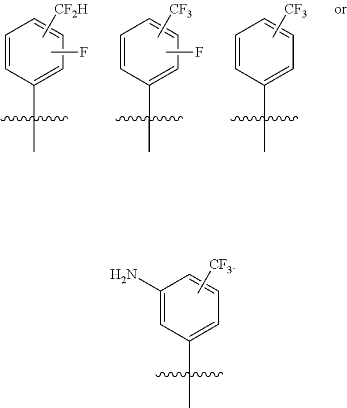

38. The compound of any one of claims 1-33, wherein 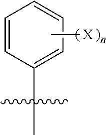
is:

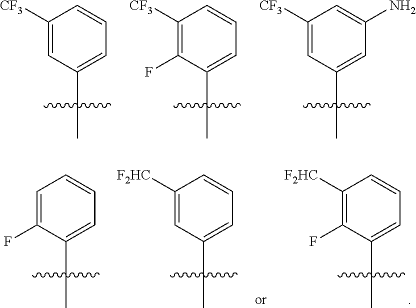

39. The compound of any one of claims 1-38, wherein 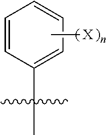
is

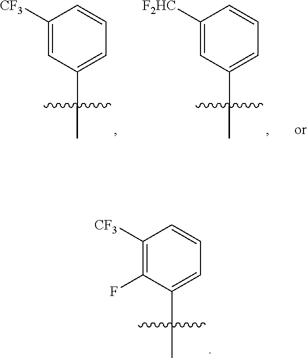 [SMILES: *c1cccc(C(F)(F)F)c1 ; *c1cccc(C(F)F)c1 ; *c1cccc(C(F)(F)F)c1F]

40. The compound of any one of claims 1-39, wherein R₃ is C₁₋₅alkyl.

41. The compound of any one of claims 1-39, wherein R₃ is methyl, ethyl, isopropyl, n-propyl, —CH₂OH, —CH₂OCH₃, —CH₂N(CH₃)₂, —CH(OH)(CH₃)₂ or —CH₂(OH)CH₃.

42. The compound of any one of claims 1-41, wherein R₃ is methyl.

43. The compound of any one of claims 1-42, wherein R₁ is methyl and R₂ is H.

44. The compound of any one of claims 1-42, wherein R₁ and R₂ together with the atom to which they are attached form a C₃₋₆cycloalkyl.

45. The compound of any one of claims 1-42, wherein R₁ and R₂ together with the atom to which they are attached form a cyclopropyl.

46. The compound of any one of claims 1-39 and 42-45, having the structure of Formula (Ic-2) or Formula (Id): 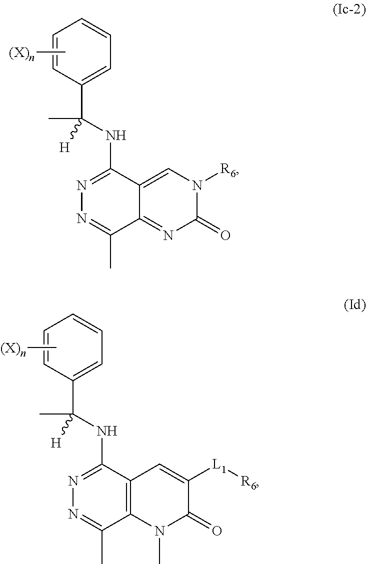
or a pharmaceutically acceptable salt thereof.

47. The compound of any one of claims 1-39 and 42-45, wherein the compound is selected from the group consisting of: 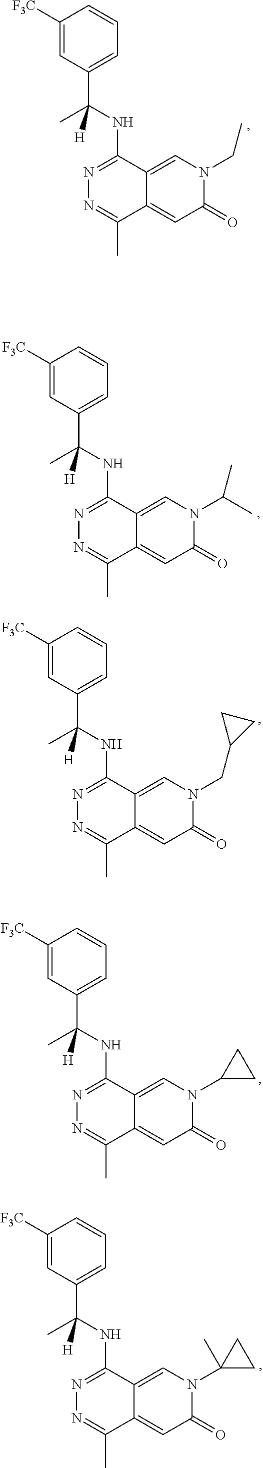 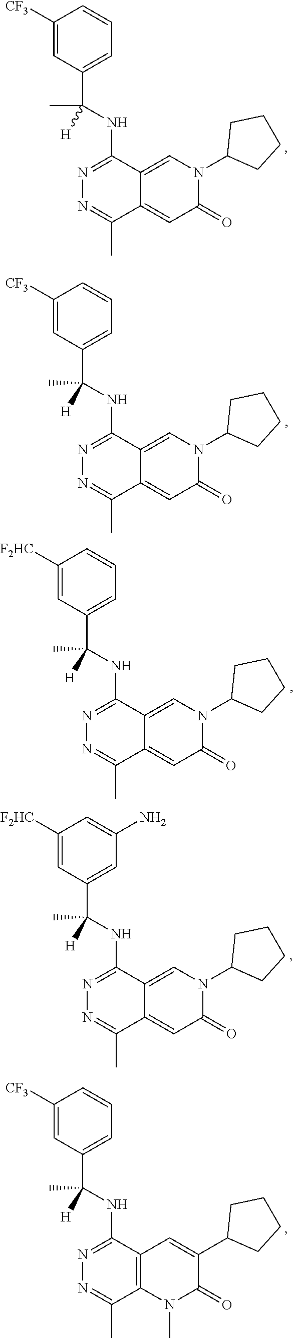 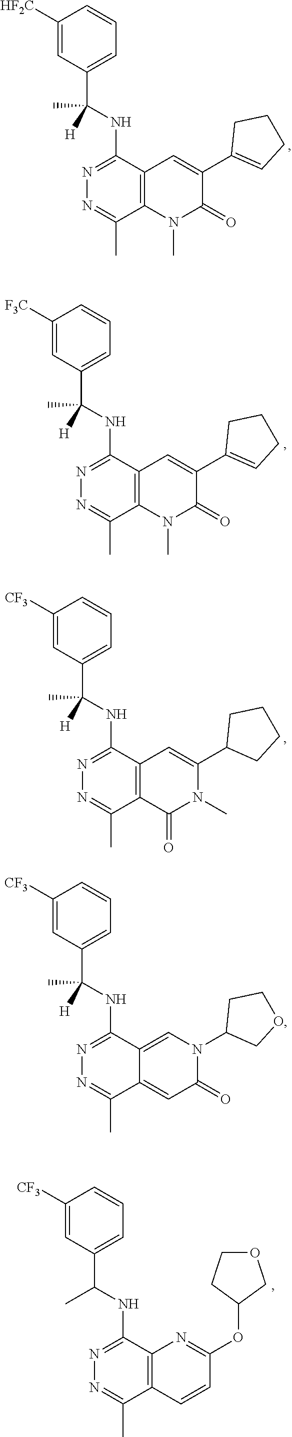 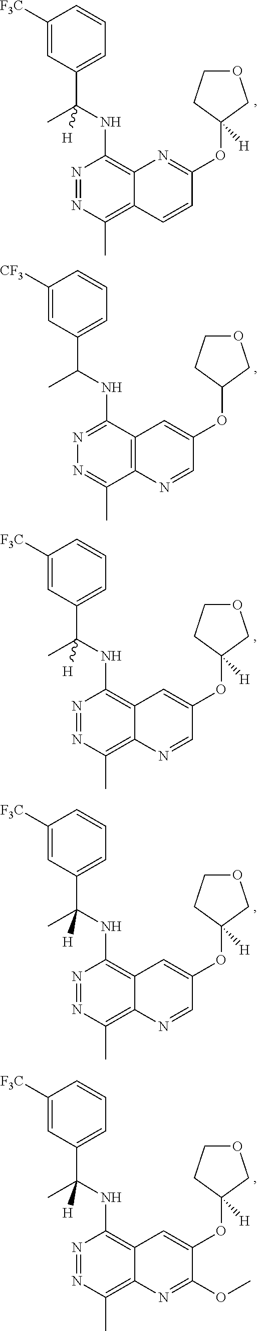 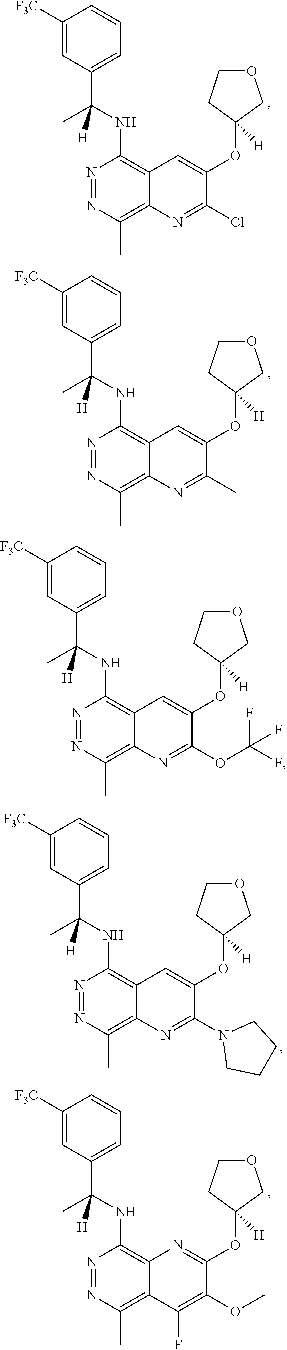 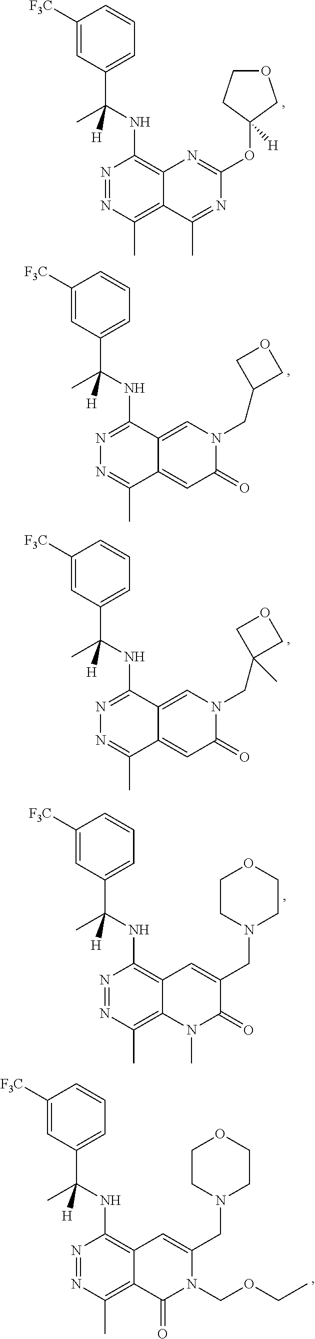 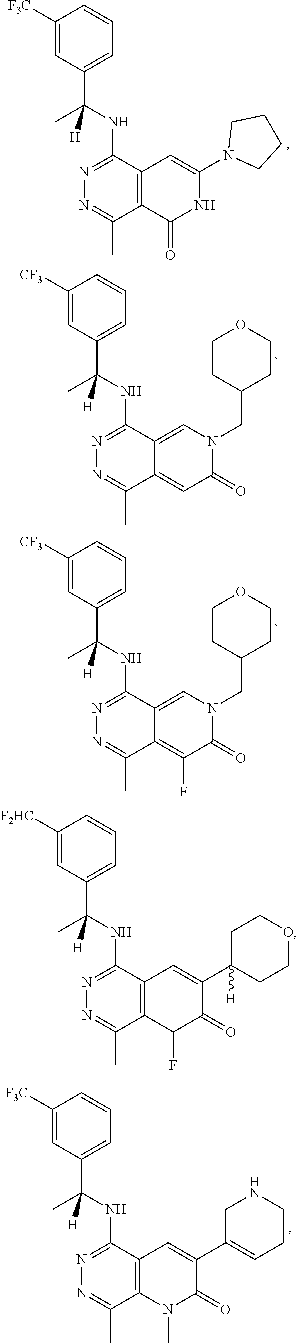 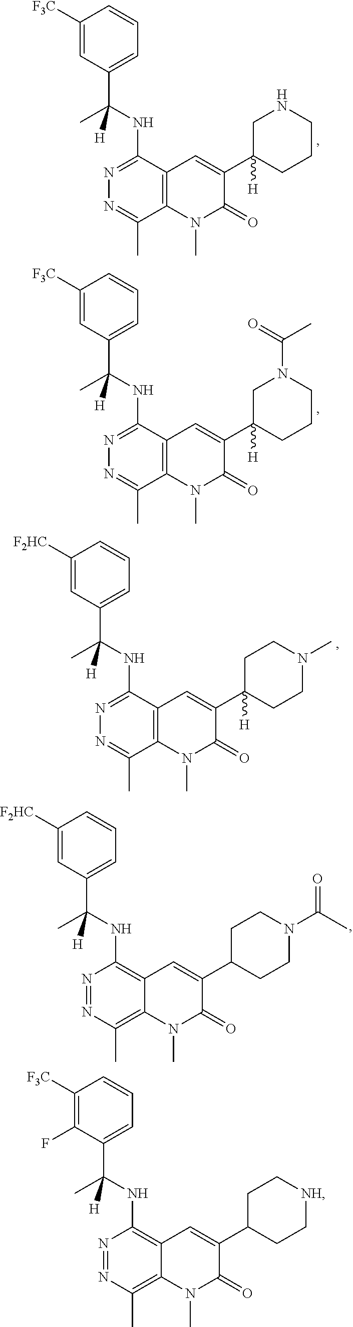
and a pharmaceutically acceptable salt thereof.

48. A composition comprising a compound or pharmaceutically acceptable salt of any one of claims 1-47 and a pharmaceutically acceptable excipient.

49. A method of treating a condition associated with or modulated by SOS1, comprising administering to a subject in need thereof a therapeutically effective amount of a compound of any one of claims 1-47 or a composition of claim 48.

50. A method of treating cancer, comprising administering to a subject in need thereof a therapeutically effective amount of a compound of any one of claims 1-47 or a composition of claim 48.

51. The compound of any one of claims 1-47 or the composition of claim 48 for use in the treatment of a condition associated with or modulated by SOS1.

52. The compound of any one of claims 1-47 or the composition of claim 48 for use in the treatment of cancer.

53. Use of the compound of any one of claims 1-47 or the composition of claim 48 in the manufacture of a medicament for the treatment of a condition associated with or modulated by SOS1.

54. Use of the compound of any one of claims 1-47 or the composition of claim 48 in the manufacture of a medicament for the treatment of cancer.

55. The compound of any one of claims 1-47 or the composition of claim 48 for use in therapy.

56. The compound of any one of claims 1-47 or the composition of claim 48 for use in a method of treating a condition associated with or modulated by SOS1.

57. The compound of any one of claims 1-47 or the composition of claim 48 for use in a method of treating cancer.
